SEQUENCE LISTING (32 sequences)

SEQ ID NO: 1 - protein, 8 aa, synthetic construct
  REGION 1..8: note = Synthetic Construct
GFTFSNYA

SEQ ID NO: 2 - protein, 8 aa, synthetic construct
  REGION 1..8: note = Synthetic Construct
ISGSGDYT

SEQ ID NO: 3 - protein, 11 aa, synthetic construct
  REGION 1..11: note = Synthetic Construct
ARSPWGYYLDS

SEQ ID NO: 4 - protein, 6 aa, synthetic construct
  REGION 1..6: note = Synthetic Construct
QGISSR

SEQ ID NO: 5 - skipped

SEQ ID NO: 6 - protein, 9 aa, synthetic construct
  REGION 1..9: note = Synthetic Construct
QQYNSYPYT

SEQ ID NO: 7 - protein, 118 aa, synthetic construct
  REGION 1..118: note = Synthetic Construct
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSS

SEQ ID NO: 8 - protein, 107 aa, synthetic construct
  REGION 1..107: note = Synthetic Construct
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIK

SEQ ID NO: 9 - protein, 25 aa, synthetic construct
  REGION 1..25: note = Synthetic Construct
EVQLLESGGGLVQPGGSLRLSCAAS

SEQ ID NO: 10 - protein, 17 aa, synthetic construct
  REGION 1..17: note = Synthetic Construct
MSWVRQAPGKGLEWVSS

SEQ ID NO: 11 - protein, 38 aa, synthetic construct
  REGION 1..38: note = Synthetic Construct
YYTDSVKGRFTISRDNSKNTLYLQMNSLRAEDTAVYYC

SEQ ID NO: 12 - protein, 11 aa, synthetic construct
  REGION 1..11: note = Synthetic Construct
WGQGTLVTVSS

SEQ ID NO: 13 - protein, 26 aa, synthetic construct
  REGION 1..26: note = Synthetic Construct
DIQMTQSPPSLSASAGDRVTITCRAS

SEQ ID NO: 14 - protein, 17 aa, synthetic construct
  REGION 1..17: note = Synthetic Construct
LAWYQQKPEKAPKSLIY

SEQ ID NO: 15 - protein, 36 aa, synthetic construct
  REGION 1..36: note = Synthetic Construct
SLQSGVPSRFSGSGSGTDFTLTISSLQPEDFATYYC

SEQ ID NO: 16 - protein, 10 aa, synthetic construct
  REGION 1..10: note = Synthetic Construct
FGQGTKLEIK

SEQ ID NO: 17 - protein, 5 aa, synthetic construct
  REGION 1..5: note = Synthetic Construct
NSGMH

SEQ ID NO: 18 - protein, 17 aa, synthetic construct
  REGION 1..17: note = Synthetic Construct
VIWYDGSKRYYADSVKG

SEQ ID NO: 19 - protein, 4 aa, synthetic construct
  REGION 1..4: note = Synthetic Construct
NDDY

SEQ ID NO: 20 - protein, 11 aa, synthetic construct
  REGION 1..11: note = Synthetic Construct
RASQSVSSYLA

SEQ ID NO: 21 - protein, 7 aa, synthetic construct
  REGION 1..7: note = Synthetic Construct
DASNRAT

SEQ ID NO: 22 - protein, 9 aa, synthetic construct
  REGION 1..9: note = Synthetic Construct
QQSSNWPRT

SEQ ID NO: 23 - protein, 30 aa, synthetic construct
  REGION 1..30: note = Synthetic Construct
QVQLVESGGGVVQPGRSLRLDCKASGITFS

SEQ ID NO: 24 - protein, 14 aa, synthetic construct
  REGION 1..14: note = Synthetic Construct
WVRQAPGKGLEWVA

SEQ ID NO: 25 - protein, 32 aa, synthetic construct
  REGION 1..32: note = Synthetic Construct
RFTISRDNSKNTLFLQMNSLRAEDTAVYYCAT

SEQ ID NO: 26 - protein, 11 aa, synthetic construct
  REGION 1..11: note = Synthetic Construct
WGQGTLVTVSS

SEQ ID NO: 27 - protein, 23 aa, synthetic construct
  REGION 1..23: note = Synthetic Construct
EIVLTQSPATLSLSPGERATLSC

SEQ ID NO: 28 - protein, 15 aa, synthetic construct
  REGION 1..15: note = Synthetic Construct
WYQQKPGQAPRLLIY

SEQ ID NO: 29 - protein, 32 aa, synthetic construct
  REGION 1..32: note = Synthetic Construct
GIPARFSGSGSGTDFTLTISSLEPEDFAVYYC

SEQ ID NO: 30 - protein, 10 aa, synthetic construct
  REGION 1..10: note = Synthetic Construct
FGQGTKVEIK

SEQ ID NO: 31 - protein, 113 aa, synthetic construct [sequence omitted]

SEQ ID NO: 32 - protein, 107 aa, synthetic construct [sequence omitted]

(19) United States
(12) Patent Application Publication
(10) Pub. No.: US 2025/0066507 A1
(43) Pub. Date: Feb. 27, 2025

(54) METHODS OF TREATING CANCER WITH A COMBINATION OF AN ANTI-PD-1 ANTIBODY AND AN ANTI-TISSUE FACTOR ANTIBODY-DRUG CONJUGATE

(71) Applicant: Genmab A/S, Valby (DK)

(72) Inventors: Reshma Abdulla RANGWALA, Philadelphia, PA (US); Esther C.W. BREIJ, Utrecht (NL); Sandra VERPLOEGEN, Nieuwegein (NL); OyewaleV O. ABIDOYE, Bellevue, WA (US); Leonardo Viana NICACIO, Bothell, WA (US); Anthony CAO, Bothell, WA (US); Shyra GARDAI, Bothell, WA (US)

(21) Appl. No.: 18/798,557

(22) Filed: Aug. 8, 2024

Related U.S. Application Data
(63) Continuation of application No. 17/053,346, filed on Nov. 5, 2020, now abandoned.
(60) Provisional application No. 62/668,104, filed on May 7, 2018.

(51) Int. Cl. C07K 16/36; A61K 45/06; A61K 47/68; A61P 35/00; C07K 16/28

(52) U.S. Cl. CPC C07K 16/36; A61K 45/06; A61K 47/68031; A61K 47/6843; A61P 35/00; C07K 16/2818

(57) ABSTRACT

The invention provides an anti-PD-1 antibody in combination with an antibody-drug conjugate that binds to tissue factor (TF) (e.g., tisotumab vedotin) and their use in methods of treating cancer, such as breast cancer and cervical cancer. The invention also provides compositions and kits comprising the anti-PD-1 antibody and the antibody-drug conjugate that binds to TF (e.g., tisotumab vedotin) for use in treating cancer, such as breast cancer and cervical cancer.

### CROSS-REFERENCE TO RELATED APPLICATIONS

This application is a continuation of U.S. application Ser. No. 17/053,346, claiming the International Filing Date May 7, 2019, which is a national stage application under 35 U.S.C. § 371 of International Application No. PCT/US2019/031168, filed internationally on May 7, 2019, which claims priority to U.S. Provisional Application 62/668,104 filed May 7, 2018, the contents of which are incorporated herein by reference in their entirety.

### REFERENCE TO AN ELECTRONIC SEQUENCE LISTING

The contents of the electronic sequence listing (761682000801seqlist.xml; Size: 40,404 bytes; and Date of Creation: Jul. 30, 2024) is herein incorporated by reference in its entirety.

### TECHNICAL FIELD

The present invention relates to methods of treating cancer, such as breast cancer and cervical cancer, with a combination of an anti-PD-1 antibody and an anti-Tissue Factor (anti-TF) antibody-drug conjugate.

### BACKGROUND

Tissue factor (TF), also called thromboplastin, factor III or CD142 is a protein present in subendothelial tissue, platelets, and leukocytes necessary for the initiation of thrombin formation from the zymogen prothrombin. Thrombin formation ultimately leads to the coagulation of blood. TF enables cells to initiate the blood coagulation cascades, and it functions as the high-affinity receptor for the coagulation factor VII (FVII), a serine protease. The resulting complex provides a catalytic event that is responsible for initiation of the coagulation protease cascades by specific limited proteolysis. Unlike the other cofactors of these protease cascades, which circulate as nonfunctional precursors, TF is a potent initiator that is fully functional when expressed on cell surfaces.

TF is the cell surface receptor for the serine protease factor VIIa (FVIIa). Binding of FVIIa to TF starts signaling processes inside the cell, said signaling function playing a role in angiogenesis. Whereas angiogenesis is a normal process in growth and development, as well as in wound healing, it is also a fundamental step in the transition of tumors from a dormant state to a malignant state. When cancer cells gain the ability to produce proteins that participate in angiogenesis (i.e., angiogenic growth factors), these proteins are released by the tumor into nearby tissues, thereby stimulating new blood vessels to sprout from existing healthy blood vessels toward and into the tumor. Once new blood vessels enter the tumor, the tumor can rapidly expand its size and invade local tissue and organs. Through the new blood vessels, cancer cells may further escape into the circulation and lodge in other organs to form new tumors, also known as metastasis.

TF expression is observed in many types of cancer, including cervical cancer, and is associated with more aggressive disease. Furthermore, human TF also exists in a soluble alternatively-spliced form, asHTF. It has recently been found that asHTF promotes tumor growth (Hobbs et al., 2007, Thrombosis Res. 120(2):S13-S21).

Human cancers harbor numerous genetic and epigenetic alterations, generating neoantigens potentially recognizable by the immune system (Sjoblom et al., 2006, Science 314:268-74). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities. Until recently, cancer immunotherapy had focused substantial effort on approaches that enhance anti-tumor immune responses by adoptive-transfer of activated effector cells, immunization against relevant antigens, or providing non-specific immune-stimulatory agents such as cytokines. In the past decade, however, intensive efforts to develop specific immune checkpoint pathway inhibitors have begun to provide new immunotherapeutic approaches for treating cancer, including the development of an antibody, ipilimumab (YERVOY®), that binds to and inhibits CTLA-4 for the treatment of patients with advanced melanoma (Hodi et al., 2010, N Engl J Med 363:711-23) and the development of antibodies, such as nivolumab, cemiplimab and pembrolizumab, that bind specifically to the Programmed Death-1 (PD-1) receptor and block the inhibitory PD-1/PD-1 ligand pathway. See, e.g., Topalian et al., N Engl J Med 366:2443-54 (2012a); Topalian et al., Curr Opin Immunol 24:207-12 (2012b); Topalian et al., J Clin Oncol 32(10):1020-30 (2014); Hamid et al., N Engl J Med 369:134-144 (2013); Hamid and Carvajal, Expert Opin Biol Ther 13(6):847-61 (2013); and McDermott and Atkins, Cancer Med 2(5):662-73 (2013).

Breast cancer is by far the most common cancer among women, Each year, more than 180,000 and 1 million women in the U.S. and worldwide, respectively, are diagnosed with breast cancer. Breast cancer is the leading cause of death for women between ages 50-55, and is the most common non-preventable malignancy in women in the Western Hemisphere. An estimated 2,167,000 women in the United States are currently living with the disease (National Cancer Institute, Surveillance Epidemiology and End Results (NCI SEER) program, Cancer Statistics Review (CSR), www-seer.ims.nci.nih.gov/Publications/CSR1973 (1998)). Based on cancer rates from 1995 through 1997, a report from the National Cancer Institute (NCI) estimates that about 1 in 8 women in the United States (approximately 12.8 percent) will develop breast cancer during her lifetime (NCIs Surveillance, Epidemiology, and End Results Program (SEER) publication SEER Cancer Statistic's Review 1973-1997). Breast cancer is the second most common form of cancer, after skin cancer, among women in the United States. An estimated 250,100 new cases of breast cancer are expected to be diagnosed in the United States in 2001. Of these, 192,200 new cases of more advanced (invasive) breast cancer are expected to occur among women (an increase of 5% over last year), 46,400 new cases of early stage (in situ) breast cancer are expected to occur among women (up 9% from last year), and about 1,500 new cases of breast cancer are expected to be diagnosed in men (Cancer Facts & FIGS. 2001 American Cancer Society). An estimated 40,600 deaths (40,300 women, 400 men) from breast cancer are expected in 2001. Breast cancer ranks second only to lung cancer among causes of cancer deaths in women. Nearly 86% of women who are diagnosed with breast cancer are likely to still be alive five years later, though 24% of them will die of breast cancer after 10 years, and nearly half (47%) will die of breast cancer after 20 years.

Every woman is at risk for breast cancer. Over 70 percent of breast cancers occur in women who have no identifiable risk factors other than age (U.S. General Accounting Office. Breast Cancer, 1971-1991: Prevention, Treatment and Research. GAO/PEMD-92-12; 1991). Only 5 to 10% of breast cancers are linked to a family history of breast cancer (Henderson I C, Breast Cancer. In: Murphy G P, Lawrence W L, Lenhard R E (eds). Clinical Oncology. Atlanta, Ga.: American Cancer Society; 1995:198-219).

Cervical cancer poses a significant medical problem worldwide with an estimated incidence of more than 500,000 new cases and 250,000 deaths annually. See Tewari et al., 2014, N Engl J Med., 370:734-743. In the Europe Union, approximately 34,000 new cases of cervical cancer and 13,000 deaths occur annually. See Hillemanns et al., 2016, Oncol. Res. Treat. 39:501-506. The main types of cervical cancer are squamous cell carcinoma and adenocarcinoma. Long-lasting infections with human papillomavirus (HPV) type 16 and 18 cause most cases of cervical cancer. The standard for first-line therapy of cervical cancer was a platinum-based therapy plus a taxane-based therapy. Bevacizumab, an anti-VEGF antibody, was approved by the U.S. Food and Drug Administration for use in combination with chemotherapy for the treatment of cervical cancer, which had improved overall survival in clinical trials. First-line (1L) treatment for advanced cervical cancer is comprised of bevacizumab combined with paclitaxel plus a platinum (e.g., cisplatin or carboplatin) or paclitaxel plus topotecan. Despite a 48% objective response rate (ORR) and a median overall survival (OS) of approximately 18 months, unfortunately almost all patients relapse after this 1L treatment. See Tewari et al., 2014, N Engl J Med., 370:734-743. For second-line (2L) treatment, no approved therapy is available and patients are often treated with single agent modalities including, but not limited to: pemetrexed, topotecan, docetaxel, nab-paclitaxel, vinorelbine and in some cases bevacizumab. A meta-analysis of single agent treatment demonstrates a modest response rate of only 10.9% (i.e., 60 responders out of 552 patients) and median overall survivals (OS) of approximately 7 months. See e.g., Burotto et al., 2015, Oncologist 20:725-726; Candelaria et al., 2009, Int. J. Gynecol. Cancer. 19:1632-1637; Coronel et al., 2009, Med. Oncol. 26:210-214; Fiorica et al., 2009, Gynecol. Oncol. 115:285-289; Garcia et. al., 2007, Am. J. Clin. Oncol. 30-428-431; Goncalves et al., 2008, Gynecol. Oncol. 108:42-46; Homesley et al., 2008, Int. J. Clin. Oncol. 13:62-65; McLachlan et al., 2017, Clin. Oncol. (R. Coll. Radiol.) 29:153-160; Miller et al., 2008, Gynecol. Oncol. 110:65-70; Monk et al., 2009, J. Clin. Oncol. 27:1069-1074; Muggia et al., 2004, Gynecol. Oncol. 92:639-643; Rose et al., 2006, Gynecol. Oncol. 102:210-213; Santin et al., 2011, Gynecol. Oncol. 122:495-500; Schilder et al., 2005, Gynecol. Oncol. 96:103-107; and Torfs et al., 2012, Eur. J. Cancer. 48:1332-1340. The five year relative survival for stage IV cervical cancer is only 15%, demonstrating a high need for improved therapy against cervical cancer.

Targeted therapy of multiple non-redundant molecular pathways regulating immune responses can enhance antitumor immunotherapy. However, not all combinations have acceptable safety and/or efficacy. There remains a need for combination therapies with an acceptable safety profile and high efficacy for the treatment of cancer, in particular for the treatment of breast cancer and cervical cancer.

All references cited herein, including patent applications, patent publications, and scientific literature, are herein incorporated by reference in their entirety, as if each individual reference were specifically and individually indicated to be incorporated by reference.

### SUMMARY

Provided herein are methods of treating cancer in a subject comprising administering to the subject an anti-PD-1 antibody or an antigen-binding fragment thereof, wherein the antibody binds to Programmed Death-1 (PD-1) and inhibits PD-1 activity, wherein the anti-PD-1 antibody or antigen-binding fragment thereof comprises the complementary determining regions (CDRs) of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof, and an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof. In some embodiments, the anti-PD-1 antibody or antigen-binding fragment thereof comprises the CDRs of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some of any of the embodiments herein, the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some of any of the embodiments herein, the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some of any of the embodiments herein, the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some of any of the embodiments herein, the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some of any of the embodiments herein, the antibody-drug conjugate is administered at a dose ranging from about 0.9 mg/kg to about 2.1 mg/kg. In some embodiments, the antibody-drug conjugate is administered at a dose of about 2.0 mg/kg. In some embodiments, the antibody-drug conjugate is administered at a dose of 2.0 mg/kg. In some of any of the embodiments herein, the antibody-drug conjugate is administered once about every 1 week, once about every 2 weeks, once about every 3 weeks or once about every 4 weeks. In some embodiments, the antibody-drug conjugate is administered once about every 3 weeks. In some of any of the embodiments herein, the anti-PD-1 antibody or antigen-binding fragment thereof comprises a heavy chain variable region and a light chain variable region, wherein the heavy chain variable region comprises:
(i) a CDR-H1 comprising the amino acid sequence of SEQ ID NO:17;
(ii) a CDR-H2 comprising the amino acid sequence of SEQ ID NO:18; and
(iii) a CDR-H3 comprising the amino acid sequence of SEQ ID NO:19; and wherein the light chain variable region comprises:
(i) a CDR-L1 comprising the amino acid sequence of SEQ ID NO:20;
(ii) a CDR-L2 comprising the amino acid sequence of SEQ ID NO:21; and
(iii) a CDR-L3 comprising the amino acid sequence of SEQ ID NO:22. In some of any of the embodiments herein, the anti-PD-1 antibody or antigen-binding fragment thereof comprises a heavy chain variable region comprising an amino acid sequence having at least 85% sequence identity to the amino acid sequence of SEQ ID NO:31 and a light chain variable region comprising an amino acid sequence having at least 85% sequence identity to the amino acid sequence of SEQ ID NO:32. In some of any of the embodiments herein, the anti-PD-1 antibody or antigen-binding fragment thereof comprises a heavy chain variable region comprising the amino acid sequence of SEQ ID NO:31 and a light chain variable region comprising the amino acid sequence of SEQ ID NO:32. In some of any of the embodiments herein, the anti-PD-1 antibody or antigen-binding fragment thereof is nivolumab. In some of any of the embodiments herein, the anti-PD-1 antibody or antigen-binding fragment thereof is administered at a dose ranging from about 0.5 mg/kg to about 4.1 mg/kg. In some of any of the embodiments herein, the anti-PD-1 antibody or antigen-binding fragment thereof is administered at a flat dose ranging from about 50 mg to about 500 mg. In some of any of the embodiments herein, the anti-PD-1 antibody or antigen-binding fragment thereof is administered at a flat dose of about 240 mg. In some of any of the embodiments herein, the anti-PD-1 antibody or antigen-binding fragment thereof is administered at a flat dose of about 480 mg. In some of any of the embodiments herein, the anti-PD-1 antibody or antigen-binding fragment thereof is administered once about every 1 week, once about every 2 weeks, once about every 3 weeks or once about every 4 weeks. In some embodiments, the anti-PD-1 antibody or antigen-binding fragment thereof is administered once about every 2 weeks. In some of any of the embodiments herein, the cancer is breast cancer. In some of any of the embodiments herein, the cancer is cervical cancer. In some of any of the embodiments herein, the subject is not a candidate for curative therapy. In some embodiments, curative therapy comprises radiotherapy and/or exenterative surgery. In some of any of the embodiments herein, the subject has not received prior systemic therapy for the cervical cancer. In some of any of the embodiments herein, the cervical cancer is an adenocarcinoma, an adenosquamous carcinoma or a squamous cell carcinoma. In some of any of the embodiments herein, the cervical cancer is an advanced stage cervical cancer. In some embodiments, the advanced stage cervical cancer is a stage 3 or stage 4 cervical cancer. In some of any of the embodiments herein, the advanced stage cervical cancer is metastatic cervical cancer. In some of any of the embodiments herein, the cervical cancer is recurrent cervical cancer. In some of any of the embodiments herein, the monomethyl auristatin is monomethyl auristatin E (MMAE). In some of any of the embodiments herein, the anti-TF antibody or antigen-binding fragment thereof of the antibody-drug conjugate is a monoclonal antibody or a monoclonal antigen-binding fragment thereof. In some of any of the embodiments herein, the anti-TF antibody or antigen-binding fragment thereof of the antibody-drug conjugate comprises a heavy chain variable region and a light chain variable region, wherein the heavy chain variable region comprises:
(i) a CDR-H1 comprising the amino acid sequence of SEQ ID NO:1;
(ii) a CDR-H2 comprising the amino acid sequence of SEQ ID NO:2; and
(iii) a CDR-H3 comprising the amino acid sequence of SEQ ID NO:3; and wherein the light chain variable region comprises:
(i) a CDR-L1 comprising the amino acid sequence of SEQ ID NO:4;
(ii) a CDR-L2 comprising the amino acid sequence of SEQ ID NO:5; and
(iii) a CDR-L3 comprising the amino acid sequence of SEQ ID NO:6. In some of any of the embodiments herein, the anti-TF antibody or antigen-binding fragment thereof of the antibody-drug conjugate comprises a heavy chain variable region comprising an amino acid sequence having at least 85% sequence identity to the amino acid sequence of SEQ ID NO:7 and a light chain variable region comprising an amino acid sequence having at least 85% sequence identity to the amino acid sequence of SEQ ID NO:8. In some of any of the embodiments herein, the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8. In some of any of the embodiments herein, the anti-TF antibody of the antibody-drug conjugate is tisotumab. In some of any of the embodiments herein, the antibody-drug conjugate further comprises a linker between the anti-TF antibody or antigen-binding fragment thereof and the monomethyl auristatin. In some embodiments, the linker is a cleavable peptide linker. In some embodiments, the cleavable peptide linker has a formula: -MC-vc-PAB-, wherein:
a) MC is:

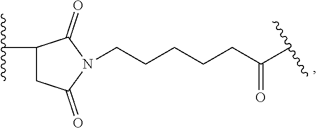 [SMILES: *C(=O)CCCCCN1C(=O)CC(*)C1=O]

b) vc is the dipeptide valine-citrulline, and
c) PAB is:

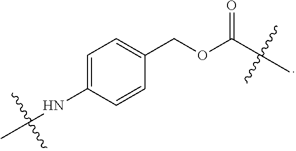 [SMILES: *Nc1ccc(COC(*)=O)cc1]

In some of any of the embodiments herein, the linker is attached to sulphydryl residues of the anti-TF antibody obtained by partial reduction or full reduction of the anti-TF antibody or antigen-binding fragment thereof. In some embodiments, the linker is attached to MMAE (vcMMAE), wherein the antibody-drug conjugate has the following structure:

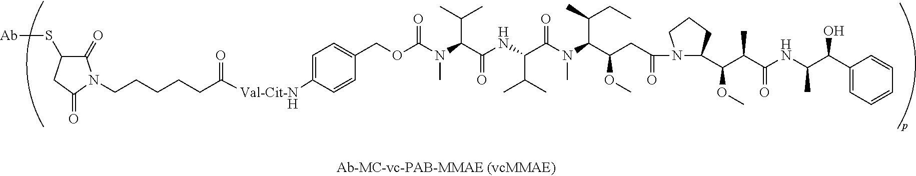

wherein p denotes a number from 1 to 8, S represents a sulphydryl residue of the anti-TF antibody, and Ab designates the anti-TF antibody or antigen-binding fragment thereof. In some of any of the embodiments herein, the average value of p in a population of the antibody-drug conjugates is about 4. In some of any of the embodiments herein, the antibody-drug conjugate is tisotumab vedotin. In some of any of the embodiments herein, the route of administration for the antibody-drug conjugate is intravenous. In some of any of the embodiments herein, the route of administration for the anti-PD-1 antibody or antigen-binding fragment thereof is intravenous. In some of any of the embodiments herein, the anti-PD-1 antibody or antigen-binding fragment thereof and the antibody-drug conjugate are administered sequentially. In some of any of the embodiments herein, the anti-PD-1 antibody or antigen-binding fragment thereof and the antibody-drug conjugate are administered simultaneously. In some of any of the embodiments herein, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express TF. In some of any of the embodiments herein, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express PD-L1. In some of any of the embodiments herein, a tumor derived from the cancer comprises one or more cells that express PD-L1, PD-L2, or both PD-L1 and PD-L2. In some of any of the embodiments herein, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express PD-1. In some of any of the embodiments herein, one or more therapeutic effects in the subject is improved after administration of the antibody-drug conjugate and the anti-PD-1 antibody or antigen-binding fragment thereof relative to a baseline. In some embodiments, the one or more therapeutic effects is selected from the group consisting of: size of a tumor derived from the cancer, objective response rate, duration of response, time to response, progression free survival, and overall survival. In some of any of the embodiments herein, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody-drug conjugate and the anti-PD-1 antibody or antigen-binding fragment thereof. In some of any of the embodiments herein, the objective response rate is at least about 20%, at least about 25%, at least about 30%, at least about 35%, at least about 40%, at least about 45%, at least about 50%, at least about 60%, at least about 70%, or at least about 80%. In some of any of the embodiments herein, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof. In some of any of the embodiments herein, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof. In some of any of the embodiments herein, the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof. In some of any of the embodiments herein, the subject has one or more adverse events and is further administered an additional therapeutic agent to eliminate or reduce the severity of the one or more adverse events. In some of any of the embodiments herein, the subject is at risk of developing one or more adverse events and is further administered an additional therapeutic agent to prevent or reduce the severity of the one or more adverse events. In some of any of the embodiments herein, the one or more adverse events is anemia, abdominal pain, hemorrhage, hyperthyroidism, hypothyroidism, hypokalemia, hyponatremia, epistaxis, fatigue, nausea, alopecia, conjunctivitis, keratitis, conjunctival ulceration, constipation, decreased appetite, diarrhea, vomiting, peripheral neuropathy, or general physical health deterioration. In some of any of the embodiments herein, the one or more adverse events is a grade 3 or greater adverse event. In some of any of the embodiments herein, the one or more adverse events is a serious adverse event. In some of any of the embodiments herein, the one or more adverse events is conjunctivitis, conjunctival ulceration, and/or keratitis and the additional agent is a preservative-free lubricating eye drop, an ocular vasoconstrictor, antibiotic, and/or a steroid eye drop. In some of any of the embodiments herein, the subject is a human. In some of any of the embodiments herein, the antibody-drug conjugate is in a pharmaceutical composition comprising the antibody-drug conjugate and a pharmaceutical acceptable carrier. In some of any of the embodiments herein, the anti-PD-1 antibody or antigen-binding fragment thereof is in a pharmaceutical composition comprising the anti-PD-1 antibody or antigen-binding fragment thereof and a pharmaceutical acceptable carrier.

Also provided herein are kits comprising:
(a) an antibody or an antigen-binding fragment thereof described herein, wherein the antibody binds to Programmed Death-1 (PD-1) and inhibits PD-1 activity;
(b) a dosage ranging from about 0.9 mg/kg to about 2.1 mg/kg of an antibody-drug conjugate described herein that binds to tissue factor (TF), wherein the antibody-drug conjugate comprises an anti-TF antibody or an antigen-binding fragment thereof conjugated to a monomethyl auristatin or a functional analog thereof or a functional derivative thereof; and
(c) instructions for use of the anti-PD-1 antibody or antigen-binding fragment thereof and the antibody drug conjugate according to some of any of the embodiments herein.

### BRIEF DESCRIPTION OF THE DRAWINGS

FIG. 1 is an image of a Western blot showing phosphorylation of IRE1 and JNK in cell lysates of HeLa cells treated with MMAE (right lane) as compared to HeLa cells not treated with MMAE (left lane). Treatment with MMAE led to phosphorylation of both IRE1 and JNK. pIRE1 indicates phosphorylated IRE1 protein; IRE1 indicated total IRE1 protein; and pJNK indicates phosphorylated JNK protein.

FIGS. 2A and 2B are immunofluorescent images of HeLa cells treated with 100 nM MMAE and imaged at the indicated time points in the presence of MMAE. A) Top panel shows staining for the ER with the ER-binding dye ER-ID Green and B) the lower panel shows RFP-labeled tubulin expressed by the cells

FIGS. 3A and 3B is a series of graphs showing A) ATP secretion and B) HMGB1 secretion from HeLa cells treated with 100 nM MMAE as compared to HeLa cells not treated with MMAE. Measurements were treated HeLa cells are shown as the fold change over the signal produced by untreated HeLa cells. **p<0.01 and ****p<0.0001.

FIG. 4A-4C is a series of graphs showing that tisotumab vedotin antibody-drug conjugate and MMAE free drug both drove robust A) ATP secretion and C) HMGB1 release. Activity was specific to the targeted agent (tisotumab vedotin) and free drug (MMAE). The non-targeted isotype ADC (IgG1-MMAE) did not elicit A) ATP or C) HMGB1 secretion. B) Tisotumab vedotin was active on multiple Tissue Factor positive cell lines.

FIG. 5 is images of a Western blots showing treatment of HPAFII (pancreatic carcinoma) or MDA-MB-231 (breast carcinoma) cells for 16 hours with tisotumab vedotin or MMAE payloads trigger multiple ER stress pathways including phosphorylation of IRE and its down-stream target JNK as well as cleavage of ATF4. Treatment with the non-targeting H00-MMAE ADC (IgG1 MMAE) did not trigger activation of these ER stress pathways.

FIGS. 6A and 6B is a series of graphs in which Tissue Factor positive MDA-MB-231 cells killed with various agents were fed to human peripheral blood mononuclear cells (PBMCs) and immune activation assessed by increased expression of activations markers on innate CD14+ monocyte/macrophages and induction of chemokine and cytokine production. Treatment with tisotumab vedotin ADC or MMAE free drug drove monocyte/macrophage activations as monitored by A) CD86 expression by flow cytometry and B) induced release of innate chemokines including MIP10 compared to non-targeting IgG1-MMAE ADC or targeting antibody (tisotumab) alone.

FIG. 7A-7C is a series of graphs in which Tissue Factor positive MDA-MB-231 cells killed with various agents were fed to CSFE labeled human peripheral blood mononuclear cells (PBMCs) in the presence or absence of the PD1 targeting antibody nivolumab for 48 hours and T cells activation assessed by A) decreased CSFE fluorescent indicative of T cell proliferation and B) and C) cytokine production. Treatment with tisotumab vedotin or MMAE free drug drove T cell proliferation, which was enhanced with 2 mg/ml of nivolumab treatment. Production of B) IL12p70 and C) IFN7 was also increased following exposure to tisotumab vedotin and MMAE killed cell and cytokine production was increased by concomitant nivolumab treatment.

### DETAILED DESCRIPTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that aspects and embodiments of the invention described herein include “comprising,” “consisting,” and “consisting essentially of” aspects and embodiments.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The terms “tissue factor”, “TF”, “CD142”, “tissue factor antigen”, “TF antigen” and “CD142 antigen” are used interchangeably herein, and, unless specified otherwise, include any variants, isoforms and species homologs of human tissue factor which are naturally expressed by cells or are expressed on cells transfected with the tissue factor gene. In some embodiments, tissue factor comprises the amino acid sequence found under Genbank accession NP_001984.

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as V_{H} or VH) and a heavy chain constant region (C_{H} or CH). The heavy chain constant region typically is comprised of three domains, C_{H}1, C_{H}2, and C_{H}3. The heavy chains are generally inter-connected via disulfide bonds in the so-called “hinge region.” Each light chain typically is comprised of a light chain variable region (abbreviated herein as V_{L} or VL) and a light chain constant region (C_{L} or CL). The light chain constant region typically is comprised of one domain, C_{L}. The CL can be of κ (kappa) or κ (lambda) isotype. The terms “constant domain” and “constant region” are used interchangeably herein. An immunoglobulin can derive from any of the commonly known isotypes, including but not limited to IgA, secretory IgA, IgG, and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes.

The term “variable region” or “variable domain” refers to the domain of an antibody heavy or light chain that is involved in binding the antibody to antigen. The variable regions of the heavy chain and light chain (V_{H} and V_{L}, respectively) of a native antibody may be further subdivided into regions of hypervariability (or hypervariable regions, which may be hypervariable in sequence and/or form of structurally defined loops), also termed complementarity-determining regions (CDRs), interspersed with regions that are more conserved, termed framework regions (FRs). The terms “complementarity determining regions” and “CDRs,” synonymous with “hypervariable regions” or “HVRs” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). Within each VH and V_{L}, three CDRs and four FRs are typically arranged from amino-terminus to carboxy-terminus in the following order: FR1, CDR1, FR2, CDR2, FR3, CDR3, FR4 (See also Chothia and Lesk J. Mol. Biol., 195, 901-917 (1987)).

The term “antibody” (Ab) in the context of the present invention refers to an immunoglobulin molecule, a fragment of an immunoglobulin molecule, or a derivative of either thereof, which has the ability to specifically bind to an antigen under typical physiological conditions with a half-life of significant periods of time, such as at least about 30 min, at least about 45 min, at least about one hour (h), at least about two hours, at least about four hours, at least about eight hours, at least about 12 hours (h), about 24 hours or more, about 48 hours or more, about three, four, five, six, seven or more days, etc., or any other relevant functionally-defined period (such as a time sufficient to induce, promote, enhance, and/or modulate a physiological response associated with antibody binding to the antigen and/or time sufficient for the antibody to recruit an effector activity). The variable regions of the heavy and light chains of the immunoglobulin molecule contain a binding domain that interacts with an antigen. The constant regions of the antibodies (Abs) may mediate the binding of the immunoglobulin to host tissues or factors, including various cells of the immune system (such as effector cells) and components of the complement system such as Clq, the first component in the classical pathway of complement activation. An antibody may also be a bispecific antibody, diabody, multispecific antibody or similar molecule.

The term “monoclonal antibody” as used herein refers to a preparation of antibody molecules that are recombinantly produced with a single primary amino acid sequence. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be generated by a hybridoma which includes a B cell obtained from a transgenic or transchromosomal non-human animal, such as a transgenic mouse, having a genome comprising a human heavy chain transgene and a light chain transgene, fused to an immortalized cell.

An “isolated antibody” refers to an antibody that is substantially free of other antibodies having different antigenic specificities (e.g., an isolated antibody that binds specifically to TF is substantially free of antibodies that bind specifically to antigens other than TF). An isolated antibody that binds specifically to TF can, however, have cross-reactivity to other antigens, such as TF molecules from different species. Moreover, an isolated antibody can be substantially free of other cellular material and/or chemicals. In one embodiment, an isolated antibody includes an antibody conjugate attached to another agent (e.g., small molecule drug). In some embodiments, an isolated anti-TF antibody includes a conjugate of an anti-TF antibody with a small molecule drug (e.g., MMAE or MMAF).

A “human antibody” (HuMAb) refers to an antibody having variable regions in which both the FRs and CDRs are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibodies” and “fully human antibodies” and are used synonymously.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “chimeric antibody” as used herein, refers to an antibody wherein the variable region is derived from a non-human species (e.g. derived from rodents) and the constant region is derived from a different species, such as human. Chimeric antibodies may be generated by antibody engineering. “Antibody engineering” is a term used generic for different kinds of modifications of antibodies, and which is a well-known process for the skilled person. In particular, a chimeric antibody may be generated by using standard DNA techniques as described in Sambrook et al., 1989, Molecular Cloning: A laboratory Manual, New York: Cold Spring Harbor Laboratory Press, Ch. 15. Thus, the chimeric antibody may be a genetically or an enzymatically engineered recombinant antibody. It is within the knowledge of the skilled person to generate a chimeric antibody, and thus, generation of the chimeric antibody according to the present invention may be performed by other methods than described herein. Chimeric monoclonal antibodies for therapeutic applications are developed to reduce antibody immunogenicity. They may typically contain non-human (e.g. murine) variable regions, which are specific for the antigen of interest, and human constant antibody heavy and light chain domains. The terms “variable region” or “variable domains” as used in the context of chimeric antibodies, refers to a region which comprises the CDRs and framework regions of both the heavy and light chains of the immunoglobulin.

An “anti-antigen antibody” refers to an antibody that binds to the antigen. For example, an anti-TF antibody is an antibody that binds to the antigen TF. In another example, an anti-PD-1 antibody is an antibody that binds to the antigen PD-1.

An “antigen-binding portion” or antigen-binding fragment” of an antibody refers to one or more fragments of an antibody that retain the ability to bind specifically to the antigen bound by the whole antibody. Examples of antibody fragments (e.g., antigen-binding fragment) include but are not limited to Fv, Fab, Fab′, Fab′-SH, F(ab′)₂; diabodies; linear antibodies; single-chain antibody molecules (e.g. scFv); and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For example, the % sequence identity of a given amino acid sequence A to, with, or against a given amino acid sequence B (which can alternatively be phrased as a given amino acid sequence A that has or comprises a certain % sequence identity to, with, or against a given amino acid sequence B) is calculated as follows:
100 times the fraction X/Y
   where X is the number of amino acid residues scored as identical matches by the sequence in that program's alignment of A and B, and where Y is the total number of amino acid residues in B. It will be appreciated that where the length of amino acid sequence A is not equal to the length of amino acid sequence B, the % sequence identity of A to B will not equal the % sequence identity of B to A.

As used herein, the terms “binding”, “binds” or “specifically binds” in the context of the binding of an antibody to a pre-determined antigen typically is a binding with an affinity corresponding to a K_{D} of about 10⁻⁶ M or less, e.g. 10⁻⁷ M or less, such as about 10⁻⁸ M or less, such as about 10⁻⁹ M or less, about 10⁻¹⁰ M or less, or about 10⁻¹¹ M or even less when determined by for instance BioLayer Interferometry (BLI) technology in a Octet HTX instrument using the antibody as the ligand and the antigen as the analyte, and wherein the antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} of binding to a non-specific antigen (e.g., BSA, casein) other than the predetermined antigen or a closely related antigen. The amount with which the K_{D} of binding is lower is dependent on the K_{D} of the antibody, so that when the K_{D} of the antibody is very low, then the amount with which the K_{D} of binding to the antigen is lower than the K_{D} of binding to a non-specific antigen may be at least 10,000-fold (that is, the antibody is highly specific).

The term “K_{D}” (M), as used herein, refers to the dissociation equilibrium constant of a particular antibody-antigen interaction. Affinity, as used herein, and K_{D} are inversely related, that is that higher affinity is intended to refer to lower K_{D}, and lower affinity is intended to refer to higher K_{D}.

The term “ADC” refers to an antibody-drug conjugate, which in the context of the present invention refers to an anti-TF antibody, which is coupled to a drug moiety (e.g., MMAE or MMAF) as described in the present application.

The abbreviations “vc” and “val-cit” refer to the dipeptide valine-citrulline.

The abbreviation “PAB” refers to the self-immolative spacer:

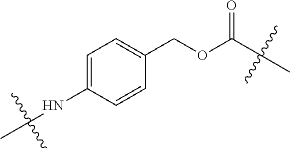 [SMILES: *Nc1ccc(COC(*)=O)cc1]

The abbreviation “MC” refers to the stretcher maleimidocaproyl:

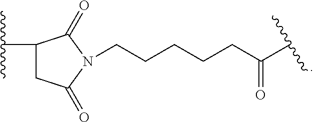 [SMILES: *C(=O)CCCCCN1C(=O)CC(*)C1=O]

The term “Ab-MC-vc-PAB-MMAE” refers to an antibody conjugated to the drug MMAE through a MC-vc-PAB linker.

“Programmed Death-1” (PD-1) refers to an immunoinhibitory receptor belonging to the CD28 family. PD-1 is expressed predominantly on previously activated T-cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. In some embodiments, hPD-1 comprises the amino acid sequence found under GenBank Accession No. U64863.

“Programmed Death Ligand-1” (PD-L1) is one of two cell surface glycoprotein ligands for PD-1 (the other being PD-L2) that downregulate T-cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. In some embodiments, hPD-L1 comprises the amino acid sequence found under GenBank Accession No. Q9NZQ7.

A “cancer” refers to a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor. For example, a “tumor derived from” a cervical cancer refers to a tumor that is the result of a metastasized cervical cancer.

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease. In some embodiments, the disease is cancer.

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

An “effective amount” or “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example for the treatment of tumors, a therapeutically effective amount of an anti-cancer agent inhibits cell growth or tumor growth by at least about 10%, by at least about 20%, by at least about 30%, by at least about 40%, by at least about 50%, by at least about 60%, by at least about 70%, or by at least about 80%, by at least about 90%, by at least about 95%, by at least about 96%, by at least about 97%, by at least about 98%, or by at least about 99% in a treated subject(s) (e.g., one or more treated subjects) relative to an untreated subject(s) (e.g., one or more untreated subjects). In some embodiments, a therapeutically effective amount of an anti-cancer agent inhibits cell growth or tumor growth by 100% in a treated subject(s) (e.g., one or more treated subjects) relative to an untreated subject(s) (e.g., one or more untreated subjects).

In other embodiments of the disclosure, tumor regression can be observed and continue for a period of at least about 20 days, at least about 30 days, at least about 40 days, at least about 50 days, or at least about 60 days. Notwithstanding these ultimate measurements of therapeutic effectiveness, evaluation of immunotherapeutic drugs must also make allowance for “immune-related response patterns”.

A therapeutically effective amount of a drug (e.g., anti-TF antibody-drug conjugate or anti-PD-1 antibody) includes a “prophylactically effective amount,” which is any amount of the drug that, when administered alone or in combination with an anti-cancer agent to a subject at risk of developing a cancer (e.g., a subject having a pre-malignant condition) or of suffering a recurrence of cancer, inhibits the development or recurrence of the cancer. In some embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

As used herein, “subtherapeutic dose” means a dose of a therapeutic compound (e.g., an anti-TF antibody-drug conjugate or anti-PD-1 antibody) that is lower than the usual or typical dose of the therapeutic compound when administered alone for the treatment of a hyperproliferative disease (e.g., cancer).

An “immune-related response pattern” refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents can require long-term monitoring of the effects of these agents on the target disease.

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration that is at least the same as the treatment duration, or at least 1.5, 2.0, 2.5, or 3 times longer than the treatment duration.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progression free survival” or “PFS” refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” or “ORR” refers to the sum of complete response (CR) rate and partial response (PR) rate.

As used herein, “overall survival” or “OS” refers to the percentage of individuals in a group who are likely to be alive after a particular duration of time.

The term “weight-based dose”, as referred to herein, means that a dose administered to a subject is calculated based on the weight of the subject. For example, when a subject with 60 kg body weight requires 2.0 mg/kg of an anti-PD-1 antibody or an anti-TF antibody-drug conjugate, one can calculate and use the appropriate amount of the anti-PD-1 antibody or anti-TF antibody-drug conjugate (i.e., 120 mg) for administration to said subject.

The use of the term “fixed dose” with regard to a method of the disclosure means that two or more different antibodies (e.g., anti-PD-1 antibody and anti-TF antibody-drug conjugate) are administered to a subject in particular (fixed) ratios with each other. In some embodiments, the fixed dose is based on the amount (e.g., mg) of the antibodies. In certain embodiments, the fixed dose is based on the concentration (e.g., mg/ml) of the antibodies. For example, a 3:1 ratio of an anti-PD-1 antibody to an anti-TF antibody-drug conjugate administered to a subject can mean about 240 mg of the anti-PD-1 antibody and about 80 mg of the anti-TF antibody-drug conjugate or about 3 mg/ml of the anti-PD-1 antibody and about 1 mg/ml of the anti-TF antibody-drug conjugate are administered to the subject.

The use of the term “flat dose” with regard to the methods and dosages of the disclosure means a dose that is administered to a subject without regard for the weight or body surface area (BSA) of the subject. The flat dose is therefore not provided as a mg/kg dose, but rather as an absolute amount of the agent (e.g., the anti-TF antibody-drug conjugate and/or anti-PD-1 antibody). For example, a subject with 60 kg body weight and a subject with 100 kg body weight would receive the same dose of an antibody or an antibody-drug conjugate (e.g., 240 mg of an anti-TF antibody-drug conjugate or e.g. 240 mg of an anti-PD-1 antibody).

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “resylate”, ethanesulfonate, benzenesulfonate, p-toluenesulfonate, pamoate (i.e., 4,4′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Administering” or “administration” refer to the physical introduction of a therapeutic agent to a subject, using any of the various methods and delivery systems known to those skilled in the art. Exemplary routes of administration for the anti-TF antibody-drug conjugate and/or anti-PD-1 antibody include intravenous, intramuscular, subcutaneous, intraperitoneal, spinal or other parenteral routes of administration, for example by injection or infusion (e.g., intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administration can also be performed, for example, once, a plurality of times, and/or over one or more extended periods.

The terms “baseline” or “baseline value” used interchangeably herein can refer to a measurement or characterization of a symptom before the administration of the therapy (e.g., an anti-TF antibody-drug conjugate as described herein and/or an anti-PD-1 antibody as described herein) or at the beginning of administration of the therapy. The baseline value can be compared to a reference value in order to determine the reduction or improvement of a symptom of a TF-associated disease and/or PD-1 associated disease contemplated herein (e.g., breast cancer or cervical cancer). The terms “reference” or “reference value” used interchangeably herein can refer to a measurement or characterization of a symptom after administration of the therapy (e.g., an anti-TF antibody-drug conjugate as described herein and/or an anti-PD-1 antibody as described herein). The reference value can be measured one or more times during a dosage regimen or treatment cycle or at the completion of the dosage regimen or treatment cycle. A “reference value” can be an absolute value; a relative value; a value that has an upper and/or lower limit; a range of values; an average value; a median value: a mean value; or a value as compared to a baseline value.

Similarly, a “baseline value” can be an absolute value; a relative value; a value that has an upper and/or lower limit; a range of values; an average value; a median value; a mean value; or a value as compared to a reference value. The reference value and/or baseline value can be obtained from one individual, from two different individuals or from a group of individuals (e.g., a group of two, three, four, five or more individuals).

The term “monotherapy” as used herein means that the anti-TF antibody-drug conjugate or anti-PD-1 antibody is the only anti-cancer agent administered to the subject during the treatment cycle. Other therapeutic agents, however, can be administered to the subject. For example, anti-inflammatory agents or other agents administered to a subject with cancer to treat symptoms associated with cancer, but not the underlying cancer itself, including, for example inflammation, pain, weight loss, and general malaise, can be administered during the period of monotherapy.

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

A “serious adverse event” or “SAE” as used herein is an adverse event that meets one of the following criteria:
Is fatal or life-threatening (as used in the definition of a serious adverse event, “life-threatening” refers to an event in which the patient was at risk of death at the time of the event; it does not refer to an event which hypothetically might have caused death if it was more severe.
Results in persistent or significant disability/incapacity
Constitutes a congenital anomaly/birth defect
Is medically significant, i.e., defined as an event that jeopardizes the patient or may require medical or surgical intervention to prevent one of the outcomes listed above. Medical and scientific judgment must be exercised in deciding whether an AE is “medically significant”
Requires inpatient hospitalization or prolongation of existing hospitalization, excluding the following: 1) routine treatment or monitoring of the underlying disease, not associated with any deterioration in condition; 2) elective or pre-planned treatment for a pre-existing condition that is unrelated to the indication under study and has not worsened since signing the informed consent; and 3) social reasons and respite care in the absence of any deterioration in the patient's general condition.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

The terms “once about every week,” “once about every two weeks,” or any other similar dosing interval terms as used herein mean approximate numbers. “Once about every week” can include every seven days ±one day, i.e., every six days to every eight days. “Once about every two weeks” can include every fourteen days ±two days, i.e., every twelve days to every sixteen days. “Once about every three weeks” can include every twenty-one days ±three days, i.e., every eighteen days to every twenty-four days. Similar approximations apply, for example, to once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Combination Therapy

One aspect of the invention provides anti-TF antibody-drug conjugates that binds to TF for use in the treatment of cancer wherein the antibody-drug conjugate is for administration, or to be administered in combination with an anti-PD-1 antibody or an antigen-binding fragment thereof wherein the antibody-drug conjugate comprises an anti-TF antibody or an antigen-binding fragment thereof conjugated to a monomethyl auristatin or a functional analog thereof or a functional derivative thereof, and wherein the anti-PD-1 antibody or antigen-binding fragment thereof comprises the complementary determining regions (CDRs) of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof comprises the CDRs of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the cancer is breast cancer. In some embodiments, the cancer is cervical cancer. In some embodiments, the cervical cancer is an advanced stage cervical cancer (e.g., stage 3 cervical cancer or stage 4 cervical cancer or metastatic cervical cancer). In some embodiments, the advanced cervical cancer is a metastatic cancer. In some embodiments, the subject has relapsed, recurrent and/or metastatic cervical cancer. In another aspect the present invention provides an anti-PD-1 antibody or an antigen-binding fragment thereof for use in the treatment of cancer wherein the anti-PD-1 antibody or antigen-binding fragment thereof is for administration, or to be administered in combination with an antibody-drug conjugate that binds to TF, wherein the antibody-drug conjugate comprises an anti-TF antibody or an antigen-binding fragment thereof conjugated to a monomethyl auristatin or a functional analog thereof or a functional derivative thereof, wherein the anti-PD-1 antibody or the antigen-binding fragment thereof inhibits PD-1 activity, and wherein the anti-PD-1 antibody or antigen-binding fragment thereof comprises the complementary determining regions (CDRs) of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof comprises the CDRs of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the cancer is breast cancer. In some embodiments, the cancer is cervical cancer. In some embodiments, the cervical cancer is an advanced stage cervical cancer (e.g., stage 3 cervical cancer or stage 4 cervical cancer or metastatic cervical cancer). In some embodiments, the advanced cervical cancer is a metastatic cancer. In some embodiments, the subject has relapsed, recurrent and/or metastatic cervical cancer.

### A. Anti-TF Antibody

Generally, anti-TF antibodies of the disclosure bind TF, e.g., human TF, and exert cytostatic and cytotoxic effects on malignant cells, such as breast cancer cells or cervical cancer cells. Anti-TF antibodies of the disclosure are preferably monoclonal, and may be multispecific, human, humanized or chimeric antibodies, single chain antibodies, Fab fragments, F(ab′) fragments, fragments produced by a Fab expression library, and TF binding fragments of any of the above. In some embodiments, the anti-TF antibodies of the disclosure specifically bind TF. The immunoglobulin molecules of the disclosure can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the disclosure, the anti-TF antibodies are antigen-binding fragments (e.g., human antigen-binding fragments) as described herein and include, but are not limited to, Fab, Fab′ and F(ab′)₂, Fd, single-chain Fvs (scFv), single-chain antibodies, disulfide-linked Fvs (sdFv) and fragments comprising either a V_{L} or VH domain. Antigen-binding fragments, including single-chain antibodies, may comprise the variable region(s) alone or in combination with the entirety or a portion of the following: hinge region, CH1, CH2, CH3 and CL domains. Also included in the present disclosure are antigen-binding fragments comprising any combination of variable region(s) with a hinge region, CH1, CH2, CH3 and CL domains. In some embodiments, the anti-TF antibodies or antigen-binding fragments thereof are human, murine (e.g., mouse and rat), donkey, sheep, rabbit, goat, guinea pig, camelid, horse, or chicken.

The anti-TF antibodies of the present disclosure may be monospecific, bispecific, trispecific or of greater multi specificity. Multispecific antibodies may be specific for different epitopes of TF or may be specific for both TF as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; Kostelny et al., 1992, J. Immunol. 148:1547 1553.

Anti-TF antibodies of the present disclosure may be described or specified in terms of the particular CDRs they comprise. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme). The boundaries of a given CDR may vary depending on the scheme used for identification. In some embodiments, a “CDR” or “complementarity determining region,” or individual specified CDRs (e.g., CDR-H1, CDR-H2, CDR-H3), of a given antibody or region thereof (e.g., variable region thereof) should be understood to encompass a (or the specific) CDR as defined by any of the aforementioned schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes. The scheme for identification of a particular CDR or CDRs may be specified, such as the CDR as defined by the Kabat, Chothia, AbM or IMGT method.

Numbering of amino acid residues in CDR sequences of the anti-TF antibodies of the anti-TF antibody-drug conjugates provided herein are according to the IMGT numbering scheme as described in Lefranc, M. P. et al., Dev. Comp. Immunol., 2003, 27, 55-77.

In certain embodiments antibodies of the disclosure comprise one or more CDRs of the antibody 011. See WO 2011/157741 and WO 2010/066803. The disclosure encompasses an antibody or derivative thereof comprising a heavy or light chain variable domain, said variable domain comprising (a) a set of three CDRs, in which said set of CDRs are from monoclonal antibody 011, and (b) a set of four framework regions, in which said set of framework regions differs from the set of framework regions in monoclonal antibody 011, and in which said antibody or derivative thereof binds to TF. In some embodiments, said antibody or derivative thereof specifically binds to TF. In certain embodiments, the anti-TF antibody is 011. The antibody 011 is also known as tisotumab.

In one aspect, anti-TF antibodies that compete with tisotumab binding to TF are also provided herein. Anti-TF antibodies that bind to the same epitope as tisotumab are also provided herein.

In one aspect, provided herein is an anti-TF antibody comprising 1, 2, 3, 4, 5, or 6 of the CDR sequences of tisotumab.

In one aspect, provided herein is an anti-TF antibody comprising a heavy chain variable region and a light chain variable region, wherein the heavy chain variable region comprises (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3; and/or wherein the light chain variable region comprises (i) CDR-L1 comprising the amino acid sequence of SEQ ID NO:4, (ii) CDR-L2 comprising the amino acid sequence of SEQ ID NO:5, and (iii) CDR-L3 comprising the amino acid sequence of SEQ ID NO:6.

An anti-TF antibody described herein may comprise any suitable framework variable domain sequence, provided that the antibody retains the ability to bind TF (e.g., human TF). As used herein, heavy chain framework regions are designated “HC-FR1-FR4,” and light chain framework regions are designated “LC-FR1-FR4.” In some embodiments, the anti-TF antibody comprises a heavy chain variable domain framework sequence of SEQ ID NO:9, 10, 11, and 12 (HC-FR1, HC-FR2, HC-FR3, and HC-FR4, respectively). In some embodiments, the anti-TF antibody comprises a light chain variable domain framework sequence of SEQ ID NO:13, 14, 15, and 16 (LC-FR1, LC-FR2, LC-FR3, and LC-FR4, respectively).

In some embodiments of the anti-TF antibodies described herein, the heavy chain variable domain comprises the amino acid sequence of EVQLLESGGGLVQPGGSLRLSCAASGFTFSNYAMSWVRQAPGKGLEWVSSISGSGD YTYYTDSVKGRFTISRDNSKNTLYLQMNSLRAEDTAVYYCARSPWGYYLDSWGQG TLVTVSS (SEQ ID NO:7) and the light chain variable domain comprises the amino acid sequence of

(SEQ ID NO: 8)
DIQMTQSPPSLSASAGDRVTITCRASQGISSRLAWYQQKPEKAPKSLIYA
ASSLQSGVPSRFSGSGSGTDFTLTISSLQPEDFATYYCQQYNSYPYTFGQ
GTKLEIK.

In some embodiments of the anti-TF antibodies described herein, the heavy chain CDR sequences comprise the following:
a) CDR-H1 (GFTFSNYA (SEQ ID NO:1));
b) CDR-H2 (ISGSGDYT (SEQ ID NO:2)); and
c) CDR-H3 (ARSPWGYYLDS (SEQ ID NO:3)).

a) CDR-H1
(GFTFSNYA (SEQ ID NO: 1));
b) CDR-H2
(ISGSGDYT (SEQ ID NO: 2));
and
c) CDR-H3
(ARSPWGYYLDS (SEQ ID NO: 3)).

In some embodiments of the anti-TF antibodies described herein, the heavy chain FR sequences comprise the following:

a) HC-FR1
(EVQLLESGGGLVQPGGSLRLSCAAS (SEQ ID NO: 9));
b) HC-FR2
(MSWVRQAPGKGLEWVSS (SEQ ID NO: 10));
c) HC-FR3
(YYTDSVKGRFTISRDNSKNTLYLQMNSLRAEDTAVYYC (SEQ
ID NO: 11));
and
d) HC-FR4
(WGQGTLVTVSS (SEQ ID NO: 12)).

In some embodiments of the anti-TF antibodies described herein, the light chain CDR sequences comprise the following:

a) CDR-L1
(QGISSR (SEQ ID NO: 4));
b) CDR-L2
(AAS (SEQ ID NO: 5));
and
c) CDR-L3
(QQYNSYPYT (SEQ ID NO: 6)).

In some embodiments of the anti-TF antibodies described herein, the light chain FR sequences comprise the following:

a) LC-FR1
(DIQMTQSPPSLSASAGDRVTITCRAS (SEQ ID NO: 13));
b) LC-FR2
(LAWYQQKPEKAPKSLIY (SEQ ID NO: 14));
c) LC-FR3
(SLQSGVPSRFSGSGSGTDFTLTISSLQPEDFATYYC (SEQ
ID NO: 15));
and
d) LC-FR4
(FGQGTKLEIK (SEQ ID NO: 16)).

a) LC-FR1 (DIQMTQSPPSLSASAGDRVTITCRAS (SEQ ID NO:13));
b) LC-FR2 (LAWYQQKPEKAPKSLIY (SEQ ID NO:14));
c) LC-FR3 (SLQSGVPSRFSGSGSGTDFTLTISSLQPEDFATYYC (SEQ ID NO:15)); and
d) LC-FR4 (FGQGTKLEIK (SEQ ID NO:16)).

In some embodiments, provided herein is an anti-TF antibody that binds to TF (e.g., human TF), wherein the antibody comprises a heavy chain variable region and a light chain variable region, wherein the antibody comprises:
(a) heavy chain variable domain comprising:
   (1) an HC-FR1 comprising the amino acid sequence of SEQ ID NO:9;
   (2) an CDR-H1 comprising the amino acid sequence of SEQ ID NO:1;
   (3) an HC-FR2 comprising the amino acid sequence of SEQ ID NO:10;
   (4) an CDR-H2 comprising the amino acid sequence of SEQ ID NO:2;
   (5) an HC-FR3 comprising the amino acid sequence of SEQ ID NO:11;
   (6) an CDR-H3 comprising the amino acid sequence of SEQ ID NO:3; and
   (7) an HC-FR4 comprising the amino acid sequence of SEQ ID NO:12, and/or
(b) a light chain variable domain comprising:
   (1) an LC-FR1 comprising the amino acid sequence of SEQ ID NO:13;
   (2) an CDR-L1 comprising the amino acid sequence of SEQ ID NO:4;
   (3) an LC-FR2 comprising the amino acid sequence of SEQ ID NO:14;
   (4) an CDR-L2 comprising the amino acid sequence of SEQ ID NO:5;
   (5) an LC-FR3 comprising the amino acid sequence of SEQ ID NO:15;
   (6) an CDR-L3 comprising the amino acid sequence of SEQ ID NO:6; and
   (7) an LC-FR4 comprising the amino acid sequence of SEQ ID NO:16.

In one aspect, provided herein is an anti-TF antibody comprising a heavy chain variable domain comprising the amino acid sequence of SEQ ID NO:7 or comprising a light chain variable domain comprising the amino acid sequence of SEQ ID NO:8. In one aspect, provided herein is an anti-TF antibody comprising a heavy chain variable domain comprising the amino acid sequence of SEQ ID NO:7 and comprising a light chain variable domain comprising the amino acid sequence of SEQ ID NO:8.

In some embodiments, provided herein is an anti-TF antibody comprising a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:7. In certain embodiments, a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:7 contains substitutions (e.g., conservative substitutions), insertions, or deletions relative to the reference sequence and retains the ability to bind to a TF (e.g., human TF). In certain embodiments, a total of 1 to 10 amino acids have been substituted, inserted and/or deleted in SEQ ID NO:7. In certain embodiments, substitutions, insertions, or deletions (e.g., 1, 2, 3, 4, or 5 amino acids) occur in regions outside the CDRs (i.e., in the FRs). In some embodiments, the anti-TF antibody comprises a heavy chain variable domain sequence of SEQ ID NO:7 including post-translational modifications of that sequence. In a particular embodiment, the heavy chain variable domain comprises one, two or three CDRs selected from: (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3.

In some embodiments, provided herein is an anti-TF antibody comprising a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:8. In certain embodiments, a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:8 contains substitutions (e.g., conservative substitutions), insertions, or deletions relative to the reference sequence and retains the ability to bind to a TF (e.g., human TF). In certain embodiments, a total of 1 to 10 amino acids have been substituted, inserted and/or deleted in SEQ ID NO:8. In certain embodiments, substitutions, insertions, or deletions (e.g., 1, 2, 3, 4, or 5 amino acids) occur in regions outside the CDRs (i.e., in the FRs). In some embodiments, the anti-TF antibody comprises a light chain variable domain sequence of SEQ ID NO:8 including post-translational modifications of that sequence. In a particular embodiment, the light chain variable domain comprises one, two or three CDRs selected from: (a) CDR-L1 comprising the amino acid sequence of SEQ ID NO:4, (b) CDR-L2 comprising the amino acid sequence of SEQ ID NO:5, and (c) CDR-L3 comprising the amino acid sequence of SEQ ID NO:6.

In some embodiments, the anti-TF antibody comprises a heavy chain variable domain as in any of the embodiments provided above, and a light chain variable domain as in any of the embodiments provided above. In one embodiment, the antibody comprises the heavy chain variable domain sequence of SEQ ID NO:7 and the light chain variable domain sequence of SEQ ID NO:8, including post-translational modifications of those sequences.

In some embodiments, the anti-TF antibody of the anti-TF antibody-drug conjugate comprises: i) a heavy chain CDR1 comprising the amino acid sequence of SEQ ID NO: 1, a heavy chain CDR2 comprising the amino acid sequence of SEQ ID NO: 2, a heavy chain CDR3 comprising the amino acid sequence of SEQ ID NO: 3; and ii) a light chain CDR1 comprising the amino acid sequence of SEQ ID NO: 4, a light chain CDR2 comprising the amino acid sequence of SEQ ID NO: 5, and a light chain CDR3 comprising the amino acid sequence of SEQ ID NO: 6.

In some embodiments, the anti-TF antibody of the anti-TF antibody-drug conjugate comprises: i) an amino acid sequence having at least 85% sequence identity to a heavy chain variable region comprising the amino acid sequence of SEQ ID NO: 7, and ii) an amino acid sequence having at least 85% sequence identity to a light chain variable region comprising the amino acid sequence of SEQ ID NO: 8.

In some embodiments, the anti-TF antibody of the anti-TF antibody-drug conjugate is a monoclonal antibody.

In some embodiments, the anti-TF antibody of the anti-TF antibody-drug conjugate is tisotumab, which is also known as antibody 011 as described in WO 2011/157741 and WO 2010/066803.

Anti-TF antibodies of the present invention may also be described or specified in terms of their binding affinity to TF (e.g., human TF). Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

There are five classes of immunoglobulins: IgA, IgD, IgE, IgG and IgM, having heavy chains designated α, δ, ε, γ and μ, respectively. The γ and α classes are further divided into subclasses e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1.

The antibodies also include derivatives that are modified, i.e., by the covalent attachment of any type of molecule to the antibody such that covalent attachment does not prevent the antibody from binding to TF or from exerting a cytostatic or cytotoxic effect on HD cells. For example, but not by way of limitation, the antibody derivatives include antibodies that have been modified, e.g., by glycosylation, acetylation, PEGylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

### B. Antibody-Drug Conjugate Structure

In some aspects, the anti-TF antibody-drug conjugates described herein comprise a linker between an anti-TF antibody or antigen-binding fragment thereof as described herein and a cytostatic or cytotoxic drug. In some embodiments the linker is a non-cleavable linker. In some embodiments the linker is a cleavable linker.

In some embodiments, the linker is a cleavable peptide linker comprising maleimido caproyl (MC), the dipeptide valine-citrulline (vc) and p-aminobenzylcarbamate (PAB). In some embodiments, the cleavable peptide linker has the formula: MC-vc-PAB-, wherein:
a) MC is:

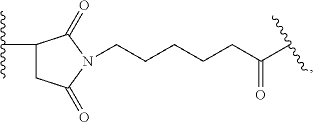 [SMILES: *C(=O)CCCCCN1C(=O)CC(*)C1=O]

b) vc is the dipeptide valine-citrulline, and
c) PAB is:

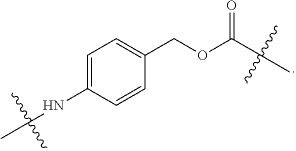 [SMILES: *Nc1ccc(COC(*)=O)cc1]

In some embodiments, the linker is a cleavable peptide linker comprising maleimido caproyl (MC). In some embodiments, the cleavable peptide linker has the formula: MC-, wherein:
a) MC is:

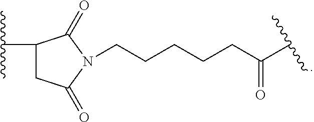 [SMILES: *C(=O)CCCCCN1C(=O)CC(*)C1=O]

In some embodiments, the linker is attached to sulphydryl residues of the anti-TF antibody or antigen-binding fragment thereof obtained by partial or full reduction of the anti-TF antibody or antigen-binding fragment thereof. In some embodiments, the linker is attached to sulphydryl residues of the anti-TF antibody or antigen-binding fragment thereof obtained by partial reduction of the anti-TF antibody or antigen-binding fragment thereof. In some embodiments, the linker is attached to sulphydryl residues of the anti-TF antibody or antigen-binding fragment thereof obtained by full reduction of the anti-TF antibody or antigen-binding fragment thereof.

In some aspects, the anti-TF antibody-drug conjugates described herein comprise a linker as described herein between an anti-TF antibody or antigen-binding fragment thereof as described herein and a cytostatic or cytotoxic drug. Auristatins have been shown to interfere with microtubule dynamics, GTP hydrolysis and nuclear and cellular division (See Woyke et al (2001) Antimicrob. Agents and Chemother. 45(12): 3580-3584) and have anti-cancer (See U.S. Pat. No. 5,663,149) and antifungal activity (See Pettit et al., (1998) Antimicrob. Agents and Chemother. 42: 2961-2965. For example, auristatin E can be reacted with para-acetyl benzoic acid or benzoylvaleric acid to produce AEB and AEVB, respectively. Other typical auristatin derivatives include AFP, MMAF (monomethyl auristatin F), and MMAE (monomethyl auristatin E). Suitable auristatins and auristatin analogs, derivatives and prodrugs, as well as suitable linkers for conjugation of auristatins to Abs, are described in, e.g., U.S. Pat. Nos. 5,635,483, 5,780,588 and 6,214,345 and in International patent application publications WO02088172, WO2004010957, WO2005081711, WO2005084390, WO2006132670, WO03026577, WO200700860, WO207011968 and WO205082023. In some embodiments of the anti-TF antibody-drug conjugates described herein, the cytostatic or cytotoxic drug is an auristatin or a functional analog thereof (e.g., functional peptide thereof) or a functional derivative thereof. In some embodiments, the auristatin is a monomethyl auristatin or a functional analog thereof (e.g., functional peptide thereof) or a functional derivative thereof.

In one embodiment, the auristatin is monomethyl auristatin E (MMAE):

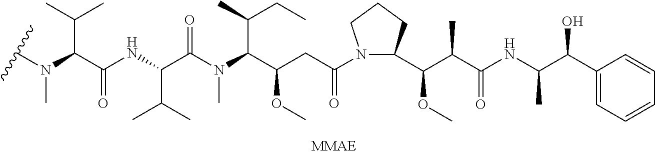 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

wherein the wavy line indicates the attachment site for the linker.

In one embodiment, the auristatin is monomethyl auristatin F (MMAF):

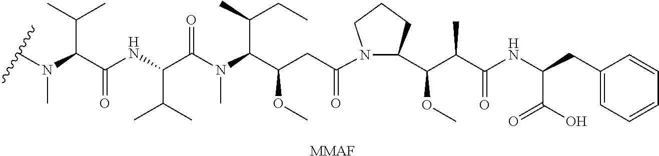

wherein the wavy line indicates the attachment site for the linker.

In one embodiment, the cleavable peptide linker has the formula: MC-vc-PAB-, and is attached to MMAE. The resulting linker-auristatin, MC-vc-PAB-MMAE is also designated vcMMAE. The vcMMAE drug linker moiety and conjugation methods are disclosed in WO2004010957, U.S. Pat. Nos. 7,659,241, 7,829,531 and 7,851,437. When vcMMAE is attached to an anti-TF antibody or antigen-binding fragment thereof as described herein, the resulting structure is:

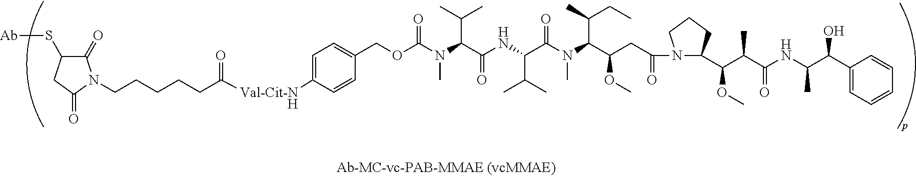

wherein p denotes a number from 1 to 8, e.g., 1, 2, 3, 4, 5, 6, 7 or 8, e.g., p may be from 3-5, S represents a sulphydryl residue of the anti-TF antibody and Ab designates an anti-TF antibody or antigen-binding fragment thereof as described herein. In one embodiment, the average value of p in a population of antibody-drug conjugates is about 4. In some embodiments, p is measured by hydrophobic interaction chromatography (HIC), for example by resolving drug-loaded species based on the increasing hydrophobicity with the least hydrophobic, unconjugated form eluting first and the most hydrophobic, 8-drug form eluting last with the area percentage of a peak representing the relative distribution of the particular drug-loaded antibody-drug conjugate species. See Ouyang, J., 2013, Antibody-Drug Conjugates, Methods in Molecular Biology (Methods and Protocols). In some embodiments, p is measured by reversed phase high-performance liquid chromatography (RP-HPLC), for example by first performing a reduction reaction to completely dissociate the heavy and light chains of the ADC, then separating the light and heavy chains and their corresponding drug-loaded forms on an RP column, where the percentage peak are from integration of the light chain and heavy chain peaks, combined with the assigned drug load for each peak, is used to calculate the weighted average drug to antibody ration. See Ouyang, J., 2013, Antibody-Drug Conjugates, Methods in Molecular Biology (Methods and Protocols).

In one embodiment, the cleavable peptide linker has the formula: MC-vc-PAB-, and is attached to MMAF. The resulting linker-auristatin, MC-vc-PAB-MMAF is also designated vcMMAF. The vcMMAF drug linker moiety and conjugation methods are disclosed in WO2005081711 and U.S. Pat. No. 7,498,298. When vcMMAF is attached to an anti-TF antibody or antigen-binding fragment thereof as described herein, the resulting structure is:

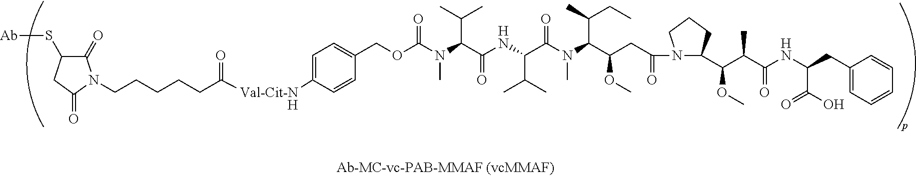

wherein p denotes a number from 1 to 8, e.g., 1, 2, 3, 4, 5, 6, 7 or 8, e.g., p may be from 3-5, S represents a sulphydryl residue of the anti-TF antibody and Ab designates an anti-TF antibody or antigen-binding fragment thereof as described herein. In one embodiment, the average value of p in a population of antibody-drug conjugates is about 4. In some embodiments, p is measured by hydrophobic interaction chromatography (HIC), for example by resolving drug-loaded species based on the increasing hydrophobicity with the least hydrophobic, unconjugated form eluting first and the most hydrophobic, 8-drug form eluting last with the area percentage of a peak representing the relative distribution of the particular drug-loaded antibody-drug conjugate species. See Ouyang, J., 2013, Antibody-Drug Conjugates, Methods in Molecular Biology (Methods and Protocols). In some embodiments, p is measured by reversed phase high-performance liquid chromatography (RP-HPLC), for example by first performing a reduction reaction to completely dissociate the heavy and light chains of the ADC, then separating the light and heavy chains and their corresponding drug-loaded forms on an RP column, where the percentage peak are from integration of the light chain and heavy chain peaks, combined with the assigned drug load for each peak, is used to calculate the weighted average drug to antibody ration. See Ouyang, J., 2013, Antibody-Drug Conjugates, Methods in Molecular Biology (Methods and Protocols).

In one embodiment, the antibody-drug conjugate is tisotumab vedotin.

### C. Anti-PD-1 Antibody

Generally, anti-PD-1 antibodies or antigen-binding fragments thereof of the disclosure bind to PD-1, e.g., human PD-1. In some embodiments, the anti-PD-1 antibody or antigen-binding fragment thereof comprises the complementary determining regions (CDRs) of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof comprises the CDRs of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof is nivolumab. See, e.g., U.S. Pat. No. 8,008,449; WO 2013/173223; WO 2006/121168. The antibody nivolumab is also known as OPDIVO®. In some embodiments, the anti-PD-1 antibody or antigen-binding fragment thereof is Amp-514. See, e.g., Naing et al., Annals of Oncology, Volume 27, Issue suppl_6, 1 Oct. 2016, 1072P. The antibody Amp-514 is also known as MEDI0680. In some embodiments, the anti-PD-1 antibody or antigen-binding fragment thereof is tislelizumab. See, e.g., U.S. Pat. No. 9,834,606. The antibody tislelizumab is also known as BGB-A317. In some embodiments, the anti-PD-1 antibody or antigen-binding fragment thereof is cemiplimab. See, e.g., Burova et al., Mol Cancer Ther. 2017 May; 16(5):861-870. The antibody cemiplimab is also known as REGN2810. In some embodiments, the anti-PD-1 antibody or antigen-binding fragment thereof is TSR-042 (readily available on the world wide web at www.ejcancer.com/article/S0959-8049(16) 32902-1/pdf). In some embodiments, the anti-PD-1 antibody or antigen-binding fragment thereof is JNJ-63723283. See, e.g., Calvo et al., Journal of Clinical Oncology 36, no. 5_suppl (February 2018) 58-58. The antibody JNJ-63723283 is also known as JNJ-3283. In some embodiments, the anti-PD-1 antibody or antigen-binding fragment thereof is CBT-501. See, e.g., Patel et al., Journalfor ImmunoTherapy of Cancer, 2017, 5 (Suppl 2):P242. The antibody CBT-501 is also known as genolimzumab. In some embodiments, the anti-PD-1 antibody or antigen-binding fragment thereof is PF-06801591. See, e.g., Youssef et al., Proceedings of the American Associationfor Cancer Research Annual Meeting 2017; Cancer Res 2017; 77 (13 Suppl):Abstract. In some embodiments, the anti-PD-1 antibody or antigen-binding fragment thereof is JS-001. See, e.g., US 2016/0272708. In some embodiments, the anti-PD-1 antibody or antigen-binding fragment thereof is camrelizumab. See, e.g., U.S. Patent Publication US2016/376367; Huang et al., Clinical Cancer Research 2018 Mar. 15; 24(6):1296-1304. The antibody camrelizumab is also known as SHR-1210 and INCSHR-1210. In some embodiments, the anti-PD-1 antibody or antigen-binding fragment thereof is PDR001. See, e.g., WO2017/106656; Naing et al., Journal of Clinical Oncology 34, no. 15_suppl (May 2016) 3060-3060. In some embodiments, the anti-PD-1 antibody or antigen-binding fragment thereof is BCD-100. See, e.g., WO2018/103017. In some embodiments, the anti-PD-1 antibody or antigen-binding fragment thereof is AGEN2034. See, e.g., WO2017/040790. In some embodiments, the anti-PD-1 antibody or antigen-binding fragment thereof is IBI-308. See, e.g., WO2017/024465; WO2017/133540. In some embodiments, the anti-PD-1 antibody or antigen-binding fragment thereof is BI-754091. See, e.g., U.S. Patent Publication US2017/334995; Johnson et al., Journal of Clinical Oncology 36, no. 5_suppl (February 2018) 212-212. In some embodiments, the anti-PD-1 antibody or antigen-binding fragment thereof is GLS-010. See, e.g., WO2017/025051. In some embodiments, the anti-PD-1 antibody or antigen-binding fragment thereof is LZM-009. See, e.g., U.S. Patent Publication US2017/210806. In some embodiments, the anti-PD-1 antibody or antigen-binding fragment thereof is AK-103. See, e.g., WO2017/071625; WO2017/166804; WO2018/036472. In some embodiments, the anti-PD-1 antibody or antigen-binding fragment thereof is MGA-012. See, e.g., WO2017/019846. In some embodiments, the anti-PD-1 antibody or antigen-binding fragment thereof is Sym-021. See, e.g., WO2017/055547. In some embodiments, the anti-PD-1 antibody or antigen-binding fragment thereof is CS1003. See, e.g., CN107840887. In some embodiments, the anti-PD-1 antibody or antigen-binding fragment thereof comprises a heavy chain variable region and a light chain variable region, wherein the heavy chain variable region comprises:
(i) a CDR-H1 comprising the amino acid sequence of SEQ ID NO:17;
(ii) a CDR-H2 comprising the amino acid sequence of SEQ ID NO:18; and
(iii) a CDR-H3 comprising the amino acid sequence of SEQ ID NO:19; and wherein the light chain variable region comprises:
(i) a CDR-L1 comprising the amino acid sequence of SEQ ID NO:20;
(ii) a CDR-L2 comprising the amino acid sequence of SEQ ID NO:21; and
(iii) a CDR-L3 comprising the amino acid sequence of SEQ ID NO:22. In some embodiments, the CDRs of the anti-PD-1 antibody are delineated using the Kabat numbering scheme (Kabat, E. A., et al. (1991) Sequences of Proteins of Immunological Interest, Fifth Edition, U.S. Department of Health and Human Services, NTH Publication No. 91-3242).

Anti-PD-1 antibodies of the disclosure are preferably monoclonal, and may be multispecific, human, humanized or chimeric antibodies, single chain antibodies, Fab fragments, F(ab′) fragments, fragments produced by a Fab expression library, and PD-1 binding fragments of any of the above. In some embodiments, an anti-PD-1 antibody described herein binds specifically to PD-1 (e.g., human PD-1). The immunoglobulin molecules of the disclosure can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the disclosure, the antibodies are antigen-binding fragments (e.g., human antigen-binding fragments) as described herein and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding fragments, including single-chain antibodies, may comprise the variable region(s) alone or in combination with the entirety or a portion of the following: hinge region, CH1, CH2, CH3 and CL domains. Also included in the present disclosure are antigen-binding fragments comprising any combination of variable region(s) with a hinge region, CH1, CH2, CH3 and CL domains. In some embodiments, the anti-PD-1 antibodies or antigen-binding fragments thereof are human, murine (e.g., mouse and rat), donkey, sheep, rabbit, goat, guinea pig, camelid, horse, or chicken.

The anti-PD-1 antibodies of the present disclosure may be monospecific, bispecific, trispecific or of greater multi specificity. Multispecific antibodies may be specific for different epitopes of PD-1 or may be specific for both PD-1 as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; Kostelny et al., 1992, J. Immunol. 148:1547 1553.

Anti-PD-1 antibodies of the present disclosure may be described or specified in terms of the particular CDRs they comprise. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme). The boundaries of a given CDR may vary depending on the scheme used for identification. In some embodiments, a “CDR” or “complementarity determining region,” or individual specified CDRs (e.g., CDR-H1, CDR-H2, CDR-H3), of a given antibody or region thereof (e.g., variable region thereof) should be understood to encompass a (or the specific) CDR as defined by any of the aforementioned schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes. The scheme for identification of a particular CDR or CDRs may be specified, such as the CDR as defined by the Kabat, Chothia, AbM or IMGT method.

In some embodiments, numbering of amino acid residues in CDR sequences of anti-PD-1 antibodies or antigen-binding fragments thereof provided herein are according to the IMGT numbering scheme as described in Lefranc, M. P. et al., Dev. Comp. Immunol., 2003, 27, 55-77.

In some embodiments, the anti-PD-1 antibodies of the present disclosure comprise the CDRs of the antibody nivolumab. See WO 2006/121168. In some embodiments, the CDRs of the antibody nivolumab are delineated using the Kabat numbering scheme (Kabat, E. A, et al (1991) Sequences of Proteins of Immunological Interest, Fifth Edition, U.S. Department of Health and Human Services, NTH Publication No. 91-3242). The present disclosure encompasses an anti-PD-1 antibody or derivative thereof comprising a heavy or light chain variable domain, said variable domain comprising (a) a set of three CDRs, in which said set of CDRs are from the monoclonal antibody nivolumab, and (b) a set of four framework regions, in which said set of framework regions differs from the set of framework regions in the monoclonal antibody nivolumab, and in which said anti-PD-1 antibody or derivative thereof binds to PD-1. In certain embodiments, the anti-PD-1 antibody is nivolumab. The antibody nivolumab is also known as OPDIVO®.

In one aspect, provided herein is an anti-PD-1 antibody comprising a heavy chain variable region and a light chain variable region, wherein the heavy chain variable region comprises (i) CDR-H1 comprising the amino acid sequence of SEQ ID NO:17, (ii) CDR-H2 comprising the amino acid sequence of SEQ ID NO:18, and (iii) CDR-H3 comprising the amino acid sequence of SEQ ID NO:19; and wherein the light chain variable region comprises (i) CDR-L1 comprising the amino acid sequence of SEQ ID NO:20, (ii) CDR-L2 comprising the amino acid sequence of SEQ ID NO:21, and (iii) CDR-L3 comprising the amino acid sequence of SEQ ID NO:22. In some embodiments, the CDRs of the anti-PD-1 antibody, are delineated using the Kabat numbering scheme.

In one embodiment, an anti-PD-1 antibody comprises a light chain variable domain comprising a framework sequence and hypervariable regions, wherein the framework sequence comprises the LC-FR1-LC-FR4 amino acid sequences of SEQ ID NO:27 (LC-FR1), SEQ ID NO:28 (LC-FR2), SEQ ID NO:29 (LC-FR3), and SEQ ID NO:30 (LC-FR4), respectively; the CDR-L1 comprises the amino acid sequence of SEQ ID NO:20; the CDR-L2 comprises the amino acid sequence of SEQ ID NO:21; and the CDR-L3 comprises the amino acid sequence of SEQ ID NO:22. In some embodiments, the CDRs of the anti-PD-1 antibody are delineated using the Kabat numbering scheme.

In some embodiments of the anti-PD-1 antibodies described herein, the heavy chain variable domain comprises the amino acid sequence of QVQLVESGGGVVQPGRSLRLDCKASGITFSNSGMHWVRQAPGKGLEWVAVIWYDG SKRYYADSVKGRFTISRDNSKNTLFLQMNSLRAEDTAVYYCATNDDYWGQGTLVTV SS (SEQ ID NO:31) and the light chain variable domain comprises the amino acid sequence of

(SEQ ID NO: 32)
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIK.

In some embodiments of the anti-PD-1 antibodies described herein, the heavy chain CDR sequences comprise the following:
a) CDR-H1 (NSGMH (SEQ ID NO:17));
b) CDR-H2 (VIWYDGSKRYYADSVKG (SEQ ID NO:18)); and
c) CDR-H3 (NDDY (SEQ ID NO:19)). In some embodiments, the CDRs of the anti-PD-1 antibody are delineated using the Kabat numbering scheme.

In some embodiments of the anti-PD-1 antibodies described herein, the heavy chain FR sequences comprise the following:

a) HC-FR1
(QVQLVESGGGVVQPGRSLRLDCKASGITFS (SEQ ID NO: 23));
b) HC-FR2
(WVRQAPGKGLEWVA (SEQ ID NO: 24));
c) HC-FR3
(RFTISRDNSKNTLFLQMNSLRAEDTAVYYCAT (SEQ ID NO:
25));
and
d) HC-FR4
(WGQGTLVTVSS (SEQ ID NO: 26)).

In some embodiments of the anti-PD-1 antibodies described herein, the light chain CDR sequences comprise the following:
a) CDR-L1 (RASQSVSSYLA (SEQ ID NO:20));
b) CDR-L2 (DASNRAT (SEQ ID NO:21)); and
c) CDR-L3 (QQSSNWPRT (SEQ ID NO:22)). In some embodiments, the CDRs of the anti-PD-1 antibody are delineated using the Kabat numbering scheme.

In some embodiments of the anti-PD-1 antibodies described herein, the light chain FR sequences comprise the following:
a) LC-FR1 (EIVLTQSPATLSLSPGERATLSC (SEQ ID NO:27));
b) LC-FR2 (WYQQKPGQAPRLLIY (SEQ ID NO:28));

a) LC-FR1
(EIVLTQSPATLSLSPGERATLSC (SEQ ID NO: 27));
b) LC-FR2
(WYQQKPGQAPRLLIY (SEQ ID NO: 28));
c) LC-FR3
(GIPARFSGSGSGTDFTLTISSLEPEDFAVYYC (SEQ ID
NO: 29));
and
d) LC-FR4
(FGQGTKVEIK (SEQ ID NO: 30)).

In some embodiments, provided herein is an anti-PD-1 antibody that binds to PD-1 (e.g., human PD-1), wherein the antibody comprises a heavy chain variable region and a light chain variable region, wherein the antibody comprises:
(a) heavy chain variable domain comprising:
   (1) an HC-FR1 comprising the amino acid sequence of SEQ ID NO:23;
   (2) an CDR-H1 comprising the amino acid sequence of SEQ ID NO:17;
   (3) an HC-FR2 comprising the amino acid sequence of SEQ ID NO:24;
   (4) an CDR-H2 comprising the amino acid sequence of SEQ ID NO:18;
   (5) an HC-FR3 comprising the amino acid sequence of SEQ ID NO:25;
   (6) an CDR-H3 comprising the amino acid sequence of SEQ ID NO:19; and
   (7) an HC-FR4 comprising the amino acid sequence of SEQ ID NO:26, and/or
(b) a light chain variable domain comprising:
   (1) an LC-FR1 comprising the amino acid sequence of SEQ ID NO:27;
   (2) an CDR-L1 comprising the amino acid sequence of SEQ ID NO:20;
   (3) an LC-FR2 comprising the amino acid sequence of SEQ ID NO:28;
   (4) an CDR-L2 comprising the amino acid sequence of SEQ ID NO:21;
   (5) an LC-FR3 comprising the amino acid sequence of SEQ ID NO:29;
   (6) an CDR-L3 comprising the amino acid sequence of SEQ ID NO:22; and
   (7) an LC-FR4 comprising the amino acid sequence of SEQ ID NO:30. In some embodiments, the CDRs of the anti-PD-1 antibody are delineated using the Kabat numbering scheme.

In one aspect, provided herein is an anti-PD-1 antibody comprising a heavy chain variable domain comprising the amino acid sequence of SEQ ID NO:31 or comprising a light chain variable domain comprising the amino acid sequence of SEQ ID NO:32. In one aspect, provided herein is an anti-PD-1 antibody comprising a heavy chain variable domain comprising the amino acid sequence of SEQ ID NO:31 and comprising a light chain variable domain comprising the amino acid sequence of SEQ ID NO:32.

In some embodiments, provided herein is an anti-PD-1 antibody comprising a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:31. In certain embodiments, a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:31 contains substitutions (e.g., conservative substitutions), insertions, or deletions relative to the reference sequence and retains the ability to bind to a PD-1 (e.g., human PD-1). In certain embodiments, a total of 1 to 10 amino acids have been substituted, inserted and/or deleted in SEQ ID NO:31. In certain embodiments, substitutions, insertions, or deletions (e.g., 1, 2, 3, 4, or 5 amino acids) occur in regions outside the CDRs (i.e., in the FRs). In some embodiments, the anti-PD-1 antibody comprises a heavy chain variable domain sequence of SEQ ID NO:31 including post-translational modifications of that sequence. In a particular embodiment, the heavy chain variable domain comprises one, two or three CDRs selected from: (a) CDR-H1 comprising the amino acid sequence of SEQ ID NO:17, (b) CDR-H2 comprising the amino acid sequence of SEQ ID NO:18, and (c) CDR-H3 comprising the amino acid sequence of SEQ ID NO: 19. In some embodiments, the CDRs of the anti-PD-1 antibody are delineated using the Kabat numbering scheme.

In some embodiments, provided herein is an anti-PD-1 antibody comprising a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:32. In certain embodiments, a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:32 contains substitutions (e.g., conservative substitutions), insertions, or deletions relative to the reference sequence and retains the ability to bind to a PD-1 (e.g., human PD-1). In certain embodiments, a total of 1 to 10 amino acids have been substituted, inserted and/or deleted in SEQ ID NO:32. In certain embodiments, substitutions, insertions, or deletions (e.g., 1, 2, 3, 4, or 5 amino acids) occur in regions outside the CDRs (i.e., in the FRs). In some embodiments, the anti-PD-1 antibody comprises a light chain variable domain sequence of SEQ ID NO:32 including post-translational modifications of that sequence. In a particular embodiment, the light chain variable domain comprises one, two or three CDRs selected from: (a) CDR-L1 comprising the amino acid sequence of SEQ ID NO:20, (b) CDR-L2 comprising the amino acid sequence of SEQ ID NO:21, and (c) CDR-L3 comprising the amino acid sequence of SEQ ID NO:22. In some embodiments, the CDRs of the anti-PD-1 antibody are delineated using the Kabat numbering scheme.

In some embodiments, the anti-PD-1 antibody comprises a heavy chain variable domain as in any of the embodiments provided above, and a light chain variable domain as in any of the embodiments provided above. In one embodiment, the antibody comprises the heavy chain variable domain sequence of SEQ ID NO:31 and the light chain variable domain sequence of SEQ ID NO:32, including post-translational modifications of those sequences.

In some embodiments, the anti-PD-1 antibody comprises: i) a heavy chain CDR1 comprising the amino acid sequence of SEQ ID NO: 17, a heavy chain CDR2 comprising the amino acid sequence of SEQ ID NO: 18, a heavy chain CDR3 comprising the amino acid sequence of SEQ ID NO: 19; and ii) a light chain CDR1 comprising the amino acid sequence of SEQ ID NO: 20, a light chain CDR2 comprising the amino acid sequence of SEQ ID NO: 21, and a light chain CDR3 comprising the amino acid sequence of SEQ ID NO: 22. In some embodiments, the CDRs of the anti-PD-1 antibody are delineated using the Kabat numbering scheme.

In some embodiments, the anti-PD-1 antibody comprises: i) an amino acid sequence having at least 85% sequence identity to a heavy chain variable region comprising the amino acid sequence of SEQ ID NO: 31, and ii) an amino acid sequence having at least 85% sequence identity to a light chain variable region comprising the amino acid sequence of SEQ ID NO: 32.

In some embodiments, the anti-PD-1 antibody is a monoclonal antibody.

In some embodiments, the anti-PD-1 antibody is nivolumab, which is also known as antibody OPDIVO® as described in WO 2006/121168.

Anti-PD-1 antibodies of the present invention may also be described or specified in terms of their binding affinity to PD-1 (e.g., human PD-1). Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10-s M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

There are five classes of immunoglobulins: IgA, IgD, IgE, IgG and IgM, having heavy chains designated α, δ, ε, γ and μ, respectively. The γ and α classes are further divided into subclasses e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1.

The antibodies also include derivatives that are modified, i.e., by the covalent attachment of any type of molecule to the antibody such that covalent attachment does not prevent the antibody from binding to PD-1. For example, but not by way of limitation, the antibody derivatives include antibodies that have been modified, e.g., by glycosylation, acetylation, PEGylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

### D. Nucleic Acids, Host Cells and Methods of Production

In some aspects, also provided herein are nucleic acids encoding an anti-TF antibody or antigen-binding fragment thereof as described herein or an anti-PD-1 antibody or antigen-binding fragment thereof as described herein. Further provided herein are vectors comprising the nucleic acids encoding an anti-TF antibody or antigen-binding fragment thereof as described herein or an anti-PD-1 antibody or antigen-binding fragment thereof as described herein. Further provided herein are host cells expressing the nucleic acids encoding an anti-TF antibody or antigen-binding fragment thereof as described herein or an anti-PD-1 antibody or antigen-binding fragment thereof as described herein. Further provided herein are host cells comprising the vectors comprising the nucleic acids encoding an anti-TF antibody or antigen-binding fragment thereof as described herein or an anti-PD-1 antibody or antigen-binding fragment thereof as described herein. Methods of producing an anti-TF antibody, linker and anti-TF antibody-drug conjugate are described in U.S. Pat. No. 9,168,314.

The anti-TF antibodies described herein or anti-PD-1 antibodies described herein may be prepared by well-known recombinant techniques using well known expression vector systems and host cells. In one embodiment, the antibodies are prepared in a CHO cell using the GS expression vector system as disclosed in De la Cruz Edmunds et al., 2006, Molecular Biotechnology 34; 179-190, EP216846, U.S. Pat. No. 5,981,216, WO 87/04462, EP323997, U.S. Pat. Nos. 5,591,639, 5,658,759, EP338841, U.S. Pat. Nos. 5,879,936, and 5,891,693.

After isolating and purifying the anti-TF antibodies from the cell media using well known techniques in the art, they are conjugated with an auristatin via a linker as described in U.S. Pat. No. 9,168,314.

Monoclonal anti-TF antibodies described herein or anti-PD-1 antibodies described herein may e.g. be produced by the hybridoma method first described by Kohler et al., Nature, 256, 495 (1975), or may be produced by recombinant DNA methods. Monoclonal antibodies may also be isolated from phage antibody libraries using the techniques described in, for example, Clackson et al., Nature, 352, 624-628 (1991) and Marks et al., JMol, Biol., 222(3):581-597 (1991). Monoclonal antibodies may be obtained from any suitable source. Thus, for example, monoclonal antibodies may be obtained from hybridomas prepared from murine splenic B cells obtained from mice immunized with an antigen of interest, for instance in form of cells expressing the antigen on the surface, or a nucleic acid encoding an antigen of interest. Monoclonal antibodies may also be obtained from hybridomas derived from antibody-expressing cells of immunized humans or non-human mammals such as rats, dogs, primates, etc.

In one embodiment, the antibody (e.g., anti-TF antibody or anti-PD-1 antibody) of the invention is a human antibody. Human monoclonal antibodies directed against TF or PD-1 may be generated using transgenic or transchromosomal mice carrying parts of the human immune system rather than the mouse system. Such transgenic and transchromosomic mice include mice referred to herein as HuMAb mice and KM mice, respectively, and are collectively referred to herein as “transgenic mice”.

The HuMAb mouse contains a human immunoglobulin gene minilocus that encodes unrearranged human heavy (μ and γ) and κ light chain immunoglobulin sequences, together with targeted mutations that inactivate the endogenous μ and κ chain loci (Lonberg, N. et al., Nature, 368, 856-859 (1994)). Accordingly, the mice exhibit reduced expression of mouse IgM or κ and in response to immunization, the introduced human heavy and light chain transgenes undergo class switching and somatic mutation to generate high affinity human IgG,κ monoclonal antibodies (Lonberg, N. et al. (1994), supra; reviewed in Lonberg, N. Handbook of Experimental Pharmacology 113, 49-101 (1994), Lonberg, N. and Huszar. D., Intern. Rev. Immunol, Vol. 13 65-93 (1995) and Harding, F. and Lonberg, N. Ann, N.Y. Acad. Sci 764:536-546 (1995)). The preparation of HuMAb mice is described in detail in Taylor, L. et al., Nucleic Acids Research. 20:6287-6295 (1992), Chen, J. et al., International Immunology. 5:647-656 (1993), Tuaillon at al., J. Immunol, 152:2912-2920 (1994), Taylor, L. et al., International Immunology, 6:579-591 (1994), Fishwild, D. et al., Nature Biotechnology, 14:845-851 (1996). See also U.S. Pat. Nos. 5,545,806, 5,569,825, 5,625,126, 5,633,425, 5,789,650, 5,877,397, 5,661,016, 5,814,318, 5,874,299, 5,770,429, 5,545,807, WO 98/24884, WO 94/25585, WO 93/1227, WO 92/22645, WO 92/03918 and WO 01/09187.

The HCo7 mice have a JKD disruption in their endogenous light chain (kappa) genes (as described in Chen et al, EMBO J. 12:821-830 (1993)), a CMD disruption in their endogenous heavy chain genes (as described in Example 1 of WO 01/14424), a KCo5 human kappa light chain transgene (as described in Fishwild et al., Nature Biotechnology, 14:845-851 (1996)), and a HCo7 human heavy chain transgene (as described in U.S. Pat. No. 5,770,429).

The HCo12 mice have a JKD disruption in their endogenous light chain (kappa) genes (as described in Chen et al., EMBO J. 12:821-830 (1993)), a CMD disruption in their endogenous heavy chain genes (as described in Example 1 of WO 01/14424), a KCo5 human kappa light chain transgene (as described in Fishwild et al., Nature Biotechnology, 14:845-851 (1996)), and a HCo12 human heavy chain transgene (as described in Example 2 of WO 01/14424).

The HCo17 transgenic mouse strain (see also US 2010/0077497) was generated by coinjection of the 80 kb insert of pHC2 (Taylor et al. (1994) Int. Immunol., 6:579-591), the Kb insert of pVX6, and a −460 kb yeast artificial chromosome fragment of the yIgH24 chromosome. This line was designated (HCo17) 25950. The (HCo17) 25950 line was then bred with mice comprising the CMD mutation (described in Example 1 of PCT Publication WO 01109187), the JKD mutation (Chen et al, (1993) EMBO J. 12:811-820), and the (KC05) 9272 transgene (Fishwild et al. (1996) Nature Biotechnology, 14:845-851). The resulting mice express human immunoglobulin heavy and kappa light chain trans genes in a background homozygous for disruption of the endogenous mouse heavy and kappa light chain loci.

The HCo20 transgenic mouse strain is the result of a co-injection of minilocus 30 heavy chain transgene pHC2, the germline variable region (Vh)-containing YAC yIgH10, and the minilocus construct pVx6 (described in WO09097006). The (HCo20) line was then bred with mice comprising the CMD mutation (described in Example 1 of PCT Publication WO 01/09187), the JKD mutation (Chen et al. (1993) EMBO J. 12:811-820), and the (KC05) 9272 trans gene (Fishwild eta). (1996) Nature Biotechnology, 14:845-851). The resulting mice express human 10 immunoglobulin heavy and kappa light chain transgenes in a background homozygous for disruption of the endogenous mouse heavy and kappa light chain loci.

In order to generate HuMab mice with the salutary effects of the Balb/c strain, HuMab mice were crossed with KCO005 [MIK](Balb) mice which were generated by backcrossing the KC05 strain (as described in Fishwild et (1996) Nature Biotechnology, 14:845-851) to wild-type Balb/c mice to generate mice as described in WO09097006. Using this crossing Balb/c hybrids were created for HCol2, HCol7, and HCo20 strains.

In the KM mouse strain, the endogenous mouse kappa light chain gene has been homozygously disrupted as described in Chen et al., EMBO J. 12:811-820 (1993) and the endogenous mouse heavy chain gene has been homozygously disrupted as described in Example 1 of WO 01/09187, This mouse strain carries a human kappa light chain transgene, KCo5, as described in Fishwild et al., Nature Biotechnology, 14:845-851 (1996). This mouse strain also carries a human heavy chain transchromosome composed of chromosome 14 fragment hCF (SC20) as described in WO 02/43478.

Splenocytes from these transgenic mice may be used to generate hybridomas that secrete human monoclonal antibodies according to well-known techniques, Human monoclonal or polyclonal antibodies of the present invention, or antibodies of the present invention originating from other species may also be generated transgenically through the generation of another non-human mammal or plant that is transgenic for the immunoglobulin heavy and light chain sequences of interest and production of the antibody in a recoverable form therefrom. In connection with the transgenic production in mammals, antibodies may be produced in, and recovered from, the milk of goats, cows, or other mammals. See for instance U.S. Pat. Nos. 5,827,690, 5,756,687, 5,750,172 and 5,741,957.

Further, human antibodies of the present invention or antibodies of the present invention from other species may be generated through display-type technologies, including, without limitation, phage display, retroviral display, ribosomal display, and other techniques, using techniques well known in the art and the resulting molecules may be subjected to additional maturation, such as affinity maturation, as such techniques are well known in the art (See for instance Hoogenboom et al., J. Mol, Biol. 227(2):381-388 (1992) (phage display), Vaughan et al., Nature Biotech, 14:309 (1996) (phage display), Hanes and Plucthau, PNAS USA 94:4937-4942 (1997) (ribosomal display), Parmley and Smith, Gene, 73:305-318 (1988) (phage display), Scott, TIBS. 17:241-245 (1992), Cwirla et al., PNAS USA, 87:6378-6382 (1990), Russel et al., Nucl. Acids Research, 21:1081-4085 (1993), Hogenboom et al., Immunol, Reviews, 130:43-68 (1992), Chiswell and McCafferty, TIBTECH, 10:80-84 (1992), and U.S. Pat. No. 5,733,743). If display technologies are utilized to produce antibodies that are not human, such antibodies may be humanized.

### III. Binding Assays and Other Assays

In one aspect, an antibody of the invention is tested for its antigen binding activity, for example, by known methods such as Enzyme-Linked Immunosorbant Assay (ELISA), immunoblotting (e.g., Western blotting), flow cytometry (e.g., FACS™) immunohistochemistry, immunofluorescence, etc.

In another aspect, competition assays may be used to identify an antibody that competes with any one of the antibodies described herein for binding to TF (e.g., tisotumab) or PD-1 (e.g., nivolumab). Cross-competing antibodies can be readily identified based on their ability to cross-compete in standard TF or PD-1 binding assays such as Biacore analysis, ELISA assays or flow cytometry (See, e.g., WO 2013/173223). In certain embodiments, such a competing antibody binds to the same epitope (e.g., a linear or a conformational epitope) that is bound by any one of the antibodies disclosed herein (e.g., tisotumab or nivolumab). Detailed exemplary methods for mapping an epitope to which an antibody binds are provided in Morris “Epitope Mapping Protocols,” in Methods in Molecular Biology Vol. 66 (Humana Press, Totowa, NJ, 1996).

In an exemplary competition assay, immobilized PD-1 is incubated in a solution comprising a first labeled antibody that binds to PD-1 (e.g., nivolumab) and a second unlabeled antibody that is being tested for its ability to compete with the first antibody for binding to PD-1. The second antibody may be present in a hybridoma supernatant. As a control, immobilized PD-1 is incubated in a solution comprising the first labeled antibody but not the second unlabeled antibody. After incubation under conditions permissive for binding of the first antibody to PD-1, excess unbound antibody is removed, and the amount of label associated with immobilized PD-1 is measured. If the amount of label associated with immobilized PD-1 is substantially reduced in the test sample relative to the control sample, then that indicates that the second antibody is competing with the first antibody for binding to PD-1. See, e.g., Harlow et al. Antibodies: A Laboratory Manual. Ch.14 (Cold Spring Harbor Laboratory, Cold Spring Harbor, NY, 1988). In some embodiments, an anti-PD-1 antibody competes for binding to PD-1 with another PD-1 antibody (e.g., nivolumab) if the antibody blocks binding of the other antibody to PD-1 in a competition assay by more than 20%, more than 25%, more than 30%, more than 35%, more than 40%, more than 45%, more than 50%, more than 55%, more than 60%, more than 65%, more than 70%, more than 75%, more than 80%, more than 85%, more than 90%, more than 95%. In some embodiments, an anti-PD-1 antibody does not compete for binding to PD-1 with another PD-1 antibody (e.g., nivolumab) if the antibody blocks binding of the other antibody to PD-1 in a competition assay by less than 20%, less than 15%, less than 10%, less than 9%, less than 8%, less than 7%, less than 6%, less than 5%, less than 4%, less than 3%, less than 2%, less than 1%. In some embodiments, the PD-1 is human PD-1.

Similar competition assays can be performed to determine if an anti-TF antibody competes with tisotumab for binding to TF. In some embodiments, an anti-TF antibody competes for binding to TF with another TF antibody (e.g., tisotumab) if the antibody blocks binding of the other antibody to TF in a competition assay by more than 20%, more than 25%, more than 30%, more than 35%, more than 40%, more than 45%, more than 50%, more than 55%, more than 60%, more than 65%, more than 70%, more than 75%, more than 80%, more than 85%, more than 90%, more than 95%. In some embodiments, an anti-TF antibody does not compete for binding to TF with another TF antibody (e.g., tisotumab) if the antibody blocks binding of the other antibody to TF in a competition assay by less than 20%, less than 15%, less than 10%, less than 9%, less than 8%, less than 7%, less than 6%, less than 5%, less than 4%, less than 3%, less than 2%, less than 1%. In some embodiments, the TF is human TF.

### IV. Methods of Treatment

The invention provides methods for treating cancer in a subject with an anti-TF antibody-drug conjugate described herein and an anti-PD-1 antibody described herein. In one aspect, the antibody-drug conjugate is tisotumab vedotin. In some embodiments, the anti-PD-1 antibody or antigen-binding fragment thereof comprises the complementary determining regions (CDRs) of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof comprises the CDRs of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or an antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or an antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or an antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof comprises a heavy chain variable region and a light chain variable region, wherein the heavy chain variable region comprises:
(i) a CDR-H1 comprising the amino acid sequence of SEQ ID NO:17;
(ii) a CDR-H2 comprising the amino acid sequence of SEQ ID NO:18; and
(iii) a CDR-H3 comprising the amino acid sequence of SEQ ID NO:19; and wherein the light chain variable region comprises:
(i) a CDR-L1 comprising the amino acid sequence of SEQ ID NO:20;
(ii) a CDR-L2 comprising the amino acid sequence of SEQ ID NO:21; and
(iii) a CDR-L3 comprising the amino acid sequence of SEQ ID NO:22. In some embodiments, the CDR_s of the anti-PD-1 antibody are delineated using the Kabat numrbering scheme. In one aspect, the anti-PD-1 antibody is nivolumab. In a particular embodiment, the subject is a human.

In another aspect the present invention provides an antibody-drug conjugate that binds to TF for use in the treatment of cancer wherein the antibody-drug conjugate is for administration, or to be administered in combination with an anti-PD-1 antibody or an antigen-binding fragment thereof wherein the antibody-drug conjugate comprises an anti-TF antibody or an antigen-binding fragment thereof conjugated to a monomethyl auristatin or a functional analog thereof or a functional derivative thereof, wherein the anti-PD-1 antibody or the antigen-binding fragment thereof inhibits PD-1 activity, and wherein the anti-PD-1 antibody or an antigen-binding fragment thereof comprises the complementary determining regions (CDRs) of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof comprises the CDRs of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof comprises a heavy chain variable region and a light chain variable region, wherein the heavy chain variable region comprises:
(i) a CDR-H1 comprising the amino acid sequence of SEQ ID NO:17;
(ii) a CDR-H2 comprising the amino acid sequence of SEQ ID NO:18; and
(iii) a CDR-H3 comprising the amino acid sequence of SEQ ID NO:19; and wherein the light chain variable region comprises:
(i) a CDR-L1 comprising the amino acid sequence of SEQ ID NO:20;
(ii) a CDR-L2 comprising the amino acid sequence of SEQ ID NO:21; and
(iii) a CDR-L3 comprising the amino acid sequence of SEQ ID NO:22. In some embodiments, the CDRs of the anti-PD-1 antibody are delineated using the Kabat numbering scheme. In one aspect, the anti-PD-1 antibody is nivolumab. In a particular embodiment, the subject is a human.

In another aspect the present invention provides an anti-PD-1 antibody or an antigen-binding fragment thereof for use in the treatment of cancer wherein the anti-PD-1 antibody is for administration, or to be administered in combination with an antibody-drug conjugate that binds to TF wherein the antibody-drug conjugate comprises an anti-TF antibody or an antigen-binding fragment thereof conjugated to a monomethyl auristatin or a functional analog thereof or a functional derivative thereof, wherein the anti-PD-1 antibody or the antigen-binding fragment thereof inhibits PD-1 activity, and wherein the anti-PD-1 antibody or the antigen-binding fragment thereof comprises the complementary determining regions (CDRs) of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof comprises the CDRs of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof comprises the heavy chain variable region and the light chain variable region of antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof comprises a heavy chain variable region and a light chain variable region, wherein the heavy chain variable region comprises:
(i) a CDR-H1 comprising the amino acid sequence of SEQ ID NO:17;
(ii) a CDR-H2 comprising the amino acid sequence of SEQ ID NO:18; and
(iii) a CDR-H3 comprising the amino acid sequence of SEQ ID NO:19; and wherein the light chain variable region comprises:
(i) a CDR-L1 comprising the amino acid sequence of SEQ ID NO:20;
(ii) a CDR-L2 comprising the amino acid sequence of SEQ ID NO:21; and
(iii) a CDR-L3 comprising the amino acid sequence of SEQ ID NO:22. In some embodiments, the CDRs of the anti-PD-1 antibody are delineated using the Kabat numbering scheme. In one aspect, the anti-PD-1 antibody is nivolumab. In a particular embodiment, the subject is a human.

### A. Breast Cancer

The 2014 World Cancer Report from WHO (The World health organization) reports that breast cancer is the second most common cancer worldwide, accounting for just over 1 million new cases annually. It states that in 2000 about 400,000 women died from breast cancer, representing 1.6 percent of all female deaths. The proportion of breast cancer deaths was far higher in the rich countries (2 percent of all female deaths) than in economically poor regions (0.5 percent). Thus, breast cancer is strongly related to the Western lifestyle. As developing countries succeed in achieving lifestyles similar to Europe, North America, Australia, New Zealand and Japan, they will also encounter much higher cancer rates, particularly cancers of the breast. Recent data supports this prediction and show a 20% increase in breast cancer from 2008 to 2012. (Carter D. “New global survey shows an increasing cancer burden”. Am J Nurs. 2014 March; 114(3): 17).

In some aspects, the invention provides methods for treating breast cancer in a subject with an anti-TF antibody-drug conjugate described herein and an anti-PD-1 antibody described herein. In one aspect, the antibody-drug conjugate is tisotumab vedotin. In some embodiments, the anti-PD-1 antibody or antigen-binding fragment thereof comprises the complementary determining regions (CDRs) of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof comprises the CDRs of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof comprises a heavy chain variable region and a light chain variable region, wherein the heavy chain variable region comprises:
(i) a CDR-H1 comprising the amino acid sequence of SEQ ID NO:17;
(ii) a CDR-H2 comprising the amino acid sequence of SEQ ID NO:18; and
(iii) a CDR-H3 comprising the amino acid sequence of SEQ ID NO:19; and wherein the light chain variable region comprises:
(i) a CDR-L1 comprising the amino acid sequence of SEQ ID NO:20;
(ii) a CDR-L2 comprising the amino acid sequence of SEQ ID NO:21; and
(iii) a CDR-L3 comprising the amino acid sequence of SEQ ID NO:22. In some embodiments, the CDRs of the anti-PD-1 antibody are delineated using the Kabat numbering scheme. In one aspect, the anti-PD-1 antibody is nivolumab. In a particular embodiment, the subject is a human.

In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the breast cancer cells from the subject express TF. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of the breast cancer cells from the subject express TF. In some embodiments, the percentage of cells that express TF is determined using immunohistochemistry (IHC). In some embodiments, the percentage of cells that express TF is determined using flow cytometry. In some embodiments, the percentage of cells that express TF is determined using an enzyme-linked immunosorbent assay (ELISA).

In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the breast cancer cells from the subject express PD-L1. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of the breast cancer cells from the subject express PD-L1. In some embodiments, the percentage of cells that express PD-L1 is determined using immunohistochemistry (IHC). In some embodiments, the percentage of cells that express PD-L1 is determined using flow cytometry. In some embodiments, the percentage of cells that express PD-L1 is determined using an enzyme-linked immunosorbent assay (ELISA).

In some embodiments, a tumor derived from the breast cancer comprises one or more cells that express PD-L1, PD-L2, or both PD-L1 and PD-L2.

In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express PD-1. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of T-cells from the subject express PD-1. In some embodiments, the percentage of cells that express PD-1 is determined using immunohistochemistry (IHC). In some embodiments, the percentage of cells that express PD-1 is determined using flow cytometry. In some embodiments, the percentage of cells that express PD-1 is determined using an enzyme-linked immunosorbent assay (ELISA).

### B. Cervical Cancer

Cervical cancer remains to be one of the leading causes of cancer-related death in women despite advances in screening, diagnosis, prevention, and treatment. It accounts for ˜4% of the total newly diagnosed cancer cases and 4% of the total cancer deaths. See Zhu et al., 2016, Drug Des. Devel. Ther. 10:1885-1895. Cervical cancer is the 7^{th} most common female cancer worldwide and the 16¹ most common cancer in the European Union. Depending on the stage at initial presentation, cervical cancer will recur in 25-61% of women. See Tempfer et al., 2016, Oncol. Res. Treat. 39:525-533. In most cases, recurrent disease is diagnosed within 2 years of the initial treatment and may be observed in various sites. Chemotherapy is the standard treatment for these patients. See Zhu et al., 2016, Drug Des. Devel. Ther. 10:1885-1895. The median overall survival exceeds one year now, however, the five year relative survival for stage IV cervical cancer is only 15%, demonstrating the high need for improved methods of treating cervical cancer.

In some aspects, provided herein are methods for treating cervical cancer in a subject with an anti-TF antibody-drug conjugate described herein and an anti-PD-1 antibody described herein. In one aspect, the antibody-drug conjugate is tisotumab vedotin. In some embodiments, the anti-PD-1 antibody or antigen-binding fragment thereof comprises the complementary determining regions (CDRs) of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof comprises the CDRs of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003. In some embodiments, the anti-PD-1 antibody or antigen-binding fragment thereof comprises a heavy chain variable region and a light chain variable region, wherein the heavy chain variable region comprises:
(i) a CDR-H1 comprising the amino acid sequence of SEQ ID NO:17;
(ii) a CDR-H2 comprising the amino acid sequence of SEQ ID NO:18; and
(iii) a CDR-H3 comprising the amino acid sequence of SEQ ID NO:19; and wherein the light chain variable region comprises:
(i) a CDR-L1 comprising the amino acid sequence of SEQ ID NO:20;
(ii) a CDR-L2 comprising the amino acid sequence of SEQ ID NO:21; and
(iii) a CDR-L3 comprising the amino acid sequence of SEQ ID NO:22. In some embodiments, the CDRs of the anti-PD-1 antibody are delineated using the Kabat numbering scheme. In one aspect, the anti-PD-1 antibody is nivolumab. In some embodiments, the subject has not previously received prior systemic therapy for the cervical cancer. In some embodiments, chemotherapy is not considered a prior systemic therapy for the cervical cancer. In some embodiments, radiation therapy is not considered a prior systemic therapy for the cervical cancer. In some embodiments, chemotherapy in combination with radiation therapy is not considered a prior systemic therapy for the cervical cancer. In some embodiments, the subject has been previously treated with chemotherapy and/or radiation therapy. In some embodiments, the subject is not a candidate for curative therapy. In some embodiments, the curative therapy is radiotherapy and/or exenterative therapy. In some embodiments, the curative therapy is radiotherapy. In some embodiments, the curative therapy is exenterative therapy. In a particular embodiment, the subject is a human.

In some embodiments of the methods or uses or product for uses provided herein, the cervical cancer is an adenocarcinoma, an adenosquamous carcinoma, a squamous cell carcinoma, a small cell carcinoma, a neuroendocrine tumor, a glassy cell carcinoma or a villoglandular adenocarcinoma. In some embodiments, the cervical cancer is an adenocarcinoma, an adenosquamous carcinoma or a squamous cell carcinoma. In some embodiments, the cervical cancer is an adenocarcinoma. In some embodiments, the cervical cancer is an adenosquamous carcinoma. In some embodiments, the cervical cancer is a squamous cell carcinoma.

In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cervical cancer cells from the subject express TF. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of the cervical cancer cells from the subject express TF. In some embodiments, the percentage of cells that express TF is determined using immunohistochemistry (IHC). In some embodiments, the percentage of cells that express TF is determined using flow cytometry. In some embodiments, the percentage of cells that express TF is determined using an enzyme-linked immunosorbent assay (ELISA).

In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cervical cancer cells from the subject express PD-L1. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of the cervical cancer cells from the subject express PD-L1. In some embodiments, the percentage of cells that express PD-L1 is determined using immunohistochemistry (IHC). In some embodiments, the percentage of cells that express PD-L1 is determined using flow cytometry. In some embodiments, the percentage of cells that express PD-L1 is determined using an enzyme-linked immunosorbent assay (ELISA).

In some embodiments, a tumor derived from the cervical cancer comprises one or more cells that express PD-L1, PD-L2, or both PD-L1 and PD-L2.

In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express PD-1. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of T-cells from the subject express PD-1. In some embodiments, the percentage of cells that express PD-1 is determined using immunohistochemistry (IHC). In some embodiments, the percentage of cells that express PD-1 is determined using flow cytometry. In some embodiments, the percentage of cells that express PD-1 is determined using an enzyme-linked immunosorbent assay (ELISA).

In some embodiments of the methods or uses or product for uses provided herein, the cervical cancer is a stage 0, 1, 2, 3, or 4 cervical cancer. In some embodiments, the cervical cancer is a stage 0, 1A, 1B, 2A, 2B, 3A, 3B, 4A or 4B cervical cancer. In some embodiments, the cervical cancer is staged by the International Federation of Gynecology and Obstetrics (FIGO) staging system. In some embodiments, the staging is based on clinical examination. In some embodiments, in stage 0 cervical cancer the carcinoma is confined to the surface layer (cells lining) the cervix. In some embodiments, in stage 1 cervical cancer the carcinoma has grown deeper into the cervix but has not yet spread beyond it. In some embodiments, in stage 1A cervical cancer the invasive carcinoma can be diagnosed only by microscopy and the deepest invasion is less than 5 mm and the largest extension is less than 7 mm. In some embodiments, in stage 1B cervical cancer the lesions are clinically visible and are limited to the cervix uteri. In some embodiments, in stage 2 cervical cancer the cervical carcinoma has invaded beyond the uterus, but not to the pelvic wall or to the lower third of the vagina. In some embodiments, in stage 2A cervical cancer there is no parametrial invasion. In some embodiments, in stage 2B cervical cancer there is parametrial invasion. In some embodiments, in stage 3 cervical cancer the tumor extends to the pelvic wall and/or involves the lower third of the vagina and/or causes hydronephrosis or non-functioning kidney. In some embodiments, in stage 3A cervical cancer the tumor involves the lower third of the vagina, with no extension to the pelvic wall. In some embodiments, in stage 3B cervical cancer extends to the pelvic wall and/or cause hydronephrosis or non-functioning kidney. In some embodiments, in stage 4 cervical cancer, the carcinoma has extended beyond the true pelvis or has involved the mucosa of the bladder or rectum. In some embodiments, in stage 4A cervical cancer the tumor has spread to adjacent organs. In some embodiments, in stage 4B cervical cancer the tumor has spread to distant organs. In some embodiments, the cervical cancer is an advanced stage cervical cancer. In some embodiments, the advanced stage cervical cancer is a grade 3 or grade 4 cervical cancer. In some embodiments, the advanced stage cervical cancer is metastatic cervical cancer. In some embodiments, the cervical cancer is metastatic and recurrent cervical cancer. In some embodiments, the cervical cancer is metastatic cervical cancer. In some embodiments, the cervical cancer is recurrent cervical cancer.

In some embodiments of the methods or uses or product for uses provided herein, the subject has not received prior systemic therapy for the cervical cancer. In some embodiments, chemotherapy is not considered a prior systemic therapy for the cervical cancer. In some embodiments, radiation therapy is not considered a prior systemic therapy for the cervical cancer. In some embodiments, chemotherapy in combination with radiation therapy is not considered a prior systemic therapy for the cervical cancer. In some embodiments, the subject has been previously treated with chemotherapy and/or radiation therapy. In some embodiments, the subject did not respond to the treatment with chemotherapy and radiation therapy. In some embodiments, the subject received treatment for the cervical cancer with chemotherapy and did not respond to the chemotherapy. In some embodiments, the subject received treatment for the cervical cancer with irradiation and did not respond to the irradiation. In some embodiments, the subject relapsed after treatment with chemotherapy and radiation therapy. In some embodiments, the subject received treatment for the cervical cancer with chemotherapy and relapsed after treatment with the chemotherapy. In some embodiments, the subject received treatment for the cervical cancer with irradiation and relapsed after treatment with irradiation. In some embodiments, the subject experienced disease progression after treatment with chemotherapy and/or radiation therapy. In some embodiments, the subject received treatment for the cervical cancer with chemotherapy and experienced disease progression after treatment with the chemotherapy. In some embodiments, the subject received treatment for the cervical cancer with irradiation and experienced disease progression after treatment with irradiation. In some embodiments, the subject is not a candidate for curative therapy. In some embodiments, the curative therapy is radiotherapy and/or exenterative therapy. In some embodiments, the curative therapy is radiotherapy. In some embodiments, the curative therapy is exenterative therapy. In a particular embodiment, the subject is a human.

### C. Routes of Administration

An anti-PD-1 antibody or antigen-binding fragment thereof described herein or anti-TF antibody-drug conjugate or antigen-binding fragment thereof described herein can be administered by any suitable route and mode. Suitable routes of administering antibodies and/or antibody-drug conjugate of the present invention are well known in the art and may be selected by those of ordinary skill in the art. In one embodiment, the anti-PD-1 antibody described herein and/or anti-TF antibody-drug conjugate described herein are administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In some embodiments, the route of administration of an anti-TF antibody-drug conjugate or antigen-binding fragment described herein is intravenous injection or infusion. In some embodiments, the route of administration of an anti-TF antibody-drug conjugate or antigen-binding fragment described herein is intravenous infusion. In some embodiments, the route of administration of an anti-PD-1 antibody or antigen-binding fragment described herein is intravenous injection or infusion. In some embodiments, the route of administration of an anti-PD-1 antibody or antigen-binding fragment described herein is intravenous infusion.

### D. Dosage and Frequency of Administration

In one aspect, the present invention provides for methods of treating a subject with cancer as described herein with a particular dose of an anti-TF antibody-drug conjugate or antigen-binding fragment thereof as described herein and an anti-PD-1 antibody or antigen-binding fragment thereof as described herein, wherein the subject is administered the antibody-drug conjugate or antigen-binding fragment thereof as described herein and the anti-PD-1 antibody or antigen-binding fragment thereof as described herein with particular frequencies.

In one embodiment of the methods or uses or product for uses provided herein, an anti-TF antibody-drug conjugate or antigen-binding fragment thereof as described herein is administered to the subject at a dose ranging from about 0.9 mg/kg to about 2.1 mg/kg of the subject's body weight. In certain embodiments, the dose is about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg or about 2.1 mg/kg. In some embodiments of the methods or uses or product for uses provided herein, an anti-TF antibody-drug conjugate or antigen-binding fragment thereof as described herein is administered to the subject at a dose ranging from 0.9 mg/kg to 2.1 mg/kg of the subject's body weight. In certain embodiments, the dose is 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg or 2.1 mg/kg. In one embodiment, the dose is about 2.0 mg/kg. In one embodiment, the dose is 2.0 mg/kg. In some embodiments, the dose is 2.0 mg/kg and the anti-TF antibody-drug conjugate is tisotumab vedotin.

In one embodiment of the methods or uses or product for uses provided herein, an anti-TF antibody-drug conjugate or antigen-binding fragment thereof as described herein is administered to the subject once about every 1 to 4 weeks. In certain embodiments, an anti-TF antibody-drug conjugate or antigen-binding fragment thereof as described herein is administered once about every 1 week, once about every 2 weeks, once about every 3 weeks or once about every 4 weeks. In one embodiment, an anti-TF antibody-drug conjugate or antigen-binding fragment thereof as described herein is administered once about every 3 weeks. In one embodiment, an anti-TF antibody-drug conjugate or antigen-binding fragment thereof as described herein is administered once every 3 weeks. In some embodiments, the dose is about 0.9 mg/kg and is administered once about every 1 week. In some embodiments, the dose is about 0.9 mg/kg and is administered once about every 2 weeks. In some embodiments, the dose is about 0.9 mg/kg and is administered once about every 3 weeks. In some embodiments, the dose is about 0.9 mg/kg and is administered once about every 4 weeks. In some embodiments, the dose is about 1.0 mg/kg and is administered once about every 1 week. In some embodiments, the dose is about 1.0 mg/kg and is administered once about every 2 weeks. In some embodiments, the dose is about 1.0 mg/kg and is administered once about every 3 weeks. In some embodiments, the dose is about 1.0 mg/kg and is administered once about every 4 weeks. In some embodiments, the dose is about 1.1 mg/kg and is administered once about every 1 week. In some embodiments, the dose is about 1.1 mg/kg and is administered once about every 2 weeks. In some embodiments, the dose is about 1.1 mg/kg and is administered once about every 3 weeks. In some embodiments, the dose is about 1.1 mg/kg and is administered once about every 4 weeks. In some embodiments, the dose is about 1.2 mg/kg and is administered once about every 1 week. In some embodiments, the dose is about 1.2 mg/kg and is administered once about every 2 weeks. In some embodiments, the dose is about 1.2 mg/kg and is administered once about every 3 weeks. In some embodiments, the dose is about 1.2 mg/kg and is administered once about every 4 weeks. In some embodiments, the dose is about 1.3 mg/kg and is administered once about every 1 week. In some embodiments, the dose is about 1.3 mg/kg and is administered once about every 2 weeks. In some embodiments, the dose is about 1.3 mg/kg and is administered once about every 3 weeks. In some embodiments, the dose is about 1.3 mg/kg and is administered once about every 4 weeks. In some embodiments, the dose is about 1.4 mg/kg and is administered once about every 1 week. In some embodiments, the dose is about 1.4 mg/kg and is administered once about every 2 weeks. In some embodiments, the dose is about 1.4 mg/kg and is administered once about every 3 weeks. In some embodiments, the dose is about 1.4 mg/kg and is administered once about every 4 weeks. In some embodiments, the dose is about 1.5 mg/kg and is administered once about every 1 week. In some embodiments, the dose is about 1.5 mg/kg and is administered once about every 2 weeks. In some embodiments, the dose is about 1.5 mg/kg and is administered once about every 3 weeks. In some embodiments, the dose is about 1.5 mg/kg and is administered once about every 4 weeks. In some embodiments, the dose is about 1.6 mg/kg and is administered once about every 1 week. In some embodiments, the dose is about 1.6 mg/kg and is administered once about every 2 weeks. In some embodiments, the dose is about 1.6 mg/kg and is administered once about every 3 weeks. In some embodiments, the dose is about 1.6 mg/kg and is administered once about every 4 weeks. In some embodiments, the dose is about 1.7 mg/kg and is administered once about every 1 week. In some embodiments, the dose is about 1.7 mg/kg and is administered once about every 2 weeks. In some embodiments, the dose is about 1.7 mg/kg and is administered once about every 3 weeks. In some embodiments, the dose is about 1.7 mg/kg and is administered once about every 4 weeks. In some embodiments, the dose is about 1.8 mg/kg and is administered once about every 1 week. In some embodiments, the dose is about 1.8 mg/kg and is administered once about every 2 weeks. In some embodiments, the dose is about 1.8 mg/kg and is administered once about every 3 weeks. In some embodiments, the dose is about 1.8 mg/kg and is administered once about every 4 weeks. In some embodiments, the dose is about 1.9 mg/kg and is administered once about every 1 week. In some embodiments, the dose is about 1.9 mg/kg and is administered once about every 2 weeks. In some embodiments, the dose is about 1.9 mg/kg and is administered once about every 3 weeks. In some embodiments, the dose is about 1.9 mg/kg and is administered once about every 4 weeks. In some embodiments, the dose is about 2.0 mg/kg and is administered once about every 1 week. In some embodiments, the dose is about 2.0 mg/kg and is administered once about every 2 weeks. In some embodiments, the dose is about 2.0 mg/kg and is administered once about every 3 weeks. In some embodiments, the dose is about 2.0 mg/kg and is administered once about every 4 weeks. In some embodiments, the dose is about 2.1 mg/kg and is administered once about every 1 week. In some embodiments, the dose is about 2.1 mg/kg and is administered once about every 2 weeks. In some embodiments, the dose is about 2.1 mg/kg and is administered once about every 3 weeks. In some embodiments, the dose is about 2.1 mg/kg and is administered once about every 4 weeks. In some embodiments, the dose is 0.9 mg/kg and is administered once about every 1 week. In some embodiments, the dose is 0.9 mg/kg and is administered once about every 2 weeks. In some embodiments, the dose is 0.9 mg/kg and is administered once about every 3 weeks. In some embodiments, the dose is 0.9 mg/kg and is administered once about every 4 weeks. In some embodiments, the dose is 1.0 mg/kg and is administered once about every 1 week. In some embodiments, the dose is 1.0 mg/kg and is administered once about every 2 weeks. In some embodiments, the dose is 1.0 mg/kg and is administered once about every 3 weeks. In some embodiments, the dose is 1.0 mg/kg and is administered once about every 4 weeks. In some embodiments, the dose is 1.1 mg/kg and is administered once about every 1 week. In some embodiments, the dose is 1.1 mg/kg and is administered once about every 2 weeks. In some embodiments, the dose is 1.1 mg/kg and is administered once about every 3 weeks. In some embodiments, the dose is 1.1 mg/kg and is administered once about every 4 weeks. In some embodiments, the dose is 1.2 mg/kg and is administered once about every 1 week. In some embodiments, the dose is 1.2 mg/kg and is administered once about every 2 weeks. In some embodiments, the dose is 1.2 mg/kg and is administered once about every 3 weeks. In some embodiments, the dose is 1.2 mg/kg and is administered once about every 4 weeks. In some embodiments, the dose is 1.3 mg/kg and is administered once about every 1 week. In some embodiments, the dose is 1.3 mg/kg and is administered once about every 2 weeks. In some embodiments, the dose is 1.3 mg/kg and is administered once about every 3 weeks. In some embodiments, the dose is 1.3 mg/kg and is administered once about every 4 weeks. In some embodiments, the dose is 1.4 mg/kg and is administered once about every 1 week. In some embodiments, the dose is 1.4 mg/kg and is administered once about every 2 weeks. In some embodiments, the dose is 1.4 mg/kg and is administered once about every 3 weeks. In some embodiments, the dose is 1.4 mg/kg and is administered once about every 4 weeks. In some embodiments, the dose is 1.5 mg/kg and is administered once about every 1 week. In some embodiments, the dose is 1.5 mg/kg and is administered once about every 2 weeks. In some embodiments, the dose is 1.5 mg/kg and is administered once about every 3 weeks. In some embodiments, the dose is 1.5 mg/kg and is administered once about every 4 weeks. In some embodiments, the dose is 1.6 mg/kg and is administered once about every 1 week. In some embodiments, the dose is 1.6 mg/kg and is administered once about every 2 weeks. In some embodiments, the dose is 1.6 mg/kg and is administered once about every 3 weeks. In some embodiments, the dose is 1.6 mg/kg and is administered once about every 4 weeks. In some embodiments, the dose is 1.7 mg/kg and is administered once about every 1 week. In some embodiments, the dose is 1.7 mg/kg and is administered once about every 2 weeks. In some embodiments, the dose is 1.7 mg/kg and is administered once about every 3 weeks. In some embodiments, the dose is 1.7 mg/kg and is administered once about every 4 weeks. In some embodiments, the dose is 1.8 mg/kg and is administered once about every 1 week. In some embodiments, the dose is 1.8 mg/kg and is administered once about every 2 weeks. In some embodiments, the dose is 1.8 mg/kg and is administered once about every 3 weeks. In some embodiments, the dose is 1.8 mg/kg and is administered once about every 4 weeks. In some embodiments, the dose is 1.9 mg/kg and is administered once about every 1 week. In some embodiments, the dose is 1.9 mg/kg and is administered once about every 2 weeks. In some embodiments, the dose is 1.9 mg/kg and is administered once about every 3 weeks. In some embodiments, the dose is 1.9 mg/kg and is administered once about every 4 weeks. In some embodiments, the dose is 2.0 mg/kg and is administered once about every 1 week. In some embodiments, the dose is 2.0 mg/kg and is administered once about every 2 weeks. In some embodiments, the dose is 2.0 mg/kg and is administered once about every 3 weeks. In some embodiments, the dose is 2.0 mg/kg and is administered once about every 4 weeks. In some embodiments, the dose is 2.1 mg/kg and is administered once about every 1 week. In some embodiments, the dose is 2.1 mg/kg and is administered once about every 2 weeks. In some embodiments, the dose is 2.1 mg/kg and is administered once about every 3 weeks. In some embodiments, the dose is 2.1 mg/kg and is administered once about every 4 weeks. In some embodiments, the dose is 2.0 mg/kg and is administered once about every 3 weeks (e.g., ±3 days). In some embodiments, the dose is 2.0 mg/kg and is administered once every 3 weeks. In some embodiments, the dose is 2.0 mg/kg and is administered once every 3 weeks and the antibody-drug conjugate is tisotumab vedotin. In some embodiments, the dose of the antibody-drug conjugate is modified if one or more adverse events occur. In some embodiments, the dose is 2.0 mg/kg and is administered once every 3 weeks and the antibody-drug conjugate is tisotumab vedotin and the dose is decreased to 1.3 mg/kg if one or more adverse events occur. In some embodiments, the dose is 1.3 mg/kg and is administered once every 3 weeks and the antibody-drug conjugate is tisotumab vedotin and the dose is decreased to 0.9 mg/kg if one or more adverse events occur.

In one embodiment of the methods or uses or product for uses provided herein, an anti-TF antibody-drug conjugate or antigen-binding fragment thereof as described herein is administered to the subject at a flat dose ranging from about 50 mg to about 200 mg such as at a flat dose of about 50 mg or a flat dose of about 60 mg or a flat dose of about 70 mg or a flat dose of about 80 mg or a flat dose of about 90 mg or a flat dose of about 100 mg or a flat dose of about 110 mg or a flat dose of about 120 mg or a flat dose of about 130 mg or a flat dose of about 140 mg or a flat dose of about 150 mg or a flat dose of about 160 mg or a flat dose of about 170 mg or a flat dose of about 180 mg or a flat dose of about 190 mg or a flat dose of about 200 mg. In some embodiments, the flat dose is administered to the subject once about every 1 to 4 weeks. In certain embodiments, the flat dose is administered to the subject once about every 1 week, once about every 2 weeks, once about every 3 weeks or once about every 4 weeks. In some embodiments, the flat dose is administered to the subject once about every 3 weeks (e.g., ±3 days). In some embodiments, the flat dose is administered to the subject once every 3 weeks. In some embodiments, the flat dose is administered to the subject once every 3 weeks and the antibody-drug conjugate is tisotumab vedotin.

In one embodiment of the methods or uses or product for uses provided herein, an anti-TF antibody-drug conjugate or antigen-binding fragment thereof as described herein is administered to the subject at a flat dose ranging from 50 mg to 200 mg such as at a flat dose of 50 mg or a flat dose of 60 mg or a flat dose of 70 mg or a flat dose of 80 mg or a flat dose of 90 mg or a flat dose of 100 mg or a flat dose of 110 mg or a flat dose of 120 mg or a flat dose of 130 mg or a flat dose of 140 mg or a flat dose of 150 mg or a flat dose of 160 mg or a flat dose of 170 mg or a flat dose of 180 mg or a flat dose of 190 mg or a flat dose of 200 mg. In some embodiments, the flat dose is administered to the subject once about every 1 to 4 weeks. In certain embodiments, the flat dose is administered to the subject once about every 1 week, once about every 2 weeks, once about every 3 weeks or once about every 4 weeks. In some embodiments, the flat dose is administered to the subject once about every 3 weeks (e.g., ±3 days). In some embodiments, the flat dose is administered to the subject once every 3 weeks. In some embodiments, the flat dose is administered to the subject once every 3 weeks and the antibody-drug conjugate is tisotumab vedotin.

In one embodiment of the methods or uses or product for uses provided herein, an anti-PD-1 antibody or antigen-binding fragment thereof as described herein is administered to the subject at a dose ranging from about 0.9 mg/kg to about 4.1 mg/kg of the subject's body weight. In certain embodiments, the dose is about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, or about 4.1 mg/kg. In some embodiments of the methods or uses or product for uses provided herein, an anti-PD-1 antibody or antigen-binding fragment thereof as described herein is administered to the subject at a dose ranging from 0.9 mg/kg to 4.1 mg/kg of the subject's body weight. In certain embodiments, the dose is 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, or 4.1 mg/kg. In one embodiment, the dose is about 1.0 mg/kg. In one embodiment, the dose is 1.0 mg/kg. In one embodiment, the dose is 1.0 mg/kg and the anti-PD-1 antibody or antigen-binding fragment thereof is nivolumab.

In one embodiment of the methods or uses or product for uses provided herein, an anti-PD-1 antibody or antigen-binding fragment thereof as described herein is administered to the subject once about every 1 to 4 weeks. In certain embodiments, an anti-PD-1 antibody or antigen-binding fragment thereof as described herein is administered once about every 1 week, once about every 2 weeks, once about every 3 weeks or once about every 4 weeks. In one embodiment, an anti-PD-1 antibody or antigen-binding fragment thereof as described herein is administered once about every 3 weeks. In one embodiment, an anti-PD-1 antibody or antigen-binding fragment thereof as described herein is administered once every 3 weeks. In some embodiments, the dose is about 0.9 mg/kg and is administered once about every 1 week. In some embodiments, the dose is about 0.9 mg/kg and is administered once about every 2 weeks. In some embodiments, the dose is about 0.9 mg/kg and is administered once about every 3 weeks. In some embodiments, the dose is about 0.9 mg/kg and is administered once about every 4 weeks. In some embodiments, the dose is about 1.0 mg/kg and is administered once about every 1 week. In some embodiments, the dose is about 1.0 mg/kg and is administered once about every 2 weeks. In some embodiments, the dose is about 1.0 mg/kg and is administered once about every 3 weeks. In some embodiments, the dose is about 1.0 mg/kg and is administered once about every 4 weeks. In some embodiments, the dose is about 1.1 mg/kg and is administered once about every 1 week. In some embodiments, the dose is about 1.1 mg/kg and is administered once about every 2 weeks. In some embodiments, the dose is about 1.1 mg/kg and is administered once about every 3 weeks. In some embodiments, the dose is about 1.1 mg/kg and is administered once about every 4 weeks. In some embodiments, the dose is about 1.2 mg/kg and is administered once about every 1 week. In some embodiments, the dose is about 1.2 mg/kg and is administered once about every 2 weeks. In some embodiments, the dose is about 1.2 mg/kg and is administered once about every 3 weeks. In some embodiments, the dose is about 1.2 mg/kg and is administered once about every 4 weeks. In some embodiments, the dose is about 1.3 mg/kg and is administered once about every 1 week. In some embodiments, the dose is about 1.3 mg/kg and is administered once about every 2 weeks. In some embodiments, the dose is about 1.3 mg/kg and is administered once about every 3 weeks. In some embodiments, the dose is about 1.3 mg/kg and is administered once about every 4 weeks. In some embodiments, the dose is about 1.4 mg/kg and is administered once about every 1 week. In some embodiments, the dose is about 1.4 mg/kg and is administered once about every 2 weeks. In some embodiments, the dose is about 1.4 mg/kg and is administered once about every 3 weeks. In some embodiments, the dose is about 1.4 mg/kg and is administered once about every 4 weeks. In some embodiments, the dose is about 1.5 mg/kg and is administered once about every 1 week. In some embodiments, the dose is about 1.5 mg/kg and is administered once about every 2 weeks. In some embodiments, the dose is about 1.5 mg/kg and is administered once about every 3 weeks. In some embodiments, the dose is about 1.5 mg/kg and is administered once about every 4 weeks. In some embodiments, the dose is about 1.6 mg/kg and is administered once about every 1 week. In some embodiments, the dose is about 1.6 mg/kg and is administered once about every 2 weeks. In some embodiments, the dose is about 1.6 mg/kg and is administered once about every 3 weeks. In some embodiments, the dose is about 1.6 mg/kg and is administered once about every 4 weeks. In some embodiments, the dose is about 1.7 mg/kg and is administered once about every 1 week. In some embodiments, the dose is about 1.7 mg/kg and is administered once about every 2 weeks. In some embodiments, the dose is about 1.7 mg/kg and is administered once about every 3 weeks. In some embodiments, the dose is about 1.7 mg/kg and is administered once about every 4 weeks. In some embodiments, the dose is about 1.8 mg/kg and is administered once about every 1 week. In some embodiments, the dose is about 1.8 mg/kg and is administered once about every 2 weeks. In some embodiments, the dose is about 1.8 mg/kg and is administered once about every 3 weeks. In some embodiments, the dose is about 1.8 mg/kg and is administered once about every 4 weeks. In some embodiments, the dose is about 1.9 mg/kg and is administered once about every 1 week. In some embodiments, the dose is about 1.9 mg/kg and is administered once about every 2 weeks. In some embodiments, the dose is about 1.9 mg/kg and is administered once about every 3 weeks. In some embodiments, the dose is about 1.9 mg/kg and is administered once about every 4 weeks. In some embodiments, the dose is about 2.0 mg/kg and is administered once about every 1 week. In some embodiments, the dose is about 2.0 mg/kg and is administered once about every 2 weeks. In some embodiments, the dose is about 2.0 mg/kg and is administered once about every 3 weeks. In some embodiments, the dose is about 2.0 mg/kg and is administered once about every 4 weeks. In some embodiments, the dose is about 2.1 mg/kg and is administered once about every 1 week. In some embodiments, the dose is about 2.1 mg/kg and is administered once about every 2 weeks. In some embodiments, the dose is about 2.1 mg/kg and is administered once about every 3 weeks. In some embodiments, the dose is about 2.1 mg/kg and is administered once about every 4 weeks. In some embodiments, the dose is about 2.2 mg/kg and is administered once about every 1 week. In some embodiments, the dose is about 2.2 mg/kg and is administered once about every 2 weeks. In some embodiments, the dose is about 2.2 mg/kg and is administered once about every 3 weeks. In some embodiments, the dose is about 2.2 mg/kg and is administered once about every 4 weeks. In some embodiments, the dose is about 2.3 mg/kg and is administered once about every 1 week. In some embodiments, the dose is about 2.3 mg/kg and is administered once about every 2 weeks. In some embodiments, the dose is about 2.3 mg/kg and is administered once about every 3 weeks. In some embodiments, the dose is about 2.3 mg/kg and is administered once about every 4 weeks. In some embodiments, the dose is about 2.4 mg/kg and is administered once about every 1 week. In some embodiments, the dose is about 2.4 mg/kg and is administered once about every 2 weeks. In some embodiments, the dose is about 2.4 mg/kg and is administered once about every 3 weeks. In some embodiments, the dose is about 2.4 mg/kg and is administered once about every 4 weeks. In some embodiments, the dose is about 2.5 mg/kg and is administered once about every 1 week. In some embodiments, the dose is about 2.5 mg/kg and is administered once about every 2 weeks. In some embodiments, the dose is about 2.5 mg/kg and is administered once about every 3 weeks. In some embodiments, the dose is about 2.5 mg/kg and is administered once about every 4 weeks. In some embodiments, the dose is about 2.6 mg/kg and is administered once about every 1 week. In some embodiments, the dose is about 2.6 mg/kg and is administered once about every 2 weeks. In some embodiments, the dose is about 2.6 mg/kg and is administered once about every 3 weeks. In some embodiments, the dose is about 2.6 mg/kg and is administered once about every 4 weeks. In some embodiments, the dose is about 2.7 mg/kg and is administered once about every 1 week. In some embodiments, the dose is about 2.7 mg/kg and is administered once about every 2 weeks. In some embodiments, the dose is about 2.7 mg/kg and is administered once about every 3 weeks. In some embodiments, the dose is about 2.7 mg/kg and is administered once about every 4 weeks. In some embodiments, the dose is about 2.8 mg/kg and is administered once about every 1 week. In some embodiments, the dose is about 2.8 mg/kg and is administered once about every 2 weeks. In some embodiments, the dose is about 2.8 mg/kg and is administered once about every 3 weeks. In some embodiments, the dose is about 2.8 mg/kg and is administered once about every 4 weeks. In some embodiments, the dose is about 2.9 mg/kg and is administered once about every 1 week. In some embodiments, the dose is about 2.9 mg/kg and is administered once about every 2 weeks. In some embodiments, the dose is about 2.9 mg/kg and is administered once about every 3 weeks. In some embodiments, the dose is about 2.9 mg/kg and is administered once about every 4 weeks. In some embodiments, the dose is about 3.0 mg/kg and is administered once about every 1 week. In some embodiments, the dose is about 3.0 mg/kg and is administered once about every 2 weeks. In some embodiments, the dose is about 3.0 mg/kg and is administered once about every 3 weeks. In some embodiments, the dose is about 3.0 mg/kg and is administered once about every 4 weeks. In some embodiments, the dose is about 3.1 mg/kg and is administered once about every 1 week. In some embodiments, the dose is about 3.1 mg/kg and is administered once about every 2 weeks. In some embodiments, the dose is about 3.1 mg/kg and is administered once about every 3 weeks. In some embodiments, the dose is about 3.1 mg/kg and is administered once about every 4 weeks. In some embodiments, the dose is about 3.2 mg/kg and is administered once about every 1 week. In some embodiments, the dose is about 3.2 mg/kg and is administered once about every 2 weeks. In some embodiments, the dose is about 3.2 mg/kg and is administered once about every 3 weeks. In some embodiments, the dose is about 3.2 mg/kg and is administered once about every 4 weeks. In some embodiments, the dose is about 3.3 mg/kg and is administered once about every 1 week. In some embodiments, the dose is about 3.3 mg/kg and is administered once about every 2 weeks. In some embodiments, the dose is about 3.3 mg/kg and is administered once about every 3 weeks. In some embodiments, the dose is about 3.3 mg/kg and is administered once about every 4 weeks. In some embodiments, the dose is about 3.4 mg/kg and is administered once about every 1 week. In some embodiments, the dose is about 3.4 mg/kg and is administered once about every 2 weeks. In some embodiments, the dose is about 3.4 mg/kg and is administered once about every 3 weeks. In some embodiments, the dose is about 3.4 mg/kg and is administered once about every 4 weeks. In some embodiments, the dose is about 3.5 mg/kg and is administered once about every 1 week. In some embodiments, the dose is about 3.5 mg/kg and is administered once about every 2 weeks. In some embodiments, the dose is about 3.5 mg/kg and is administered once about every 3 weeks. In some embodiments, the dose is about 3.5 mg/kg and is administered once about every 4 weeks. In some embodiments, the dose is about 3.6 mg/kg and is administered once about every 1 week. In some embodiments, the dose is about 3.6 mg/kg and is administered once about every 2 weeks. In some embodiments, the dose is about 3.6 mg/kg and is administered once about every 3 weeks. In some embodiments, the dose is about 3.6 mg/kg and is administered once about every 4 weeks. In some embodiments, the dose is about 3.7 mg/kg and is administered once about every 1 week. In some embodiments, the dose is about 3.7 mg/kg and is administered once about every 2 weeks. In some embodiments, the dose is about 3.7 mg/kg and is administered once about every 3 weeks. In some embodiments, the dose is about 3.7 mg/kg and is administered once about every 4 weeks. In some embodiments, the dose is about 3.8 mg/kg and is administered once about every 1 week. In some embodiments, the dose is about 3.8 mg/kg and is administered once about every 2 weeks. In some embodiments, the dose is about 3.8 mg/kg and is administered once about every 3 weeks. In some embodiments, the dose is about 3.8 mg/kg and is administered once about every 4 weeks. In some embodiments, the dose is about 3.9 mg/kg and is administered once about every 1 week. In some embodiments, the dose is about 3.9 mg/kg and is administered once about every 2 weeks. In some embodiments, the dose is about 3.9 mg/kg and is administered once about every 3 weeks. In some embodiments, the dose is about 3.9 mg/kg and is administered once about every 4 weeks. In some embodiments, the dose is about 4.0 mg/kg and is administered once about every 1 week. In some embodiments, the dose is about 4.0 mg/kg and is administered once about every 2 weeks. In some embodiments, the dose is about 4.0 mg/kg and is administered once about every 3 weeks. In some embodiments, the dose is about 4.0 mg/kg and is administered once about every 4 weeks. In some embodiments, the dose is about 4.1 mg/kg and is administered once about every 1 week. In some embodiments, the dose is about 4.1 mg/kg and is administered once about every 2 weeks. In some embodiments, the dose is about 4.1 mg/kg and is administered once about every 3 weeks. In some embodiments, the dose is about 4.1 mg/kg and is administered once about every 4 weeks. In some embodiments, the dose is 0.9 mg/kg and is administered once about every 1 week. In some embodiments, the dose is 0.9 mg/kg and is administered once about every 2 weeks. In some embodiments, the dose is 0.9 mg/kg and is administered once about every 3 weeks. In some embodiments, the dose is 0.9 mg/kg and is administered once about every 4 weeks. In some embodiments, the dose is 1.0 mg/kg and is administered once about every 1 week. In some embodiments, the dose is 1.0 mg/kg and is administered once about every 2 weeks. In some embodiments, the dose is 1.0 mg/kg and is administered once about every 3 weeks. In some embodiments, the dose is 1.0 mg/kg and is administered once about every 4 weeks. In some embodiments, the dose is 1.1 mg/kg and is administered once about every 1 week. In some embodiments, the dose is 1.1 mg/kg and is administered once about every 2 weeks. In some embodiments, the dose is 1.1 mg/kg and is administered once about every 3 weeks. In some embodiments, the dose is 1.1 mg/kg and is administered once about every 4 weeks. In some embodiments, the dose is 1.2 mg/kg and is administered once about every 1 week. In some embodiments, the dose is 1.2 mg/kg and is administered once about every 2 weeks. In some embodiments, the dose is 1.2 mg/kg and is administered once about every 3 weeks. In some embodiments, the dose is 1.2 mg/kg and is administered once about every 4 weeks. In some embodiments, the dose is 1.3 mg/kg and is administered once about every 1 week. In some embodiments, the dose is 1.3 mg/kg and is administered once about every 2 weeks. In some embodiments, the dose is 1.3 mg/kg and is administered once about every 3 weeks. In some embodiments, the dose is 1.3 mg/kg and is administered once about every 4 weeks. In some embodiments, the dose is 1.4 mg/kg and is administered once about every 1 week. In some embodiments, the dose is 1.4 mg/kg and is administered once about every 2 weeks. In some embodiments, the dose is 1.4 mg/kg and is administered once about every 3 weeks. In some embodiments, the dose is 1.4 mg/kg and is administered once about every 4 weeks. In some embodiments, the dose is 1.5 mg/kg and is administered once about every 1 week. In some embodiments, the dose is 1.5 mg/kg and is administered once about every 2 weeks. In some embodiments, the dose is 1.5 mg/kg and is administered once about every 3 weeks. In some embodiments, the dose is 1.5 mg/kg and is administered once about every 4 weeks. In some embodiments, the dose is 1.6 mg/kg and is administered once about every 1 week. In some embodiments, the dose is 1.6 mg/kg and is administered once about every 2 weeks. In some embodiments, the dose is 1.6 mg/kg and is administered once about every 3 weeks. In some embodiments, the dose is 1.6 mg/kg and is administered once about every 4 weeks. In some embodiments, the dose is 1.7 mg/kg and is administered once about every 1 week. In some embodiments, the dose is 1.7 mg/kg and is administered once about every 2 weeks. In some embodiments, the dose is 1.7 mg/kg and is administered once about every 3 weeks. In some embodiments, the dose is 1.7 mg/kg and is administered once about every 4 weeks. In some embodiments, the dose is 1.8 mg/kg and is administered once about every 1 week. In some embodiments, the dose is 1.8 mg/kg and is administered once about every 2 weeks. In some embodiments, the dose is 1.8 mg/kg and is administered once about every 3 weeks. In some embodiments, the dose is 1.8 mg/kg and is administered once about every 4 weeks. In some embodiments, the dose is 1.9 mg/kg and is administered once about every 1 week. In some embodiments, the dose is 1.9 mg/kg and is administered once about every 2 weeks. In some embodiments, the dose is 1.9 mg/kg and is administered once about every 3 weeks. In some embodiments, the dose is 1.9 mg/kg and is administered once about every 4 weeks. In some embodiments, the dose is 2.0 mg/kg and is administered once about every 1 week. In some embodiments, the dose is 2.0 mg/kg and is administered once about every 2 weeks. In some embodiments, the dose is 2.0 mg/kg and is administered once about every 3 weeks. In some embodiments, the dose is 2.0 mg/kg and is administered once about every 4 weeks. In some embodiments, the dose is 2.1 mg/kg and is administered once about every 1 week. In some embodiments, the dose is 2.1 mg/kg and is administered once about every 2 weeks. In some embodiments, the dose is 2.1 mg/kg and is administered once about every 3 weeks. In some embodiments, the dose is 2.1 mg/kg and is administered once about every 4 weeks. In some embodiments, the dose is 2.2 mg/kg and is administered once about every 1 week. In some embodiments, the dose is 2.2 mg/kg and is administered once about every 2 weeks. In some embodiments, the dose is 2.2 mg/kg and is administered once about every 3 weeks. In some embodiments, the dose is 2.2 mg/kg and is administered once about every 4 weeks. In some embodiments, the dose is 2.3 mg/kg and is administered once about every 1 week. In some embodiments, the dose is 2.3 mg/kg and is administered once about every 2 weeks. In some embodiments, the dose is 2.3 mg/kg and is administered once about every 3 weeks. In some embodiments, the dose is 2.3 mg/kg and is administered once about every 4 weeks. In some embodiments, the dose is 2.4 mg/kg and is administered once about every 1 week. In some embodiments, the dose is 2.4 mg/kg and is administered once about every 2 weeks. In some embodiments, the dose is 2.4 mg/kg and is administered once about every 3 weeks. In some embodiments, the dose is 2.4 mg/kg and is administered once about every 4 weeks. In some embodiments, the dose is 2.5 mg/kg and is administered once about every 1 week. In some embodiments, the dose is 2.5 mg/kg and is administered once about every 2 weeks. In some embodiments, the dose is 2.5 mg/kg and is administered once about every 3 weeks. In some embodiments, the dose is 2.5 mg/kg and is administered once about every 4 weeks. In some embodiments, the dose is 2.6 mg/kg and is administered once about every 1 week. In some embodiments, the dose is 2.6 mg/kg and is administered once about every 2 weeks. In some embodiments, the dose is 2.6 mg/kg and is administered once about every 3 weeks. In some embodiments, the dose is 2.6 mg/kg and is administered once about every 4 weeks. In some embodiments, the dose is 2.7 mg/kg and is administered once about every 1 week. In some embodiments, the dose is 2.7 mg/kg and is administered once about every 2 weeks. In some embodiments, the dose is 2.7 mg/kg and is administered once about every 3 weeks. In some embodiments, the dose is 2.7 mg/kg and is administered once about every 4 weeks. In some embodiments, the dose is 2.8 mg/kg and is administered once about every 1 week. In some embodiments, the dose is 2.8 mg/kg and is administered once about every 2 weeks. In some embodiments, the dose is 2.8 mg/kg and is administered once about every 3 weeks. In some embodiments, the dose is 2.8 mg/kg and is administered once about every 4 weeks. In some embodiments, the dose is 2.9 mg/kg and is administered once about every 1 week. In some embodiments, the dose is 2.9 mg/kg and is administered once about every 2 weeks. In some embodiments, the dose is 2.9 mg/kg and is administered once about every 3 weeks. In some embodiments, the dose is 2.9 mg/kg and is administered once about every 4 weeks. In some embodiments, the dose is 3.0 mg/kg and is administered once about every 1 week. In some embodiments, the dose is 3.0 mg/kg and is administered once about every 2 weeks. In some embodiments, the dose is 3.0 mg/kg and is administered once about every 3 weeks. In some embodiments, the dose is 3.0 mg/kg and is administered once about every 4 weeks. In some embodiments, the dose is 3.1 mg/kg and is administered once about every 1 week. In some embodiments, the dose is 3.1 mg/kg and is administered once about every 2 weeks. In some embodiments, the dose is 3.1 mg/kg and is administered once about every 3 weeks. In some embodiments, the dose is 3.1 mg/kg and is administered once about every 4 weeks. In some embodiments, the dose is 3.2 mg/kg and is administered once about every 1 week. In some embodiments, the dose is 3.2 mg/kg and is administered once about every 2 weeks. In some embodiments, the dose is 3.2 mg/kg and is administered once about every 3 weeks. In some embodiments, the dose is 3.2 mg/kg and is administered once about every 4 weeks. In some embodiments, the dose is 3.3 mg/kg and is administered once about every 1 week. In some embodiments, the dose is 3.3 mg/kg and is administered once about every 2 weeks. In some embodiments, the dose is 3.3 mg/kg and is administered once about every 3 weeks. In some embodiments, the dose is 3.3 mg/kg and is administered once about every 4 weeks. In some embodiments, the dose is 3.4 mg/kg and is administered once about every 1 week. In some embodiments, the dose is 3.4 mg/kg and is administered once about every 2 weeks. In some embodiments, the dose is 3.4 mg/kg and is administered once about every 3 weeks. In some embodiments, the dose is 3.4 mg/kg and is administered once about every 4 weeks. In some embodiments, the dose is 3.5 mg/kg and is administered once about every 1 week. In some embodiments, the dose is 3.5 mg/kg and is administered once about every 2 weeks. In some embodiments, the dose is 3.5 mg/kg and is administered once about every 3 weeks. In some embodiments, the dose is 3.5 mg/kg and is administered once about every 4 weeks. In some embodiments, the dose is 3.6 mg/kg and is administered once about every 1 week. In some embodiments, the dose is 3.6 mg/kg and is administered once about every 2 weeks. In some embodiments, the dose is 3.6 mg/kg and is administered once about every 3 weeks. In some embodiments, the dose is 3.6 mg/kg and is administered once about every 4 weeks. In some embodiments, the dose is 3.7 mg/kg and is administered once about every 1 week. In some embodiments, the dose is 3.7 mg/kg and is administered once about every 2 weeks. In some embodiments, the dose is 3.7 mg/kg and is administered once about every 3 weeks. In some embodiments, the dose is 3.7 mg/kg and is administered once about every 4 weeks. In some embodiments, the dose is 3.8 mg/kg and is administered once about every 1 week. In some embodiments, the dose is 3.8 mg/kg and is administered once about every 2 weeks. In some embodiments, the dose is 3.8 mg/kg and is administered once about every 3 weeks. In some embodiments, the dose is 3.8 mg/kg and is administered once about every 4 weeks. In some embodiments, the dose is 3.9 mg/kg and is administered once about every 1 week. In some embodiments, the dose is 3.9 mg/kg and is administered once about every 2 weeks. In some embodiments, the dose is 3.9 mg/kg and is administered once about every 3 weeks. In some embodiments, the dose is 3.9 mg/kg and is administered once about every 4 weeks. In some embodiments, the dose is 4.0 mg/kg and is administered once about every 1 week. In some embodiments, the dose is 4.0 mg/kg and is administered once about every 2 weeks. In some embodiments, the dose is 4.0 mg/kg and is administered once about every 3 weeks. In some embodiments, the dose is 4.0 mg/kg and is administered once about every 4 weeks. In some embodiments, the dose is 4.1 mg/kg and is administered once about every 1 week. In some embodiments, the dose is 4.1 mg/kg and is administered once about every 2 weeks. In some embodiments, the dose is 4.1 mg/kg and is administered once about every 3 weeks. In some embodiments, the dose is 4.1 mg/kg and is administered once about every 4 weeks. In some embodiments, the dose is 1.0 mg/kg and is administered once about every 3 weeks (e.g., ±3 days). In some embodiments, the dose is 1.0 mg/kg and is administered once every 3 weeks. In some embodiments, the dose is 1.0 mg/kg and is administered once every 3 weeks and the anti-PD-1 antibody or antigen-binding fragment thereof is nivolumab.

In one embodiment of the methods or uses or product for uses provided herein, an anti-PD-1 antibody or antigen-binding fragment thereof as described herein is administered to the subject at flat dose ranging from about 50 mg to about 500 mg such as at a flat dose of about 50 mg or a flat dose of about 60 mg or a flat dose of about 70 mg or a flat dose of about 80 mg or a flat dose of about 90 mg or a flat dose of about 100 mg or a flat dose of about 120 mg or a flat dose of about 140 mg or a flat dose of about 160 mg or a flat dose of about 180 mg or a flat dose of about 200 mg or a flat dose of about 220 mg or a flat dose of about 240 mg or a flat dose of about 260 mg or a flat dose of about 280 mg or a flat dose of about 300 mg or a flat dose of about 320 mg or a flat dose of about 340 mg or a flat dose of about 360 mg or a flat dose of about 380 mg or a flat dose of about 400 mg or a flat dose of about 420 mg or a flat dose of about 440 mg or a flat dose of about 460 mg or a flat dose of about 480 mg or a flat dose of about 500 mg. In some embodiments, the flat dose is about 240 mg. In some embodiments, the flat dose is about 480 mg. In some embodiments of the methods or uses or product for uses provided herein, an anti-PD-1 antibody or antigen-binding fragment thereof as described herein is administered to the subject at flat dose ranging from 50 mg to 500 mg such as at a flat dose of 50 mg or a flat dose of 60 mg or a flat dose of 70 mg or a flat dose of 80 mg or a flat dose of 90 mg or a flat dose of 100 mg or a flat dose of 120 mg or a flat dose of 140 mg or a flat dose of 160 mg or a flat dose of 180 mg or a flat dose of 200 mg or a flat dose of 220 mg or a flat dose of 240 mg or a flat dose of 260 mg or a flat dose of 280 mg or a flat dose of 300 mg or a flat dose of 320 mg or a flat dose of 340 mg or a flat dose of 360 mg or a flat dose of 380 mg or a flat dose of 400 mg or a flat dose of 420 mg or a flat dose of 440 mg or a flat dose of 460 mg or a flat dose of 480 mg or a flat dose of 500 mg. In some embodiments, the flat dose is 240 mg. In some embodiments, the flat dose is 480 mg. In some embodiments, the flat dose is 240 mg and the anti-PD-1 antibody is nivolumab. In some embodiments, the flat dose is 480 mg and the anti-PD-1 antibody is nivolumab. In some embodiments, the flat dose is about 140 mg and is administered once about every 1 week. In some embodiments, the flat dose is about 140 mg and is administered once about every 2 weeks. In some embodiments, the flat dose is about 140 mg and is administered once about every 3 weeks. In some embodiments, the flat dose is about 140 mg and is administered once about every 4 weeks. In some embodiments, the flat dose is about 160 mg and is administered once about every 1 week. In some embodiments, the flat dose is about 160 mg and is administered once about every 2 weeks. In some embodiments, the flat dose is about 160 mg and is administered once about every 3 weeks. In some embodiments, the flat dose is about 160 mg and is administered once about every 4 weeks. In some embodiments, the flat dose is about 180 mg and is administered once about every 1 week. In some embodiments, the flat dose is about 180 mg and is administered once about every 2 weeks. In some embodiments, the flat dose is about 180 mg and is administered once about every 3 weeks. In some embodiments, the flat dose is about 180 mg and is administered once about every 4 weeks. In some embodiments, the flat dose is about 200 mg and is administered once about every 1 week. In some embodiments, the flat dose is about 200 mg and is administered once about every 2 weeks. In some embodiments, the flat dose is about 200 mg and is administered once about every 3 weeks. In some embodiments, the flat dose is about 200 mg and is administered once about every 4 weeks. In some embodiments, the flat dose is about 220 mg and is administered once about every 1 week. In some embodiments, the flat dose is about 220 mg and is administered once about every 2 weeks. In some embodiments, the flat dose is about 220 mg and is administered once about every 3 weeks. In some embodiments, the flat dose is about 220 mg and is administered once about every 4 weeks. In some embodiments, the flat dose is about 240 mg and is administered once about every 1 week. In some embodiments, the dose is about 240 mg and is administered once about every 2 weeks. In some embodiments, the flat dose is about 240 mg and is administered once about every 3 weeks. In some embodiments, the flat dose is about 240 mg and is administered once about every 4 weeks. In some embodiments, the flat dose is about 260 mg and is administered once about every 1 week. In some embodiments, the flat dose is about 260 mg and is administered once about every 2 weeks. In some embodiments, the flat dose is about 260 mg and is administered once about every 3 weeks. In some embodiments, the flat dose is about 260 mg and is administered once about every 4 weeks. In some embodiments, the flat dose is 140 mg and is administered once about every 1 week. In some embodiments, the flat dose is 140 mg and is administered once about every 2 weeks. In some embodiments, the flat dose is 140 mg and is administered once about every 3 weeks. In some embodiments, the flat dose is 140 mg and is administered once about every 4 weeks. In some embodiments, the flat dose is 160 mg and is administered once about every 1 week. In some embodiments, the flat dose is 160 mg and is administered once about every 2 weeks. In some embodiments, the flat dose is 160 mg and is administered once about every 3 weeks. In some embodiments, the flat dose is 160 mg and is administered once about every 4 weeks. In some embodiments, the flat dose is 180 mg and is administered once about every 1 week. In some embodiments, the flat dose is 180 mg and is administered once about every 2 weeks. In some embodiments, the flat dose is 180 mg and is administered once about every 3 weeks. In some embodiments, the flat dose is 180 mg and is administered once about every 4 weeks. In some embodiments, the flat dose is 200 mg and is administered once about every 1 week. In some embodiments, the flat dose is 200 mg and is administered once about every 2 weeks. In some embodiments, the flat dose is 200 mg and is administered once about every 3 weeks. In some embodiments, the flat dose is 200 mg and is administered once about every 4 weeks. In some embodiments, the flat dose is 220 mg and is administered once about every 1 week. In some embodiments, the flat dose is 220 mg and is administered once about every 2 weeks. In some embodiments, the flat dose is 220 mg and is administered once about every 3 weeks. In some embodiments, the flat dose is 220 mg and is administered once about every 4 weeks. In some embodiments, the flat dose is 240 mg and is administered once about every 1 week. In some embodiments, the flat dose is 240 mg and is administered once about every 2 weeks. In some embodiments, the flat dose is 240 mg and is administered once about every 3 weeks. In some embodiments, the flat dose is 240 mg and is administered once about every 4 weeks. In some embodiments, the flat dose is 260 mg and is administered once about every 1 week. In some embodiments, the flat dose is 260 mg and is administered once about every 2 weeks. In some embodiments, the flat dose is 260 mg and is administered once about every 3 weeks. In some embodiments, the flat dose is 260 mg and is administered once about every 4 weeks. In some embodiments, the flat dose is 240 mg and is administered once about every 2 weeks (e.g., ±2 days). In some embodiments, the flat dose is 240 mg and is administered once every 2 weeks. In some embodiments, the flat dose is 240 mg and is administered once every 2 weeks and the antibody is nivolumab. In some embodiments, the flat dose is 240 mg and is administered once about every 4 weeks (e.g., ±4 days). In some embodiments, the flat dose is 480 mg and is administered once every 4 weeks. In some embodiments, the flat dose is 480 mg and is administered once every 4 weeks and the antibody is nivolumab.

In some embodiments of the methods or uses or product for uses provided herein, an anti-PD-1 antibody or antigen-binding fragment thereof as described herein and an anti-TF antibody-drug conjugate or antigen-binding fragment thereof as described herein are administered to the subject at a fixed dose. In some embodiments, the fixed dose is based on the amount (e.g., mg) of the antibodies. In certain embodiments, the fixed dose is based on the concentration (e.g., mg/ml) of the antibodies. In some embodiments, the ratio of the amount (e.g., mg) of the anti-PD-1 antibody or antigen-binding fragment thereof as described herein to the amount (e.g., mg) of the anti-TF antibody-drug conjugate or antigen-binding fragment thereof as described herein is about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1. In some embodiments, the ratio of the amount (e.g., mg) of the anti-PD-1 antibody or antigen-binding fragment thereof as described herein to the amount (e.g., mg) of the anti-TF antibody-drug conjugate or antigen-binding fragment thereof as described herein is 1:1, 1:2, 1:3, 1:4, 1:5, 1:6, 1:7, 1:8, 1:9, 1:10, 1:15, 1:20, 1:30, 1:40, 1:50, 1:60, 1:70, 1:80, 1:90, 1:100, 1:120, 1:140, 1:160, 1:180, 1:200, 200:1, 180:1, 160:1, 140:1, 120:1, 100:1, 90:1, 80:1, 70:1, 60:1, 50:1, 40:1, 30:1, 20:1, 15:1, 10:1, 9:1, 8:1, 7:1, 6:1, 5:1, 4:1, 3:1, or 2:1. In some embodiments, the ratio of the concentration (e.g., mg/ml) of the anti-PD-1 antibody or antigen-binding fragment thereof as described herein to the concentration (e.g., mg/ml) of the anti-TF antibody-drug conjugate or antigen-binding fragment thereof as described herein is about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1. In some embodiments, the ratio of the concentration (e.g., mg/ml) of the anti-PD-1 antibody or antigen-binding fragment thereof described herein to the concentration (e.g., mg/ml) of the anti-TF antibody-drug conjugate or antigen-binding fragment thereof described herein is 1:1, 1:2, 1:3, 1:4, 1:5, 1:6, 1:7, 1:8, 1:9, 1:10, 1:15, 1:20, 1:30, 1:40, 1:50, 1:60, 1:70, 1:80, 1:90, 1:100, 1:120, 1:140, 1:160, 1:180, 1:200, 200:1, 180:1, 160:1, 140:1, 120:1, 100:1, 90:1, 80:1, 70:1, 60:1, 50:1, 40:1, 30:1, 20:1, 15:1, 10:1, 9:1, 8:1, 7:1, 6:1, 5:1, 4:1, 3:1, or 2:1.

In some embodiments, the dose of the anti-TF antibody-drug conjugate described herein is 2.0 mg/kg and is administered once about every 3 weeks (e.g., ±3 days) and the dose of the anti-PD-1 antibody described herein is 240 mg and is administered once about every 2 weeks (e.g., ±2 days). In some embodiments, the dose of the anti-TF antibody-drug conjugate described herein is 2.0 mg/kg and is administered once every 3 weeks and the dose of the anti-PD-1 antibody described herein is 240 mg and is administered once every 2 weeks. In some embodiments, the dose of the anti-TF antibody-drug conjugate is 2.0 mg/kg and is administered once every 3 weeks and the antibody-drug conjugate is tisotumab vedotin and the dose of the anti-PD-1 antibody is 240 mg and is administered once every 3 weeks and the anti-PD-1 antibody is nivolumab. In some embodiments, the dose of the anti-TF antibody-drug conjugate described herein is 2.0 mg/kg and is administered once about every 3 weeks (e.g., ±3 days) and the dose of the anti-PD-1 antibody described herein is 480 mg and is administered once about every 4 weeks (e.g., ±4 days). In some embodiments, the dose of the anti-TF antibody-drug conjugate described herein is 2.0 mg/kg and is administered once every 3 weeks and the dose of the anti-PD-1 antibody described herein is 480 mg and is administered once every 4 weeks. In some embodiments, the dose of the anti-TF antibody-drug conjugate is 2.0 mg/kg and is administered once every 3 weeks and the antibody-drug conjugate is tisotumab vedotin and the dose of the anti-PD-1 antibody is 480 mg and is administered once every 4 weeks and the anti-PD-1 antibody is nivolumab.

In some embodiments, an anti-TF antibody-drug conjugate or antigen-binding fragment thereof as described herein and an anti-PD-1 antibody or antigen-binding fragment thereof as described herein are coadministered. In some embodiments the coadministration is simultaneous or sequential. In some embodiments, an anti-TF antibody-drug conjugate as described herein is administered simultaneously with an anti-PD-1 antibody as described herein. In some embodiments, simultaneous means that the anti-TF antibody-drug conjugate as described herein and the anti-PD-1 antibody as described herein are administered to the subject less than about one hour apart, such as less than about 30 minutes apart, less than about 15 minutes apart, less than about 10 minutes apart or less than about 5 minutes apart. In some embodiments, simultaneous means that the anti-TF antibody-drug conjugate as described herein and the anti-PD-1 antibody as described herein are administered to the subject less than one hour apart, such as less than 30 minutes apart, less than 15 minutes apart, less than 10 minutes apart or less than 5 minutes apart. In some embodiments, an anti-TF antibody-drug conjugate as described herein is administered sequentially with an anti-PD-1 antibody as described herein. In some embodiments, sequential administration means that the anti-TF antibody-drug conjugate as described herein and the anti-PD-1 antibody as described herein are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart.

In some embodiments, a method of treatment or use described herein further comprises the administration of one or more additional therapeutic agents. In some embodiments, the one or more additional therapeutic agents are administered simultaneously with an anti-TF antibody-drug conjugate or antigen-binding fragment thereof as described herein, such as tisotumab vedotin, and an anti-PD-1 antibody or antigen-binding fragment thereof as described herein, such as nivolumab. In some embodiments, the one or more additional therapeutic agents and an anti-TF antibody-drug conjugate or antigen-binding fragment thereof as described herein and an anti-PD-1 antibody or antigen-binding fragment thereof as described herein are administered sequentially.

### E. Treatment Outcome

In one aspect, a method of treating cancer with an anti-TF antibody-drug conjugate or antigen-binding fragment thereof as described herein and an anti-PD-1 antibody or antigen-binding fragment thereof as described herein results in an improvement in one or more therapeutic effects in the subject after administration of the antibody-drug conjugate relative to a baseline. In some embodiments, the one or more therapeutic effects is the size of the tumor derived from the cancer (e.g., breast cancer or cervical cancer), the objective response rate, the duration of response, the time to response, progression free survival, overall survival, or any combination thereof. In one embodiment, the one or more therapeutic effects is the size of the tumor derived from the cancer. In one embodiment, the one or more therapeutic effects is decreased tumor size. In one embodiment, the one or more therapeutic effects is stable disease. In one embodiment, the one or more therapeutic effects is partial response. In one embodiment, the one or more therapeutic effects is complete response. In one embodiment, the one or more therapeutic effects is the objective response rate. In one embodiment, the one or more therapeutic effects is the duration of response. In one embodiment, the one or more therapeutic effects is the time to response. In one embodiment, the one or more therapeutic effects is progression free survival. In one embodiment, the one or more therapeutic effects is overall survival. In one embodiment, the one or more therapeutic effects is cancer regression.

In one embodiment of the methods or uses or product for uses provided herein, response to treatment with an anti-TF antibody-drug conjugate or antigen-binding fragment thereof as described herein and an anti-PD-1 antibody or antigen-binding fragment thereof as described herein may include the following criteria (RECIST Criteria 1.1):

| | Category | Criteria |
|---|---|---|
| Based on | Complete | Disappearance of all target lesions. Any pathological |
| target lesions | Response (CR) | lymph nodes must have reduction in short axis to <10 |
| | | mm. |
| | Partial Response | ≥30% decrease in the sum of the longest diameter |
| | (PR) | (LD) of target lesions, taking as reference the baseline |
| | | sum of LDs. |
| | Stable Disease | Neither sufficient shrinkage to qualify for PR nor |
| | (SD) | sufficient increase to qualify for PD, taking as |
| | | reference the smallest sum of LDs while in trial. |
| | Progressive | ≥20% (and ≥5 mm) increase in the sum of the LDs |
| | Disease (PD) | of target lesions, taking as reference the smallest sum |
| | | of the target LDs recorded while in trial or the |
| | | appearance of one or more new lesions. |
| Based on non- | CR | Disappearance of all non-target lesions and |
| target lesions | | normalization of tumor marker level. All lymph nodes |
| | | must be non-pathological in size (<10 mm short |
| | | axis). |
| | SD | Persistence of one or more non-target lesion(s) or/and |
| | | maintenance of tumor marker level above the normal |
| | | limits. |
| | PD | Appearance of one or more new lesions and/or |
| | | unequivocal progression of existing non-target |
| | | lesions. |

In one embodiment of the methods or uses or product for uses provided herein, the effectiveness of treatment with an anti-TF antibody-drug conjugate or antigen-binding fragment thereof described herein and an anti-PD-1 antibody or antigen-binding fragment thereof described herein is assessed by measuring the objective response rate. In some embodiments, the objective response rate is the proportion of patients with tumor size reduction of a predefined amount and for a minimum period of time. In some embodiments the objective response rate is based upon RECIST v1.1. In one embodiment, the objective response rate is at least about 20%, at least about 25%, at least about 30%, at least about 35%, at least about 40%, at least about 45%, at least about 50%, at least about 60%, at least about 70%, or at least about 80%. In one embodiment, the objective response rate is at least about 20%-80%. In one embodiment, the objective response rate is at least about 30%-80%. In one embodiment, the objective response rate is at least about 40%-80%. In one embodiment, the objective response rate is at least about 50%-80%. In one embodiment, the objective response rate is at least about 60%-80%. In one embodiment, the objective response rate is at least about 70%-80%. In one embodiment, the objective response rate is at least about 80%. In one embodiment, the objective response rate is at least about 85%. In one embodiment, the objective response rate is at least about 90%. In one embodiment, the objective response rate is at least about 95%. In one embodiment, the objective response rate is at least about 98%. In one embodiment, the objective response rate is at least about 99%. In one embodiment, the objective response rate is at least 20%, at least 25%, at least 30%, at least 35%, at least 40%, at least 45%, at least 50%, at least 60%, at least 70%, or at least 80%. In one embodiment, the objective response rate is at least 20%-80%. In one embodiment, the objective response rate is at least 30%-80%. In one embodiment, the objective response rate is at least 40%-80%. In one embodiment, the objective response rate is at least 50%-80%. In one embodiment, the objective response rate is at least 60%-80%. In one embodiment, the objective response rate is at least 70%-80%. In one embodiment, the objective response rate is at least 80%. In one embodiment, the objective response rate is at least 85%. In one embodiment, the objective response rate is at least 90%. In one embodiment, the objective response rate is at least 95%. In one embodiment, the objective response rate is at least 98%. In one embodiment, the objective response rate is at least 99%. In one embodiment, the objective response rate is 100%.

In one embodiment of the methods or uses or product for uses provided herein, response to treatment with an anti-TF antibody-drug conjugate or antigen-binding fragment thereof described herein and an anti-PD-1 antibody or antigen-binding fragment thereof described herein is assessed by measuring the size of a tumor derived from the cancer (e.g., breast cancer or cervical cancer). In one embodiment, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the anti-TF antibody-drug conjugate described herein and/or the anti-PD-1 antibody described herein. In one embodiment, the size of a tumor derived from the cancer is reduced by at least about 10%-80%. In one embodiment, the size of a tumor derived from the cancer is reduced by at least about 20%-80%. In one embodiment, the size of a tumor derived from the cancer is reduced by at least about 30%-80%. In one embodiment, the size of a tumor derived from the cancer is reduced by at least about 40%-80%. In one embodiment, the size of a tumor derived from the cancer is reduced by at least about 50%-80%. In one embodiment, the size of a tumor derived from the cancer is reduced by at least about 60%-80%. In one embodiment, the size of a tumor derived from the cancer is reduced by at least about 70%-80%. In one embodiment, the size of a tumor derived from the cancer is reduced by at least about 80%. In one embodiment, the size of a tumor derived from the cancer is reduced by at least about 85%. In one embodiment, the size of a tumor derived from the cancer is reduced by at least about 90%. In one embodiment, the size of a tumor derived from the cancer is reduced by at least about 95%. In one embodiment, the size of a tumor derived from the cancer is reduced by at least about 98%. In one embodiment, the size of a tumor derived from the cancer is reduced by at least about 99%. In one embodiment, the size of a tumor derived from the cancer is reduced by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of the anti-TF antibody-drug conjugate described herein and/or the anti-PD-1 antibody described herein. In one embodiment, the size of a tumor derived from the cancer is reduced by at least 10%-80%. In one embodiment, the size of a tumor derived from the cancer is reduced by at least 20%-80%. In one embodiment, the size of a tumor derived from the cancer is reduced by at least 30%-80%. In one embodiment, the size of a tumor derived from the cancer is reduced by at least 40%-80%. In one embodiment, the size of a tumor derived from the cancer is reduced by at least 50%-80%. In one embodiment, the size of a tumor derived from the cancer is reduced by at least 60%-80%. In one embodiment, the size of a tumor derived from the cancer is reduced by at least 70%-80%. In one embodiment, the size of a tumor derived from the cancer is reduced by at least 80%. In one embodiment, the size of a tumor derived from the cancer is reduced by at least 85%. In one embodiment, the size of a tumor derived from the cancer is reduced by at least 90%. In one embodiment, the size of a tumor derived from the cancer is reduced by at least 95%. In one embodiment, the size of a tumor derived from the cancer is reduced by at least 98%. In one embodiment, the size of a tumor derived from the cancer is reduced by at least 99%. In one embodiment, the size of a tumor derived from the cancer is reduced by 100%. In one embodiment, the size of a tumor derived from the cancer is measured by magnetic resonance imaging (MRI). In one embodiment, the size of a tumor derived from the cancer is measured by computed tomography (CT). In some embodiments, the size of a tumor derived from a cervical cancer is measured by pelvic examination. See Choi et al., 2008, J. Gynecol. Oncol. 19(3):205. In some embodiments, the size of a tumor derived from a breast cancer is measured by mammography, sonography or magnetic resonance imaging (MRI). See Gruber et. al., 2013, BMC Cancer. 13:328. In some embodiments, the size of the tumor derived from the cancer is reduced relative to the size of the tumor before administration of the anti-TF antibody drug conjugate described herein and the anti-PD-1 antibody described herein. In some embodiments, the size of the tumor derived from the cancer is reduced relative to the size of the tumor before administration of the anti-TF antibody drug conjugate described herein. In some embodiments, the size of the tumor derived from the cancer is reduced relative to the size of the tumor before administration of the anti-PD-1 antibody described herein.

In one embodiment of the methods or uses or product for uses provided described herein, response to treatment with an antibody-drug conjugate or antigen-binding fragment thereof described herein, such as e.g., tisotumab vedotin, and an anti-PD-1 antibody or antigen-binding fragment thereof described herein, such as e.g., nivolumab, promotes regression of a tumor derived from the cancer (e.g., breast cancer or cervical cancer). In one embodiment, a tumor derived from the cancer regresses by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the anti-TF antibody-drug conjugate described herein and/or anti-PD-1 antibody described herein. In one embodiment, a tumor derived from the cancer regresses by at least about 10% to about 80%. In one embodiment, a tumor derived from the cancer regresses by at least about 20% to about 80%. In one embodiment, a tumor derived from the cancer regresses by at least about 30% to about 80%. In one embodiment, a tumor derived from the cancer regresses by at least about 40% to about 80%. In one embodiment, a tumor derived from the cancer regresses by at least about 50% to about 80%. In one embodiment, a tumor derived from the cancer regresses by at least about 60% to about 80%. In one embodiment, a tumor derived from the cancer regresses by at least about 70% to about 80%. In one embodiment, a tumor derived from the cancer regresses by at least about 80%. In one embodiment, a tumor derived from the cancer regresses by at least about 85%. In one embodiment, a tumor derived from the cancer regresses by at least about 90%. In one embodiment, a tumor derived from the cancer regresses by at least about 95%. In one embodiment, a tumor derived from the cancer regresses by at least about 98%. In one embodiment, a tumor derived from the cancer regresses by at least about 99%. In one embodiment, a tumor derived from the cancer regresses by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of the anti-TF antibody-drug conjugate described herein and/or anti-PD-1 antibody described herein. In one embodiment, a tumor derived from the cancer regresses by at least 10% to 80%. In one embodiment, a tumor derived from the cancer regresses by at least 20% to 80%. In one embodiment, a tumor derived from the cancer regresses by at least 30% to 80%. In one embodiment, a tumor derived from the cancer regresses by at least 40% to 80%. In one embodiment, a tumor derived from the cancer regresses by at least 50% to 80%. In one embodiment, a tumor derived from the cancer regresses by at least 60% to 80%. In one embodiment, a tumor derived from the cancer regresses by at least 70% to 80%. In one embodiment, a tumor derived from the cancer regresses by at least 80%. In one embodiment, a tumor derived from the cancer regresses by at least 85%. In one embodiment, a tumor derived from the cancer regresses by at least 90%. In one embodiment, a tumor derived from the cancer regresses by at least 95%. In one embodiment, a tumor derived from the cancer regresses by at least 98%. In one embodiment, a tumor derived from the cancer regresses by at least 99%. In one embodiment, a tumor derived from the cancer regresses by 100%. In one embodiment, regression of a tumor is determined by measuring the size of the tumor by magnetic resonance imaging (MRI). In one embodiment, regression of a tumor is determined by measuring the size of the tumor by computed tomography (CT). In some embodiments, regression of a tumor is determined by measuring the size of the tumor by pelvic examination. See Choi et al., 2008, J. Gynecol. Oncol. 19(3):205. In some embodiments, regression of a tumor is determined by mammography, sonography or magnetic resonance imaging (MRI). See Gruber et. al., 2013, BMC Cancer. 13:328. In some embodiments, the tumor derived from the cancer regresses relative to the size of the tumor before administration of the anti-TF antibody drug conjugate described herein and the anti-PD-1 antibody described herein. In some embodiments, the tumor derived from the cancer regresses relative to the size of the tumor before administration of the anti-TF antibody drug conjugate described herein. In some embodiments, the tumor derived from the cancer regresses relative to the size of the tumor before administration of the anti-PD-1 antibody described herein.

In one embodiment of the methods or uses or product for uses described herein, response to treatment with an anti-TF antibody-drug conjugate or antigen-binding fragment thereof described herein and an anti-PD-1 antibody or antigen-binding fragment thereof described herein is assessed by measuring the time of progression free survival after administration of the anti-TF antibody-drug conjugate described herein and/or the anti-PD-1 antibody described herein. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-TF antibody-drug conjugate described herein and/or the anti-PD-1 antibody described herein. In some embodiments, the subject exhibits progression-free survival of at least about 6 months after administration of the anti-TF antibody-drug conjugate described herein and/or the anti-PD-1 antibody described herein. In some embodiments, the subject exhibits progression-free survival of at least about one year after administration of the anti-TF antibody-drug conjugate described herein and/or the anti-PD-1 antibody described herein. In some embodiments, the subject exhibits progression-free survival of at least about two years after administration of the anti-TF antibody-drug conjugate described herein and/or the anti-PD-1 antibody described herein. In some embodiments, the subject exhibits progression-free survival of at least about three years after administration of the anti-TF antibody-drug conjugate described herein and/or the anti-PD-1 antibody described herein. In some embodiments, the subject exhibits progression-free survival of at least about four years after administration of the anti-TF antibody-drug conjugate described herein and/or the anti-PD-1 antibody described herein. In some embodiments, the subject exhibits progression-free survival of at least about five years after administration of the anti-TF antibody-drug conjugate described herein and/or the anti-PD-1 antibody described herein. In some embodiments, the subject exhibits progression-free survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the anti-TF antibody-drug conjugate described herein and/or the anti-PD-1 antibody described herein. In some embodiments, the subject exhibits progression-free survival of at least 6 months after administration of the anti-TF antibody-drug conjugate described herein and/or the anti-PD-1 antibody described herein. In some embodiments, the subject exhibits progression-free survival of at least one year after administration of the anti-TF antibody-drug conjugate described herein and/or the anti-PD-1 antibody described herein. In some embodiments, the subject exhibits progression-free survival of at least two years after administration of the anti-TF antibody-drug conjugate described herein and/or the anti-PD-1 antibody described herein. In some embodiments, the subject exhibits progression-free survival of at least three years after administration of the anti-TF antibody-drug conjugate described herein and/or the anti-PD-1 antibody described herein. In some embodiments, the subject exhibits progression-free survival of at least four years after administration of the anti-TF antibody-drug conjugate described herein and/or the anti-PD-1 antibody described herein. In some embodiments, the subject exhibits progression-free survival of at least five years after administration of the anti-TF antibody-drug conjugate described herein and/or the anti-PD-1 antibody described herein. In some embodiments, response to treatment is assessed by measuring the time of progression free survival after administration of the anti-TF antibody-drug conjugate described herein and the anti-PD-1 antibody described herein. In some embodiments, response to treatment is assessed by measuring the time of progression free survival after administration of the anti-TF antibody-drug conjugate described herein. In some embodiments, response to treatment is assessed by measuring the time of progression free survival after administration of the anti-PD-1 antibody described herein.

In one embodiment of the methods or uses or product for uses described herein, response to treatment with an anti-TF antibody-drug conjugate or antigen-binding fragment thereof described herein and an anti-PD-1 antibody or antigen-binding fragment thereof described herein is assessed by measuring the time of overall survival after administration of the anti-TF antibody-drug conjugate described herein and/or the anti-PD-1 antibody described herein. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-TF antibody-drug conjugate described herein and/or the anti-PD-1 antibody described herein. In some embodiments, the subject exhibits overall survival of at least about 6 months after administration of the anti-TF antibody-drug conjugate described herein and/or the anti-PD-1 antibody described herein. In some embodiments, the subject exhibits overall survival of at least about one year after administration of the anti-TF antibody-drug conjugate described herein and/or the anti-PD-1 antibody described herein. In some embodiments, the subject exhibits overall survival of at least about two years after administration of the anti-TF antibody-drug conjugate described herein and/or the anti-PD-1 antibody described herein. In some embodiments, the subject exhibits overall survival of at least about three years after administration of the anti-TF antibody-drug conjugate described herein and/or the anti-PD-1 antibody described herein. In some embodiments, the subject exhibits overall survival of at least about four years after administration of the anti-TF antibody-drug conjugate described herein and/or the anti-PD-1 antibody described herein. In some embodiments, the subject exhibits overall survival of at least about five years after administration of the anti-TF antibody-drug conjugate described herein and/or the anti-PD-1 antibody described herein. In some embodiments, the subject exhibits overall survival of at least 1 month, at least 2 months, at least 3 months, at least 4 months, at least 5 months, at least 6 months, at least 7 months, at least 8 months, at least 9 months, at least 10 months, at least 11 months, at least about 12 months, at least eighteen months, at least two years, at least three years, at least four years, or at least five years after administration of the anti-TF antibody-drug conjugate described herein and/or the anti-PD-1 antibody described herein. In some embodiments, the subject exhibits overall survival of at least 6 months after administration of the anti-TF antibody-drug conjugate described herein and/or the anti-PD-1 antibody described herein. In some embodiments, the subject exhibits overall survival of at least one year after administration of the anti-TF antibody-drug conjugate described herein and/or the anti-PD-1 antibody described herein. In some embodiments, the subject exhibits overall survival of at least two years after administration of the anti-TF antibody-drug conjugate described herein and/or the anti-PD-1 antibody described herein. In some embodiments, the subject exhibits overall survival of at least three years after administration of the anti-TF antibody-drug conjugate described herein and/or the anti-PD-1 antibody described herein. In some embodiments, the subject exhibits overall survival of at least four years after administration of the anti-TF antibody-drug conjugate described herein and/or the anti-PD-1 antibody described herein. In some embodiments, the subject exhibits overall survival of at least five years after administration of the anti-TF antibody-drug conjugate described herein and/or the anti-PD-1 antibody described herein. In some embodiments, response to treatment is assessed by measuring the time of overall survival after administration of the anti-TF antibody-drug conjugate described herein and the anti-PD-1 antibody described herein. In some embodiments, response to treatment is assessed by measuring the time of overall survival after administration of the anti-TF antibody-drug conjugate described herein. In some embodiments, response to treatment is assessed by measuring the time of overall survival after administration of the anti-PD-1 antibody described herein.

In one embodiment of the methods or uses or product for uses described herein, response to treatment with an anti-TF antibody-drug conjugate or antigen-binding fragment thereof described herein and an anti-PD-1 antibody or antigen-binding fragment thereof described herein is assessed by measuring the duration of response to the anti-TF antibody-drug conjugate described herein and the anti-PD-1 antibody described herein after administration of the anti-TF antibody-drug conjugate described herein and/or the anti-PD-1 antibody described herein. In some embodiments, the duration of response to the anti-TF antibody-drug conjugate described herein and the anti-PD-1 antibody described herein is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-TF antibody-drug conjugate described herein and/or the anti-PD-1 antibody described herein. In some embodiments, the duration of response to the anti-TF antibody-drug conjugate described herein and the anti-PD-1 antibody described herein is at least about 6 months after administration of the antibody-drug conjugate described herein and/or the anti-PD-1 antibody described herein. In some embodiments, the duration of response to the anti-TF antibody-drug conjugate described herein and the anti-PD-1 antibody described herein is at least about one year after administration of the antibody-drug conjugate described herein and/or the anti-PD-1 antibody described herein. In some embodiments, the duration of response to the anti-TF antibody-drug conjugate described herein and the anti-PD-1 antibody described herein is at least about two years after administration of the antibody-drug conjugate described herein and/or the anti-PD-1 antibody described herein. In some embodiments, the duration of response to the anti-TF antibody-drug conjugate described herein and the anti-PD-1 antibody described herein is at least about three years after administration of the antibody-drug conjugate described herein and/or the anti-PD-1 antibody described herein. In some embodiments, the duration of response to the anti-TF antibody-drug conjugate described herein and the anti-PD-1 antibody described herein is at least about four years after administration of the antibody-drug conjugate described herein and/or the anti-PD-1 antibody described herein. In some embodiments, the duration of response to the anti-TF antibody-drug conjugate described herein and the anti-PD-1 antibody described herein is at least about five years after administration of the antibody-drug conjugate described herein and/or the anti-PD-1 antibody described herein. In some embodiments, the duration of response to the anti-TF antibody-drug conjugate described herein and the anti-PD-1 antibody described herein is at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the anti-TF antibody-drug conjugate described herein and/or the anti-PD-1 antibody described herein. In some embodiments, the duration of response to the anti-TF antibody-drug conjugate described herein and the anti-PD-1 antibody described herein is at least 6 months after administration of the antibody-drug conjugate described herein and/or the anti-PD-1 antibody described herein. In some embodiments, the duration of response to the anti-TF antibody-drug conjugate described herein and the anti-PD-1 antibody described herein is at least one year after administration of the antibody-drug conjugate described herein and/or the anti-PD-1 antibody described herein. In some embodiments, the duration of response to the anti-TF antibody-drug conjugate described herein and the anti-PD-1 antibody described herein is at least two years after administration of the antibody-drug conjugate described herein and/or the anti-PD-1 antibody described herein. In some embodiments, the duration of response to the anti-TF antibody-drug conjugate described herein and the anti-PD-1 antibody described herein is at least three years after administration of the antibody-drug conjugate described herein and/or the anti-PD-1 antibody described herein. In some embodiments, the duration of response to the anti-TF antibody-drug conjugate described herein and the anti-PD-1 antibody described herein is at least four years after administration of the antibody-drug conjugate described herein and/or the anti-PD-1 antibody described herein. In some embodiments, the duration of response to the anti-TF antibody-drug conjugate described herein and the anti-PD-1 antibody described herein is at least five years after administration of the antibody-drug conjugate described herein and/or the anti-PD-1 antibody described herein. In some embodiments, the duration of response is measured after administration of the anti-TF antibody drug conjugate described herein and the anti-PD-1 antibody described herein. In some embodiments, the duration of response is measured after administration of the anti-TF antibody drug conjugate described herein. In some embodiments, the duration of response is measured after administration of the anti-PD-1 antibody described herein.

### F. Adverse Events

In one aspect, a method of treating cancer (e.g., breast cancer or cervical cancer) with an anti-TF antibody-drug conjugates or antigen-binding fragments thereof described herein and an anti-PD-1 antibody or antigen-binding fragment thereof described herein results in the subject developing one or more adverse events. In some embodiments, the subject is administered an additional therapeutic agent to eliminate or reduce the severity of the adverse event. In some embodiments, the one or more adverse events the subject develops is anemia, abdominal pain, hemorrhage, hyperthyroidism, hypothyroidism, hypokalemia, hyponatremia, epistaxis, fatigue, nausea, alopecia, conjunctivitis, keratitis, conjunctival ulceration, constipation, decreased appetite, diarrhea, vomiting, peripheral neuropathy, or general physical health deterioration, or any combination thereof. In some embodiments, the one or more adverse events is a grade 1 or greater adverse event. In some embodiments, the one or more adverse events is a grade 2 or greater adverse event. In some embodiments, the one or more adverse events is a grade 3 or greater adverse event. In some embodiments, the one or more adverse events is a grade 1 adverse event. In some embodiments, the one or more adverse events is a grade 2 adverse event. In some embodiments, the one or more adverse events is a grade 3 adverse event. In some embodiments, the one or more adverse events is a grade 4 adverse event. In some embodiments, the one or more adverse events is a serious adverse event. In some embodiments, the one or more adverse events is conjunctivitis, conjunctival ulceration, and/or keratitis and the additional therapeutic agent is a preservative-free lubricating eye drop, an ocular vasoconstrictor, antibiotic, a steroid eye drop, or any combination thereof. In some embodiments, the one or more adverse events is conjunctivitis, conjunctival ulceration, and keratitis and the additional therapeutic agent is a preservative-free lubricating eye drop, an ocular vasoconstrictor, antibiotic, a steroid eye drop, or any combination thereof. In some embodiments, the one or more adverse events is conjunctivitis and keratitis and the additional therapeutic agent is a preservative-free lubricating eye drop, an ocular vasoconstrictor, antibiotic, a steroid eye drop, or any combination thereof. In some embodiments, the one or more adverse events is conjunctivitis and the additional therapeutic agent is a preservative-free lubricating eye drop, an ocular vasoconstrictor, antibiotic, a steroid eye drop, or any combination thereof. In some embodiments, the one or more adverse events is keratitis and the additional therapeutic agent is a preservative-free lubricating eye drop, an ocular vasoconstrictor, antibiotic, a steroid eye drop, or any combination thereof. In some of any of the embodiments herein, the subject is administered a treatment with the additional therapeutic agent to eliminate or reduce the severity of the adverse event (e.g., conjunctivitis, conjunctival ulceration, and/or keratitis). In some embodiments, the treatment is eye cooling pads (e.g. THERA PEARL Eye Mask or similar). In some embodiments, the one or more adverse events is a recurrent infusion related reaction and the additional therapeutic agent is an antihistamine, acetaminophen and/or a corticosteroid. In some embodiments, the one or more adverse events is neutropenia and the additional therapeutic agent is growth factor support (G-CSF). In some embodiments, the one or more adverse events is hyperthyroidism and the additional agent is a non-selective beta-blockers (e.g., propranolol) or thionamides. In some embodiments, the one or more adverse events is hypothyroidism and the additional agent is a thyroid replacement hormone (e.g., levothyroxine or liothyroinine).

In one aspect, the subject treated with an anti-TF antibody-drug conjugates or antigen-binding fragments thereof described herein and an anti-PD-1 antibody or antigen-binding fragment thereof described herein is at risk of developing one or more adverse events. In some embodiments, the subject is administered an additional therapeutic agent to prevent the development of the adverse event or to reduce the severity of the adverse event. In some embodiments, the one or more adverse events the subject is at risk of developing is anemia, abdominal pain, hemorrhage, hyperthyroidism, hypothyroidism, hypokalemia, hyponatremia, epistaxis, fatigue, nausea, alopecia, conjunctivitis, keratitis, conjunctival ulceration, constipation, decreased appetite, diarrhea, vomiting, peripheral neuropathy, or general physical health deterioration, or any combination thereof. In some embodiments, the one or more adverse events is a grade 1 or greater adverse event. In some embodiments, the one or more adverse events is a grade 2 or greater adverse event. In some embodiments, the one or more adverse events is a grade 3 or greater adverse event. In some embodiments, the one or more adverse events is a grade 1 adverse event. In some embodiments, the one or more adverse events is a grade 2 adverse event. In some embodiments, the one or more adverse events is a grade 3 adverse event. In some embodiments, the one or more adverse events is a grade 4 adverse event. In some embodiments, the one or more adverse events is a serious adverse event. In some embodiments, the one or more adverse events is conjunctivitis, conjunctival ulceration, and/or keratitis and the additional agent is a preservative-free lubricating eye drop, an ocular vasoconstrictor, antibiotic, a steroid eye drop, or any combination thereof. In some embodiments, the one or more adverse events is conjunctivitis and keratitis and the additional agent is a preservative-free lubricating eye drop, an ocular vasoconstrictor, antibiotic, a steroid eye drop, or any combination thereof. In some embodiments, the one or more adverse events is conjunctivitis and the additional agent is a preservative-free lubricating eye drop, an ocular vasoconstrictor, antibiotic, a steroid eye drop, or any combination thereof. In some embodiments, the one or more adverse events is keratitis and the additional agent is a preservative-free lubricating eye drop, an ocular vasoconstrictor, antibiotic, a steroid eye drop, or any combination thereof. In some of any of the embodiments herein, the subject is administered a treatment with the additional therapeutic agent to prevent the development of the adverse event or to reduce the severity of the adverse event (e.g., conjunctivitis, conjunctival ulceration, and/or keratitis). In some embodiments, the treatment is eye cooling pads (e.g. THERA PEARL Eye Mask or similar). In some embodiments, the one or more adverse events is a recurrent infusion related reaction and the additional agent is an antihistamine, acetaminophen and/or a corticosteroid. In some embodiments, the one or more adverse events is neutropenia and the additional agent is growth factor support (G-CSF). In some embodiments, the one or more adverse events is hyperthyroidism and the additional agent is a non-selective beta-blockers (e.g., propranolol) or thionamides. In some embodiments, the one or more adverse events is hypothyroidism and the additional agent is a thyroid replacement hormone (e.g., levothyroxine or liothyroinine).

### V. Compositions

In some aspects, also provided herein are compositions (e.g., pharmaceutical compositions and therapeutic formulations) comprising any of the anti-TF antibody-drug conjugates or antigen-binding fragments thereof described herein and/or the anti-PD-1 antibody or antigen-binding fragments thereof described herein.

Therapeutic formulations are prepared for storage by mixing the active ingredient having the desired degree of purity with optional pharmaceutically acceptable carriers, excipients or stabilizers (Remington: The Science and Practice of Pharmacy, 20th Ed., Lippincott Williams & Wiklins, Pub., Gennaro Ed., Philadelphia, Pa. 2000).

Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers can be used to control the pH in a range which optimizes the therapeutic effectiveness, especially if stability is pH dependent. Buffers can be present at concentrations ranging from about 50 mM to about 250 mM. Suitable buffering agents for use with the present invention include both organic and inorganic acids and salts thereof. For example, citrate, phosphate, succinate, tartrate, fumarate, gluconate, oxalate, lactate, acetate. Additionally, buffers may be comprised of histidine and trimethylamine salts such as Tris.

Preservatives can be added to prevent microbial growth, and are typically present in a range from about 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intramolecular interactions. Tonicity agents can be present in any amount between about 0.1% to about 25% by weight or between about 1% to about 5% by weight, taking into account the relative amounts of the other ingredients. In some embodiments, tonicity agents include polyhydric sugar alcohols, trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, a-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) can be present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml or about 0.07 mg/ml to about 0.2 mg/ml. In some embodiments, non-ionic surfactants are present in a range of about 0.001% to about 0.1% w/v or about 0.01% to about 0.1% w/v or about 0.01% to about 0.025% w/v.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

Formulations comprising an anti-TF antibody-conjugate described herein for use in methods of treatment provided herein are described in WO2015/075201. In some embodiments, an anti-TF antibody-drug conjugate described herein is in a formulation comprising the anti-TF antibody drug conjugate, histidine, sucrose, and D-mannitol, wherein the formulation has a pH of about 6.0. In some embodiments, an anti-TF antibody-drug conjugate described herein is in a formulation comprising the anti-TF antibody drug conjugate at a concentration of about 10 mg/ml, histidine at a concentration of about 30 mM, sucrose at a concentration of about 88 mM, D-mannitol at a concentration of about 165 mM, wherein the formulation has a pH of about 6.0. In some embodiments, an anti-TF antibody-drug conjugate described herein is in a formulation comprising the anti-TF antibody drug conjugate at a concentration of 10 mg/ml, histidine at a concentration of 30 mM, sucrose at a concentration of 88 mM, D-mannitol at a concentration of 165 mM, wherein the formulation has a pH of 6.0. In some embodiments, the formulation comprises tisotumab vedotin at a concentration of 10 mg/ml, histidine at a concentration of 30 mM, sucrose at a concentration of 88 mM, D-mannitol at a concentration of 165 mM, wherein the formulation has a pH of 6.0.

In some embodiments provided herein, a formulation comprising the anti-TF antibody-conjugate described herein does not comprise a surfactant (i.e., is free of surfactant).

In order for the formulations to be used for in vivo administration, they must be sterile. The formulation may be rendered sterile by filtration through sterile filtration membranes. The therapeutic compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended.

The invention provides compositions comprising a population of anti-TF antibody-drug conjugates or antigen-binding fragments thereof as described herein for use in a method of treating cervical cancer as described herein. In some aspects, provided herein are compositions comprising a population of antibody-drug conjugates, wherein the antibody-drug conjugates comprise a linker attached to MMAE (vcMMAE), wherein the antibody-drug conjugate has the following structure:

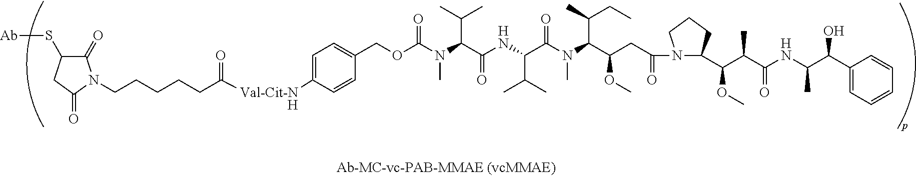

wherein p denotes a number from 1 to 8, e.g., 1, 2, 3, 4, 5, 6, 7 or 8, S represents a sulphydryl residue of the anti-TF antibody or antigen-binding fragment thereof, and Ab designates the anti-TF antibody or antigen-binding fragment thereof as described herein, such as tisotumab. In some embodiments, p denotes a number from 3 to 5. In some embodiments, the average value of p in the composition is about 4. In some embodiments, the population is a mixed population of antibody-drug conjugates in which p varies from 1 to 8 for each antibody-drug conjugate. In some embodiments, the population is a homogenous population of antibody-drug conjugates with each antibody-drug conjugate having the same value for p.

In some embodiments, a composition comprising an anti-TF antibody-drug conjugate or antigen-binding fragment thereof as described herein is coadministered with a composition comprising an anti-PD-1 antibody or antigen-binding fragment thereof as described herein. In some embodiments the coadministration is simultaneous or sequential. In some embodiments, the anti-TF antibody-drug conjugate as described herein is administered simultaneously with the anti-PD-1 antibody as described herein. In some embodiments, simultaneous means that the anti-TF antibody-drug conjugate described herein and the anti-PD-1 antibody described herein are administered to the subject less than about one hour apart, such as less than about 30 minutes apart, less than about 15 minutes apart, less than about 10 minutes apart or less than about 5 minutes apart. In some embodiments, simultaneous means that the anti-TF antibody-drug conjugate described herein and the anti-PD-1 antibody described herein are administered to the subject less than one hour apart, such as less than 30 minutes apart, less than 15 minutes apart, less than 10 minutes apart or less than 5 minutes apart. In some embodiments, the anti-TF antibody-drug conjugate described herein is administered sequentially with the anti-PD-1 antibody described herein. In some embodiments, sequential administration means that the anti-TF antibody-drug conjugate described herein and the anti-PD-1 antibody described herein are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart. In some embodiments, a composition comprising an ant-TF antibody-drug conjugate as described herein and/or an anti-PD-1 antibody as described herein is coadministered with one or more therapeutic agents to eliminate or reduce the severity of one or more adverse events. In some embodiments, a composition comprising an anti-TF antibody-drug conjugate as described herein and/or an anti-PD-1 antibody as described herein is coadministered with one or more therapeutic agents to prevent the development of the adverse event or to reduce the severity of the adverse event.

In some embodiments, a composition comprising an anti-TF antibody-drug conjugate as described herein and/or anti-PD-1 antibody as described herein is coadministered with one or additional therapeutic agents. In some embodiments the coadministration is simultaneous or sequential. In some embodiments, the anti-TF antibody-drug conjugate as described herein and/or anti-PD-1 antibody as described herein is administered simultaneously with the one or more additional therapeutic agents. In some embodiments, simultaneous means that the anti-TF antibody-drug conjugate described herein and/or anti-PD-1 antibody described herein and the one or more therapeutic agents are administered to the subject less than about one hour apart, such as less than about 30 minutes apart, less than about 15 minutes apart, less than about 10 minutes apart or less than about 5 minutes apart. In some embodiments, simultaneous means that the anti-TF antibody-drug conjugate described herein and/or anti-PD-1 antibody described herein and the one or more therapeutic agents are administered to the subject less than one hour apart, such as less than 30 minutes apart, less than 15 minutes apart, less than 10 minutes apart or less than 5 minutes apart. In some embodiments, the anti-TF antibody-drug conjugate described herein and/or anti-PD-1 antibody described herein is administered sequentially with the one or more additional therapeutic agents. In some embodiments, sequential administration means that the anti-TF antibody-drug conjugate described herein and/or anti-PD-1 antibody described herein and the one or more additional therapeutic agents are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart.

In some embodiments, a composition comprising an anti-TF antibody-drug conjugate as described herein and/or anti-PD-1 antibody as described herein is coadministered with one or more therapeutic agents to eliminate or reduce the severity of one or more adverse events. In some embodiments the coadministration is simultaneous or sequential. In some embodiments, the anti-TF antibody-drug conjugate described herein and/or anti-PD-1 antibody described herein is administered simultaneously with the one or more therapeutic agents to eliminate or reduce the severity of one or more adverse events. In some embodiments, simultaneous means that the anti-TF antibody-drug conjugate described herein and/or anti-PD-1 antibody described herein and the one or more therapeutic agents to eliminate or reduce the severity of one or more adverse events are administered to the subject less than about one hour apart, such as less than about 30 minutes apart, less than about 15 minutes apart, less than about 10 minutes apart or less than about 5 minutes apart. In some embodiments, simultaneous means that the anti-TF antibody-drug conjugate described herein and/or anti-PD-1 antibody described herein and the one or more therapeutic agents to eliminate or reduce the severity of one or more adverse events are administered to the subject less than one hour apart, such as less than 30 minutes apart, less than 15 minutes apart, less than 10 minutes apart or less than 5 minutes apart. In some embodiments, the anti-TF antibody-drug conjugate described herein and/or anti-PD-1 antibody described herein is administered sequentially with the one or more therapeutic agents to eliminate or reduce the severity of one or more adverse events. In some embodiments, sequential administration means that the anti-TF antibody-drug conjugate described herein and/or anti-PD-1 antibody described herein and the one or more additional therapeutic agents are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart. In some embodiments, the anti-TF antibody-drug conjugate described herein and/or anti-PD-1 antibody described herein is administered prior to the one or more therapeutic agents to eliminate or reduce the severity of one or more adverse events. In some embodiments, the one or more therapeutic agents to eliminate or reduce the severity of one or more adverse events is administered prior to the anti-TF antibody-drug conjugate described herein and/or anti-PD-1 antibody described herein.

### VI. Articles of Manufacture and Kits

In another aspect, an article of manufacture or kit is provided which comprises an anti-TF antibody-drug conjugate described herein and/or an anti-PD-1 antibody described herein. The article of manufacture or kit may further comprise instructions for use of the anti-TF antibody-drug conjugate described herein and/or anti-PD-1 antibody described herein in the methods of the invention. Thus, in certain embodiments, the article of manufacture or kit comprises instructions for the use of an anti-TF antibody-drug conjugate described herein and/or an anti-PD-1 antibody described herein in methods for treating cancer (e.g., breast cancer or cervical cancer) in a subject comprising administering to the subject an effective amount of an anti-TF antibody-drug conjugate described herein and/or anti-PD-1 antibody described herein. In some embodiments, the cancer is breast cancer. In some embodiments, the cancer is cervical cancer. In some embodiments, the cervical cancer is advanced stage cervical cancer. In some embodiments, the advanced stage cervical cancer is metastatic cervical cancer. In some embodiments, the advanced stage cervical cancer is a stage 3 or stage 4 cervical cancer. In some embodiments, the cervical cancer is metastatic cancer and recurrent cancer. In some embodiments the cervical cancer is recurrent cancer. In some embodiments, the subject is not a candidate for curative therapy. In some embodiments, the subject has not received prior systemic therapy for the cervical cancer. In some embodiments, the subject is a human.

The article of manufacture or kit may further comprise a container. Suitable containers include, for example, bottles, vials (e.g., dual chamber vials), syringes (such as single or dual chamber syringes) and test tubes. In some embodiments, the container is a vial. The container may be formed from a variety of materials such as glass or plastic. The container holds the formulation.

The article of manufacture or kit may further comprise a label or a package insert, which is on or associated with the container, may indicate directions for reconstitution and/or use of the formulation. The label or package insert may further indicate that the formulation is useful or intended for subcutaneous, intravenous (e.g., intravenous infusion), or other modes of administration for treating cancer in a subject such as breast cancer or cervical cancer described herein (e.g., advanced cervical cancer such as grade 3 or grade 4 or metastatic cervical cancer). The container holding the formulation may be a single-use vial or a multi-use vial, which allows for repeat administrations of the reconstituted formulation. The article of manufacture or kit may further comprise a second container comprising a suitable diluent. The article of manufacture or kit may further include other materials desirable from a commercial, therapeutic, and user standpoint, including other buffers, diluents, filters, needles, syringes, and package inserts with instructions for use.

The article of manufacture or kit herein optionally further comprises a container comprising a second medicament, wherein the anti-TF antibody-drug conjugate is a first medicament, and which article or kit further comprises instructions on the label or package insert for treating the subject with the second medicament, in an effective amount. In some embodiments, the second medicament is an anti-PD-1 antibody as described herein. In some embodiments, the label or package insert indicates that the first and second medicaments are to be administered sequentially or simultaneously, as described herein.

The article of manufacture or kit herein optionally further comprises a container comprising a third medicament, wherein the third medicament is for eliminating or reducing the severity of one or more adverse events, wherein the anti-TF antibody-drug conjugate described herein is a first medicament, the anti-PD-1 antibody described herein is a second medicament, and which article or kit further comprises instructions on the label or package insert for treating the subject with the third medicament, in an effective amount. In some embodiments, the label or package insert indicates that the first, second and third medicaments are to be administered sequentially or simultaneously, as described herein, for example wherein the label or package insert indicates that the anti-TF antibody-drug conjugate described herein is to be administered first, followed by administration of the anti-PD-1 antibody described herein, followed by administration of the third medicament.

In some embodiments, the anti-TF antibody-drug conjugate described herein and/or anti-PD-1 antibody described herein is present in the container as a lyophilized powder. In some embodiments, the lyophilized powder is in a hermetically sealed container, such as a vial, an ampoule or sachette, indicating the quantity of the active agent. Where the pharmaceutical is administered by injection, an ampoule of sterile water for injection or saline can be, for example, provided, optionally as part of the kit, so that the ingredients can be mixed prior to administration. Such kits can further include, if desired, one or more of various conventional pharmaceutical components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components can also be included in the kit.

### VII. Exemplary Embodiments

Among the embodiments provided herein are:
1. A method of treating cancer in a subject, the method comprising administering to the subject an anti-PD-1 antibody or an antigen-binding fragment thereof, wherein the antibody binds to Programmed Death-1 (PD-1) and inhibits PD-1 activity, wherein the anti-PD-1 antibody or antigen-binding fragment thereof comprises the complementary determining regions (CDRs) of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof, and an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof.
2. The method of embodiment 1, wherein the anti-PD-1 antibody or antigen-binding fragment thereof comprises the CDRs of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.
3. The method of embodiment 1 or 2, wherein the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof.
4. The method of embodiment 1 or 2, wherein the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.
5. The method of embodiment 1, wherein the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof.
6. The method of embodiment 1, wherein the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.
7. The method of any one of embodiments 1-6, wherein the antibody-drug conjugate is administered at a dose ranging from about 0.9 mg/kg to about 2.1 mg/kg.
8. The method of embodiment 7, wherein the antibody-drug conjugate is administered at a dose of about 2.0 mg/kg.
9. The method of embodiment 7, wherein the antibody-drug conjugate is administered at a dose of 2.0 mg/kg.
10. The method of any one of embodiments 1-9, wherein the antibody-drug conjugate is administered once about every 1 week, once about every 2 weeks, once about every 3 weeks or once about every 4 weeks.
11. The method of embodiment 10, wherein the antibody-drug conjugate is administered once about every 3 weeks.
12. The method of any one of embodiments 1-11, wherein the anti-PD-1 antibody or antigen-binding fragment thereof comprises a heavy chain variable region and a light chain variable region, wherein the heavy chain variable region comprises:
(i) a CDR-H1 comprising the amino acid sequence of SEQ ID NO:17;
(ii) a CDR-H2 comprising the amino acid sequence of SEQ ID NO:18; and
   (iii) a CDR-H3 comprising the amino acid sequence of SEQ ID NO:19; and wherein the light chain variable region comprises:
   (i) a CDR-L1 comprising the amino acid sequence of SEQ ID NO:20;
   (ii) a CDR-L2 comprising the amino acid sequence of SEQ ID NO:21; and
   (iii) a CDR-L3 comprising the amino acid sequence of SEQ ID NO:22.
13. The method of any one of embodiments 1-12, wherein the anti-PD-1 antibody or antigen-binding fragment thereof comprises a heavy chain variable region comprising an amino acid sequence having at least 85% sequence identity to the amino acid sequence of SEQ ID NO:31 and a light chain variable region comprising an amino acid sequence having at least 85% sequence identity to the amino acid sequence of SEQ ID NO:32.
14. The method of any one of embodiments 1-13, wherein the anti-PD-1 antibody or antigen-binding fragment thereof comprises a heavy chain variable region comprising the amino acid sequence of SEQ ID NO:31 and a light chain variable region comprising the amino acid sequence of SEQ ID NO:32.
15. The method of any one of embodiments 1-14, wherein the anti-PD-1 antibody or antigen-binding fragment thereof is nivolumab.
16. The method of any one of embodiments 1-15, wherein the anti-PD-1 antibody or antigen-binding fragment thereof is administered at a dose ranging from about 0.5 mg/kg to about 4.1 mg/kg.
17. The method of any one of embodiments 1-15, wherein the anti-PD-1 antibody or antigen-binding fragment thereof is administered at a flat dose ranging from about 50 mg to about 500 mg.
18. The method of any one of embodiments 1-15, wherein the anti-PD-1 antibody or antigen-binding fragment thereof is administered at a flat dose of about 240 mg.
19. The method of any one of embodiments 1-15, wherein the anti-PD-1 antibody or antigen-binding fragment thereof is administered at a flat dose of about 480 mg.
20. The method of any one of embodiments 1-19, wherein the anti-PD-1 antibody or antigen-binding fragment thereof is administered once about every 1 week, once about every 2 weeks, once about every 3 weeks or once about every 4 weeks.
21. The method of embodiment 20, wherein the anti-PD-1 antibody or antigen-binding fragment thereof is administered once about every 2 weeks.
22. The method of any one of embodiments 1-21, wherein the cancer is breast cancer.
23. The method of any one of embodiments 1-21, wherein the cancer is cervical cancer.
24. The method of embodiment 23, wherein the subject is not a candidate for curative therapy.
25. The method of embodiment 24, wherein curative therapy comprises radiotherapy and/or exenterative surgery.
26. The method of embodiment 23, wherein the subject has not received prior systemic therapy for the cervical cancer.
27. The method of any one of embodiment 23-26, wherein the cervical cancer is an adenocarcinoma, an adenosquamous carcinoma or a squamous cell carcinoma.
28. The method of any one of embodiments 23-27, wherein the cervical cancer is an advanced stage cervical cancer.
29. The method of embodiment 28, wherein the advanced stage cervical cancer is a stage 3 or stage 4 cervical cancer.
30. The method of embodiment 28 or 29, wherein the advanced stage cervical cancer is metastatic cervical cancer.
31. The method of any one of embodiments 23-30, wherein the cervical cancer is recurrent cervical cancer.
32. The method of any one of embodiments 1-31, wherein the monomethyl auristatin is monomethyl auristatin E (MMAE).
33. The method of any one of embodiments 1-32, wherein the anti-TF antibody or antigen-binding fragment thereof of the antibody-drug conjugate is a monoclonal antibody or a monoclonal antigen-binding fragment thereof.
34. The method of any one of embodiments 1-33, wherein the anti-TF antibody or antigen-binding fragment thereof of the antibody-drug conjugate comprises a heavy chain variable region and a light chain variable region, wherein the heavy chain variable region comprises:
   (i) a CDR-H1 comprising the amino acid sequence of SEQ ID NO:1;
   (ii) a CDR-H2 comprising the amino acid sequence of SEQ ID NO:2; and
   (iii) a CDR-H3 comprising the amino acid sequence of SEQ ID NO:3; and wherein the light chain variable region comprises:
   (i) a CDR-L1 comprising the amino acid sequence of SEQ ID NO:4;
   (ii) a CDR-L2 comprising the amino acid sequence of SEQ ID NO:5; and
   (iii) a CDR-L3 comprising the amino acid sequence of SEQ ID NO:6.
35. The method of any one of embodiments 1-34, wherein the anti-TF antibody or antigen-binding fragment thereof of the antibody-drug conjugate comprises a heavy chain variable region comprising an amino acid sequence having at least 85% sequence identity to the amino acid sequence of SEQ ID NO:7 and a light chain variable region comprising an amino acid sequence having at least 85% sequence identity to the amino acid sequence of SEQ ID NO:8.
36. The method of any one of embodiments 1-35, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.
37. The method of any one of embodiments 1-36, wherein the anti-TF antibody of the antibody-drug conjugate is tisotumab.
38. The method of any one of embodiments 1-37, wherein the antibody-drug conjugate further comprises a linker between the anti-TF antibody or antigen-binding fragment thereof and the monomethyl auristatin.
39. The method of embodiment 38, wherein the linker is a cleavable peptide linker.
40. The method of embodiment 39, wherein the cleavable peptide linker has a formula: -MC-vc-PAB-, wherein:
   a) MC is:

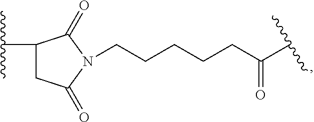 [SMILES: *C(=O)CCCCCN1C(=O)CC(*)C1=O]

b) vc is the dipeptide valine-citrulline, and
c) PAB is:

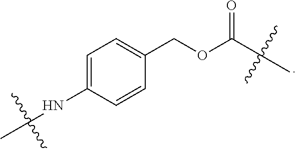 [SMILES: *Nc1ccc(COC(*)=O)cc1]

41. The method of any one of embodiments 38-40, wherein the linker is attached to sulphydryl residues of the anti-TF antibody obtained by partial reduction or full reduction of the anti-TF antibody or antigen-binding fragment thereof.
42. The method of embodiment 41, wherein the linker is attached to MMAE (vcMMAE), wherein the antibody-drug conjugate has the following structure:

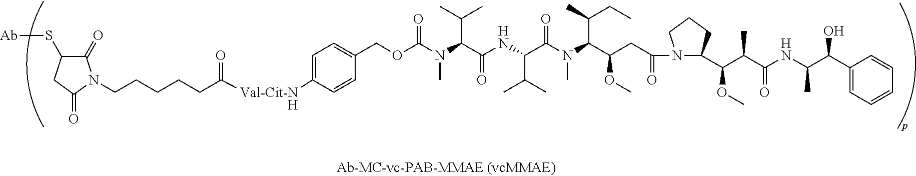

wherein p denotes a number from 1 to 8, S represents a sulphydryl residue of the antd-TF antibody, and Ab designates the anti-TF antibody or antigen-binding fragment thereof.
43. The method of embodiment 42, wherein the average value ofp in a population ofthe antibody-drug conjugates is about 4.
44. The method of any one of embodiments 1-43, wherein the antibody-drug conjugate is tisotumab vedotin.
45. The method of any one of embodiments 1-44, wherein the route of administration for the antibody-drug conjugate is intravenous.
46. The method of any one of embodiments 1-45, wherein the route of administration for the anti-PD-1 antibody or antigen-binding fragment thereof is intravenous.
47. The method of any one of embodiments 1-46, wherein the anti-PD-1 antibody or antigen-binding fragment thereof and the antibody-drug conjugate are administered sequentially.
48. The method of any one of embodiments 1-46, wherein the anti-PD-1 antibody or antigen-binding fragment thereof and the antibody-drug conjugate are administered simultaneously.
49. The method of any one of embodiments 1-48, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express TF.
50. The method of any one of embodiments 1-49, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express PD-L1.
51. The method of any one of embodiments 1-50, wherein a tumor derived from the cancer comprises one or more cells that express PD-L1, PD-L2, or both PD-L1 and PD-L2.
52. The method of any one of embodiments 1-51, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express PD-1.
53. The method of any one of embodiments 1-52, wherein one or more therapeutic effects in the subject is improved after administration of the antibody-drug conjugate and the anti-PD-1 antibody or antigen-binding fragment thereof relative to a baseline.
54. The method of embodiment 53, wherein the one or more therapeutic effects is selected from the group consisting of: size of a tumor derived from the cancer, objective response rate, duration of response, time to response, progression free survival, and overall survival.
55. The method of any one of embodiments 1-54, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody-drug conjugate and the anti-PD-1 antibody or antigen-binding fragment thereof.
56. The method of any one of embodiments 1-55, wherein the objective response rate is at least about 20%, at least about 25%, at least about 30%, at least about 35%, at least about 40%, at least about 45%, at least about 50%, at least about 60%, at least about 70%, or at least about 80%.
57. The method of any one of embodiments 1-56, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
58. The method of any one of embodiments 1-57, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
59. The method of any one of embodiments 1-58, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
60. The method of any one of embodiments 1-59, wherein the subject has one or more adverse events and is further administered an additional therapeutic agent to eliminate or reduce the severity of the one or more adverse events.
61. The method of any one of embodiments 1-60, wherein the subject is at risk of developing one or more adverse events and is further administered an additional therapeutic agent to prevent or reduce the severity of the one or more adverse events.
62. The method of embodiment 60 or embodiment 61, wherein the one or more adverse events is anemia, abdominal pain, hemorrhage, hyperthyroidism, hypothyroidism, hypokalemia, hyponatremia, epistaxis, fatigue, nausea, alopecia, conjunctivitis, keratitis, conjunctival ulceration, constipation, decreased appetite, diarrhea, vomiting, peripheral neuropathy, or general physical health deterioration.
63. The method of any one of embodiments 60-62, wherein the one or more adverse events is a grade 3 or greater adverse event.
64. The method of any one of embodiments 60-62, wherein the one or more adverse events is a serious adverse event.
65. The method of embodiment 60 or embodiment 61, wherein the one or more adverse events is conjunctivitis, conjunctival ulceration, and/or keratitis and the additional agent is a preservative-free lubricating eye drop, an ocular vasoconstrictor, antibiotic, and/or a steroid eye drop.
66. The method of any one of embodiments 1-65, wherein the subject is a human.
67. The method of any one of embodiments 1-66, wherein the antibody-drug conjugate is in a pharmaceutical composition comprising the antibody-drug conjugate and a pharmaceutical acceptable carrier.
68. The method of any one of embodiments 1-67, wherein the anti-PD-1 antibody or antigen-binding fragment thereof is in a pharmaceutical composition comprising the anti-PD-1 antibody or antigen-binding fragment thereof and a pharmaceutical acceptable carrier.
69. An antibody-drug conjugate that binds to TF for use in the treatment of cancer wherein the antibody-drug conjugate is for administration, or to be administered in combination with an anti-PD-1 antibody or an antigen-binding fragment thereof, wherein the antibody-drug conjugate comprises an anti-TF antibody or an antigen-binding fragment thereof conjugated to a monomethyl auristatin or a functional analog thereof or a functional derivative thereof, wherein the anti-PD-1 antibody or the antigen-binding fragment thereof inhibits PD-1 activity, and wherein the anti-PD-1 antibody or antigen-binding fragment thereof comprises the complementary determining regions (CDRs) of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof.
70. The antibody-drug conjugate for use of embodiment 69, wherein the anti-PD-1 antibody or antigen-binding fragment thereof comprises the CDRs of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.
71. The antibody-drug conjugate for use of embodiment 69 or 70, wherein the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof.
72. The antibody-drug conjugate for use of embodiment 69 or 70, wherein the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.
73. The antibody-drug conjugate for use of embodiment 69, wherein the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof.
74. The antibody-drug conjugate for use of embodiment 69, wherein the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.
75. The antibody-drug conjugate for use of any one of embodiments 69-74, wherein the antibody-drug conjugate is administered at a dose ranging from about 0.9 mg/kg to about 2.1 mg/kg.
76. The antibody-drug conjugate for use of embodiment 75, wherein the antibody-drug conjugate is administered at a dose of about 2.0 mg/kg.
77. The antibody-drug conjugate for use of embodiment 75, wherein the antibody-drug conjugate is administered at a dose of 2.0 mg/kg.
78. The antibody-drug conjugate for use of any one of embodiments 69-77, wherein the antibody-drug conjugate is administered once about every 1 week, once about every 2 weeks, once about every 3 weeks or once about every 4 weeks.
79. The antibody-drug conjugate for use of embodiment 78, wherein the antibody-drug conjugate is administered once about every 3 weeks.
80. The antibody-drug conjugate for use of any one of embodiments 69-79, wherein the anti-PD-1 antibody or antigen-binding fragment thereof comprises a heavy chain variable region and a light chain variable region, wherein the heavy chain variable region comprises:
   (i) a CDR-H1 comprising the amino acid sequence of SEQ ID NO:17;
   (ii) a CDR-H2 comprising the amino acid sequence of SEQ ID NO:18; and
   (iii) a CDR-H3 comprising the amino acid sequence of SEQ ID NO:19; and wherein the light chain variable region comprises:
   (i) a CDR-L1 comprising the amino acid sequence of SEQ ID NO:20;
   (ii) a CDR-L2 comprising the amino acid sequence of SEQ ID NO:21; and
   (iii) a CDR-L3 comprising the amino acid sequence of SEQ ID NO:22.
81. The antibody-drug conjugate for use of any one of embodiments 69-80, wherein the anti-PD-1 antibody or antigen-binding fragment thereof comprises a heavy chain variable region comprising an amino acid sequence having at least 85% sequence identity to the amino acid sequence of SEQ ID NO:31 and a light chain variable region comprising an amino acid sequence having at least 85% sequence identity to the amino acid sequence of SEQ ID NO:32.
82. The antibody-drug conjugate for use of any one of embodiments 69-81, wherein the anti-PD-1 antibody or antigen-binding fragment thereof comprises a heavy chain variable region comprising the amino acid sequence of SEQ ID NO:31 and a light chain variable region comprising the amino acid sequence of SEQ ID NO:32.
83. The antibody-drug conjugate for use of any one of embodiments 69-82, wherein the anti-PD-1 antibody or antigen-binding fragment thereof is nivolumab.
84. The antibody-drug conjugate for use of any one of embodiments 69-83, wherein the anti-PD-1 antibody or antigen-binding fragment thereof is administered at a dose ranging from about 0.5 mg/kg to about 4.1 mg/kg.
85. The antibody-drug conjugate for use of any one of embodiments 69-83, wherein the anti-PD-1 antibody or antigen-binding fragment thereof is administered at a flat dose ranging from about 50 mg to about 500 mg.
86. The antibody-drug conjugate for use of any one of embodiments 69-83, wherein the anti-PD-1 antibody or antigen-binding fragment thereof is administered at a flat dose of about 240 mg.
87. The antibody-drug conjugate for use of any one of embodiments 69-83, wherein the anti-PD-1 antibody or antigen-binding fragment thereof is administered at a flat dose of about 480 mg.
88. The antibody-drug conjugate for use of any one of embodiments 69-87, wherein the anti-PD-1 antibody or antigen-binding fragment thereof is administered once about every 1 week, once about every 2 weeks, once about every 3 weeks or once about every 4 weeks.
89. The antibody-drug conjugate for use of embodiment 88, wherein the anti-PD-1 antibody or antigen-binding fragment thereof is administered once about every 2 weeks.
90. The antibody-drug conjugate for use of any one of embodiments 69-89, wherein the cancer is breast cancer.
91. The antibody-drug conjugate for use of any one of embodiments 69-89, wherein the cancer is cervical cancer.
92. The antibody-drug conjugate for use of embodiment 91, wherein the subject is not a candidate for curative therapy.
93. The antibody-drug conjugate for use of embodiment 92, wherein curative therapy comprises radiotherapy and/or exenterative surgery.
94. The antibody-drug conjugate for use of embodiment 91, wherein the subject has not received prior systemic therapy for the cervical cancer.
95. The antibody-drug conjugate for use of any one of embodiment 91-94, wherein the cervical cancer is an adenocarcinoma, an adenosquamous carcinoma or a squamous cell carcinoma.
96. The antibody-drug conjugate for use of any one of embodiments 91-95, wherein the cervical cancer is an advanced stage cervical cancer.
97. The antibody-drug conjugate for use of embodiment 96, wherein the advanced stage cervical cancer is a stage 3 or stage 4 cervical cancer.
98. The antibody-drug conjugate for use of embodiment 96 or 97, wherein the advanced stage cervical cancer is metastatic cervical cancer.
99. The antibody-drug conjugate for use of any one of embodiments 91-98, wherein the cervical cancer is recurrent cervical cancer.
100. The antibody-drug conjugate for use of any one of embodiments 69-99, wherein the monomethyl auristatin is monomethyl auristatin E (MMAE).
101. The antibody-drug conjugate for use of any one of embodiments 69-100, wherein the anti-TF antibody or antigen-binding fragment thereof of the antibody-drug conjugate is a monoclonal antibody or a monoclonal antigen-binding fragment thereof.
102. The antibody-drug conjugate for use of any one of embodiments 69-101, wherein the anti-TF antibody or antigen-binding fragment thereof of the antibody-drug conjugate comprises a heavy chain variable region and a light chain variable region, wherein the heavy chain variable region comprises:
   (i) a CDR-H1 comprising the amino acid sequence of SEQ ID NO:1;
   (ii) a CDR-H2 comprising the amino acid sequence of SEQ ID NO:2; and
   (iii) a CDR-H3 comprising the amino acid sequence of SEQ ID NO:3; and wherein the light chain variable region comprises:
   (i) a CDR-L1 comprising the amino acid sequence of SEQ ID NO:4;
   (ii) a CDR-L2 comprising the amino acid sequence of SEQ ID NO:5; and
   (iii) a CDR-L3 comprising the amino acid sequence of SEQ ID NO:6.
103. The antibody-drug conjugate for use of any one of embodiments 69-102, wherein the anti-TF antibody or antigen-binding fragment thereof of the antibody-drug conjugate comprises a heavy chain variable region comprising an amino acid sequence having at least 85% sequence identity to the amino acid sequence of SEQ ID NO:7 and a light chain variable region comprising an amino acid sequence having at least 85% sequence identity to the amino acid sequence of SEQ ID NO:8.
104. The antibody-drug conjugate for use of any one of embodiments 69-103, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.
105. The antibody-drug conjugate for use of any one of embodiments 69-104, wherein the anti-TF antibody of the antibody-drug conjugate is tisotumab.
106. The antibody-drug conjugate for use of any one of embodiments 69-105, wherein the antibody-drug conjugate further comprises a linker between the anti-TF antibody or antigen-binding fragment thereof and the monomethyl auristatin.
107. The antibody-drug conjugate for use of embodiment 106, wherein the linker is a cleavable peptide linker.
108. The antibody-drug conjugate for use of embodiment 107, wherein the cleavable peptide linker has a formula: -MC-vc-PAB-, wherein:
   a) MC is:

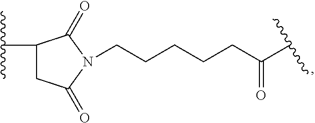 [SMILES: *C(=O)CCCCCN1C(=O)CC(*)C1=O]

b) vc is the dipeptide valine-citrulline, and
c) PAB is:

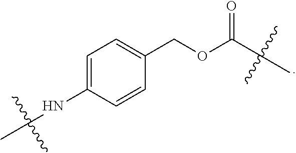 [SMILES: *Nc1ccc(COC(*)=O)cc1]

109. The antibody-drug conjugate for use of any one of embodiments 106-108, wherein the linker is attached to sulphydryl residues of the anti-TF antibody obtained by partial reduction or full reduction of the anti-TF antibody or antigen-binding fragment thereof.
110. The antibody-drug conjugate for use of embodiment 109, wherein the linker is attached to MMAE (vcMMAE), wherein the antibody-drug conjugate has the following structure:

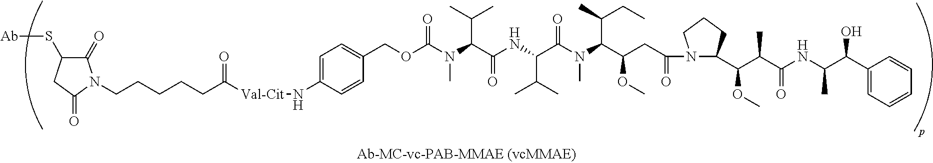

wherein p denotes a number from 1 to 8, S represents a sulphydryl residue of the anti-TF antibody, and Ab designates the anti-TF antibody or antigen-binding fragment thereof.
111. The antibody-drug conjugate for use of embodiment 110, wherein the average value of p in a population of the antibody-drug conjugates is about 4.
112. The antibody-drug conjugate for use of any one of embodiments 69-111, wherein the antibody-drug conjugate is tisotumab vedotin.
113. The antibody-drug conjugate for use of any one of embodiments 69-112, wherein the route of administration for the antibody-drug conjugate is intravenous.
114. The antibody-drug conjugate for use of any one of embodiments 69-113, wherein the route of administration for the anti-PD-1 antibody or antigen-binding fragment thereof is intravenous.
115. The antibody-drug conjugate for use of any one of embodiments 69-114, wherein the anti-PD-1 antibody or antigen-binding fragment thereof and the antibody-drug conjugate are administered sequentially.
116. The antibody-drug conjugate for use of any one of embodiments 69-114, wherein the anti-PD-1 antibody or antigen-binding fragment thereof and the antibody-drug conjugate are administered simultaneously.
117. The antibody-drug conjugate for use of any one of embodiments 69-116, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express TF.
118. The antibody-drug conjugate for use of any one of embodiments 69-117, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express PD-L1.
119. The antibody-drug conjugate for use of any one of embodiments 69-118, wherein a tumor derived from the cancer comprises one or more cells that express PD-L1, PD-L2, or both PD-L1 and PD-L2.
120. The antibody-drug conjugate for use of any one of embodiments 69-119, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express PD-1.
121. The antibody-drug conjugate for use of any one of embodiments 69-120, wherein one or more therapeutic effects in the subject is improved after administration of the antibody-drug conjugate and the anti-PD-1 antibody or antigen-binding fragment thereof relative to a baseline.
122. The antibody-drug conjugate for use of embodiment 121, wherein the one or more therapeutic effects is selected from the group consisting of: size of a tumor derived from the cancer, objective response rate, duration of response, time to response, progression free survival, and overall survival.
123. The antibody-drug conjugate for use of any one of embodiments 69-122, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody-drug conjugate and the anti-PD-1 antibody or antigen-binding fragment thereof.
124. The antibody-drug conjugate for use of any one of embodiments 69-123, wherein the objective response rate is at least about 20%, at least about 25%, at least about 30%, at least about 35%, at least about 40%, at least about 45%, at least about 50%, at least about 60%, at least about 70%, or at least about 80%.
125. The antibody-drug conjugate for use of any one of embodiments 69-124, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
126. The antibody-drug conjugate for use of any one of embodiments 69-125, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
127. The antibody-drug conjugate for use of any one of embodiments 69-126, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
128. The antibody-drug conjugate for use of any one of embodiments 69-127, wherein the subject has one or more adverse events and is further administered an additional therapeutic agent to eliminate or reduce the severity of the one or more adverse events.
129. The antibody-drug conjugate for use of any one of embodiments 69-128, wherein the subject is at risk of developing one or more adverse events and is further administered an additional therapeutic agent to prevent or reduce the severity of the one or more adverse events.
130. The antibody-drug conjugate for use of embodiment 128 or embodiment 129, wherein the one or more adverse events is anemia, abdominal pain, hemorrhage, hyperthyroidism, hypothyroidism, hypokalemia, hyponatremia, epistaxis, fatigue, nausea, alopecia, conjunctivitis, keratitis, conjunctival ulceration, constipation, decreased appetite, diarrhea, vomiting, peripheral neuropathy, or general physical health deterioration.
131. The antibody-drug conjugate for use of any one of embodiments 128-130, wherein the one or more adverse events is a grade 3 or greater adverse event.
132. The antibody-drug conjugate for use of any one of embodiments 128-130, wherein the one or more adverse events is a serious adverse event.
133. The antibody-drug conjugate for use of embodiment 128 or embodiment 129, wherein the one or more adverse events is conjunctivitis, conjunctival ulceration, and/or keratitis and the additional agent is a preservative-free lubricating eye drop, an ocular vasoconstrictor, antibiotic, and/or a steroid eye drop.
134. The antibody-drug conjugate for use of any one of embodiments 69-133, wherein the subject is a human.
135. The antibody-drug conjugate for use of any one of embodiments 69-134, wherein the antibody-drug conjugate is in a pharmaceutical composition comprising the antibody-drug conjugate and a pharmaceutical acceptable carrier.
136. The antibody-drug conjugate for use of any one of embodiments 69-135, wherein the anti-PD-1 antibody or antigen-binding fragment thereof is in a pharmaceutical composition comprising the anti-PD-1 antibody or antigen-binding fragment thereof and a pharmaceutical acceptable carrier.
137. Use of an antibody-drug conjugate that binds to TF for the manufacture of a medicament for treating cancer in a subject, wherein the medicament is for use in combination with an anti-PD-1 antibody or an antigen-binding fragment thereof, wherein the antibody-drug conjugate comprises an anti-TF antibody or an antigen-binding fragment thereof conjugated to a monomethyl auristatin or a functional analog thereof or a functional derivative thereof, wherein the anti-PD-1 antibody or the antigen-binding fragment thereof inhibits PD-1 activity, and wherein the anti-PD-1 antibody or antigen-binding fragment thereof comprises the complementary determining regions (CDRs) of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof.
138. The use of embodiment 137, wherein the anti-PD-1 antibody or antigen-binding fragment thereof comprises the CDRs of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.
139. The use of embodiment 137 or 138, wherein the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof.
140. The use of embodiment 137 or 138, wherein the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.
141. The use of embodiment 137, wherein the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof.
142. The use of embodiment 137, wherein the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.
143. The use of any one of embodiments 137-142, wherein the antibody-drug conjugate is administered at a dose ranging from about 0.9 mg/kg to about 2.1 mg/kg.
144. The use of embodiment 143, wherein the antibody-drug conjugate is administered at a dose of about 2.0 mg/kg.
145. The use of embodiment 143, wherein the antibody-drug conjugate is administered at a dose of 2.0 mg/kg.
146. The use of any one of embodiments 137-145, wherein the antibody-drug conjugate is administered once about every 1 week, once about every 2 weeks, once about every 3 weeks or once about every 4 weeks.
147. The use of embodiment 146, wherein the antibody-drug conjugate is administered once about every 3 weeks.
148. The use of any one of embodiments 137-147, wherein the anti-PD-1 antibody or antigen-binding fragment thereof comprises a heavy chain variable region and a light chain variable region, wherein the heavy chain variable region comprises:
   (i) a CDR-H1 comprising the amino acid sequence of SEQ ID NO:17;
   (ii) a CDR-H2 comprising the amino acid sequence of SEQ ID NO:18; and
   (iii) a CDR-H3 comprising the amino acid sequence of SEQ ID NO:19; and wherein the light chain variable region comprises:
   (i) a CDR-L1 comprising the amino acid sequence of SEQ ID NO:20;
   (ii) a CDR-L2 comprising the amino acid sequence of SEQ ID NO:21; and
   (iii) a CDR-L3 comprising the amino acid sequence of SEQ ID NO:22.
149. The use of any one of embodiments 137-148, wherein the anti-PD-1 antibody or antigen-binding fragment thereof comprises a heavy chain variable region comprising an amino acid sequence having at least 85% sequence identity to the amino acid sequence of SEQ ID NO:31 and a light chain variable region comprising an amino acid sequence having at least 85% sequence identity to the amino acid sequence of SEQ ID NO:32.
150. The use of any one of embodiments 137-149, wherein the anti-PD-1 antibody or antigen-binding fragment thereof comprises a heavy chain variable region comprising the amino acid sequence of SEQ ID NO:31 and a light chain variable region comprising the amino acid sequence of SEQ ID NO:32.
151. The use of any one of embodiments 137-150, wherein the anti-PD-1 antibody or antigen-binding fragment thereof is nivolumab.
152. The use of any one of embodiments 137-151, wherein the anti-PD-1 antibody or antigen-binding fragment thereof is administered at a dose ranging from about 0.5 mg/kg to about 4.1 mg/kg.
153. The use of any one of embodiments 137-151, wherein the anti-PD-1 antibody or antigen-binding fragment thereof is administered at a flat dose ranging from about 50 mg to about 500 mg.
154. The use of any one of embodiments 137-151, wherein the anti-PD-1 antibody or antigen-binding fragment thereof is administered at a flat dose of about 240 mg.
155. The use of any one of embodiments 137-151, wherein the anti-PD-1 antibody or antigen-binding fragment thereof is administered at a flat dose of about 480 mg.
156. The use of any one of embodiments 137-155, wherein the anti-PD-1 antibody or antigen-binding fragment thereof is administered once about every 1 week, once about every 2 weeks, once about every 3 weeks or once about every 4 weeks.
157. The use of embodiment 156, wherein the anti-PD-1 antibody or antigen-binding fragment thereof is administered once about every 2 weeks.
158. The use of any one of embodiments 137-157, wherein the cancer is breast cancer.
159. The use of any one of embodiments 137-157, wherein the cancer is cervical cancer.
160. The use of embodiment 159, wherein the subject is not a candidate for curative therapy.
161. The use of embodiment 160, wherein curative therapy comprises radiotherapy and/or exenterative surgery.
162. The use of embodiment 159, wherein the subject has not received prior systemic therapy for the cervical cancer.
163. The use of any one of embodiment 159-162, wherein the cervical cancer is an adenocarcinoma, an adenosquamous carcinoma or a squamous cell carcinoma.
164. The use of any one of embodiments 159-163, wherein the cervical cancer is an advanced stage cervical cancer.
165. The use of embodiment 164, wherein the advanced stage cervical cancer is a stage 3 or stage 4 cervical cancer.
166. The use of embodiment 164 or 165, wherein the advanced stage cervical cancer is metastatic cervical cancer.
167. The use of any one of embodiments 159-166, wherein the cervical cancer is recurrent cervical cancer.
168. The use of any one of embodiments 137-167, wherein the monomethyl auristatin is monomethyl auristatin E (MMAE).
169. The use of any one of embodiments 137-168, wherein the anti-TF antibody or antigen-binding fragment thereof of the antibody-drug conjugate is a monoclonal antibody or a monoclonal antigen-binding fragment thereof.
170. The use of any one of embodiments 137-169, wherein the anti-TF antibody or antigen-binding fragment thereof of the antibody-drug conjugate comprises a heavy chain variable region and a light chain variable region, wherein the heavy chain variable region comprises:
   (i) a CDR-H1 comprising the amino acid sequence of SEQ ID NO:1;
   (ii) a CDR-H2 comprising the amino acid sequence of SEQ ID NO:2; and
   (iii) a CDR-H3 comprising the amino acid sequence of SEQ ID NO:3; and wherein the light chain variable region comprises:
   (i) a CDR-L1 comprising the amino acid sequence of SEQ ID NO:4;
   (ii) a CDR-L2 comprising the amino acid sequence of SEQ ID NO:5; and
   (iii) a CDR-L3 comprising the amino acid sequence of SEQ ID NO:6.
171. The use of any one of embodiments 137-170, wherein the anti-TF antibody or antigen-binding fragment thereof of the antibody-drug conjugate comprises a heavy chain variable region comprising an amino acid sequence having at least 85% sequence identity to the amino acid sequence of SEQ ID NO:7 and a light chain variable region comprising an amino acid sequence having at least 85% sequence identity to the amino acid sequence of SEQ ID NO:8.
172. The use of any one of embodiments 137-171, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.
173. The use of any one of embodiments 137-172, wherein the anti-TF antibody of the antibody-drug conjugate is tisotumab.
174. The use of any one of embodiments 137-173, wherein the antibody-drug conjugate further comprises a linker between the anti-TF antibody or antigen-binding fragment thereof and the monomethyl auristatin.
175. The use of embodiment 174, wherein the linker is a cleavable peptide linker.
176. The use of embodiment 175, wherein the cleavable peptide linker has a formula: -MC-vc-PAB-, wherein:
   a) MC is:

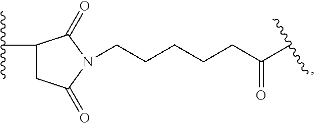 [SMILES: *C(=O)CCCCCN1C(=O)CC(*)C1=O]

b) vc is the dipeptide valine-citrulline, and
c) PAB is:

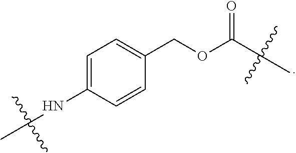 [SMILES: *Nc1ccc(COC(*)=O)cc1]

177. The use of any one of embodiments 174-176, wherein the linker is attached to sulphydryl residues of the anti-TF antibody obtained by partial reduction or full reduction of the anti-TF antibody or antigen-binding fragment thereof.
178. The use of embodiment 177, wherein the linker is attached to MMAE (vcMMAE), wherein the antibody-drug conjugate has the following structure:

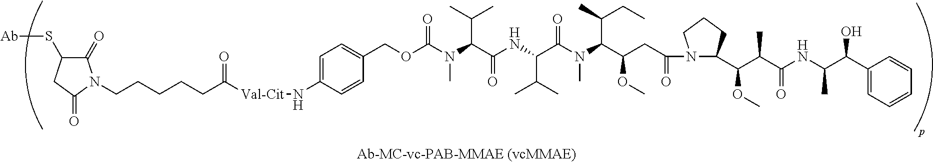

wherein p denotes a number from 1 to 8, S represents a sulphydryl residue of the anti-TF antibody, and Ab designates the anti-TF antibody or antigen-binding fragment thereof.
179. The use of embodiment 178, wherein the average value of p in a population of the antibody-drug conjugates is about 4.
180. The use of any one of embodiments 137-179, wherein the antibody-drug conjugate is tisotumab vedotin.
181. The use of any one of embodiments 137-180, wherein the route of administration for the antibody-drug conjugate is intravenous.
182. The use of any one of embodiments 137-181, wherein the route of administration for the anti-PD-1 antibody or antigen-binding fragment thereof is intravenous.
183. The use of any one of embodiments 137-182, wherein the anti-PD-1 antibody or antigen-binding fragment thereof and the antibody-drug conjugate are administered sequentially.
184. The use of any one of embodiments 137-182, wherein the anti-PD-1 antibody or antigen-binding fragment thereof and the antibody-drug conjugate are administered simultaneously.
185. The use of any one of embodiments 137-184, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express TF.
186. The use of any one of embodiments 137-185, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express PD-L1.
187. The use of any one of embodiments 137-186, wherein a tumor derived from the cancer comprises one or more cells that express PD-L1, PD-L2, or both PD-L1 and PD-L2.
188. The use of any one of embodiments 137-187, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express PD-1.
189. The use of any one of embodiments 137-188, wherein one or more therapeutic effects in the subject is improved after administration of the antibody-drug conjugate and the anti-PD-1 antibody or antigen-binding fragment thereof relative to a baseline.
190. The use of embodiment 189, wherein the one or more therapeutic effects is selected from the group consisting of: size of a tumor derived from the cancer, objective response rate, duration of response, time to response, progression free survival, and overall survival.
191. The use of any one of embodiments 137-190, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody-drug conjugate and the anti-PD-1 antibody or antigen-binding fragment thereof.
192. The use of any one of embodiments 137-191, wherein the objective response rate is at least about 20%, at least about 25%, at least about 30%, at least about 35%, at least about 40%, at least about 45%, at least about 50%, at least about 60%, at least about 70%, or at least about 80%.
193. The use of any one of embodiments 137-192, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
194. The use of any one of embodiments 137-193, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
195. The use of any one of embodiments 137-194, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
196. The use of any one of embodiments 137-195, wherein the subject has one or more adverse events and is further administered an additional therapeutic agent to eliminate or reduce the severity of the one or more adverse events.
197. The use of any one of embodiments 137-196, wherein the subject is at risk of developing one or more adverse events and is further administered an additional therapeutic agent to prevent or reduce the severity of the one or more adverse events.
198. The use of embodiment 196 or embodiment 197, wherein the one or more adverse events is anemia, abdominal pain, hemorrhage, hyperthyroidism, hypothyroidism, hypokalemia, hyponatremia, epistaxis, fatigue, nausea, alopecia, conjunctivitis, keratitis, conjunctival ulceration, constipation, decreased appetite, diarrhea, vomiting, peripheral neuropathy, or general physical health deterioration.
199. The use of any one of embodiments 196-198, wherein the one or more adverse events is a grade 3 or greater adverse event.
200. The use of any one of embodiments 196-198, wherein the one or more adverse events is a serious adverse event.
201. The use of embodiment 196 or embodiment 197, wherein the one or more adverse events is conjunctivitis, conjunctival ulceration, and/or keratitis and the additional agent is a preservative-free lubricating eye drop, an ocular vasoconstrictor, antibiotic, and/or a steroid eye drop.
202. The use of any one of embodiments 137-201, wherein the subject is a human.
203. The use of any one of embodiments 137-202, wherein the antibody-drug conjugate is in a pharmaceutical composition comprising the antibody-drug conjugate and a pharmaceutical acceptable carrier.
204. The use of any one of embodiments 137-203, wherein the anti-PD-1 antibody or antigen-binding fragment thereof is in a pharmaceutical composition comprising the anti-PD-1 antibody or antigen-binding fragment thereof and a pharmaceutical acceptable carrier.
205. A kit comprising:
   (a) an antibody or an antigen-binding fragment thereof, wherein the antibody binds to Programmed Death-1 (PD-1) and inhibits PD-1 activity;
   (b) a dosage ranging from about 0.9 mg/kg to about 2.1 mg/kg of an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof; and
   (c) instructions for use of the anti-PD-1 antibody or antigen-binding fragment thereof and the antibody drug conjugate according to the method of any one of embodiments 1-68 or the antibody-drug conjugate in combination with the anti-PD-1 antibody or the antigen-binding fragment thereof for use of any one of embodiments 69-136 in a method for treating cancer in the subject.
206. An anti-PD-1 antibody or an antigen-binding fragment thereof for use in the treatment of cancer wherein the anti-PD-1 antibody or an antigen-binding fragment thereof is for administration, or to be administered in combination with an antibody-drug conjugate that binds to TF, wherein the antibody-drug conjugate comprises an anti-TF antibody or an antigen-binding fragment thereof conjugated to a monomethyl auristatin or a functional analog thereof or a functional derivative thereof, wherein the anti-PD-1 antibody or the antigen-binding fragment thereof inhibits PD-1 activity, and wherein the anti-PD-1 antibody or antigen-binding fragment thereof comprises the complementary determining regions (CDRs) of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof.
207. The anti-PD-1 antibody or an antigen-binding fragment thereof for use of embodiment 206, wherein the anti-PD-1 antibody or antigen-binding fragment thereof comprises the CDRs of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.
208. The anti-PD-1 antibody or an antigen-binding fragment thereof for use of embodiment 206 or 207, wherein the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof.
209. The anti-PD-1 antibody or an antigen-binding fragment thereof for use of embodiment 206 or 207, wherein the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.
210. The anti-PD-1 antibody or an antigen-binding fragment thereof for use of embodiment 206, wherein the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof.
211. The anti-PD-1 antibody or an antigen-binding fragment thereof for use of embodiment 206, wherein the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.
212. The anti-PD-1 antibody or an antigen-binding fragment thereof for use of any one of embodiments 206-211, wherein the antibody-drug conjugate is administered at a dose ranging from about 0.9 mg/kg to about 2.1 mg/kg.
213. The anti-PD-1 antibody or an antigen-binding fragment thereof for use of embodiment 212, wherein the antibody-drug conjugate is administered at a dose of about 2.0 mg/kg.
214. The anti-PD-1 antibody or an antigen-binding fragment thereof for use of embodiment 212, wherein the antibody-drug conjugate is administered at a dose of 2.0 mg/kg.
215. The anti-PD-1 antibody or an antigen-binding fragment thereof for use of any one of embodiments 206-214, wherein the antibody-drug conjugate is administered once about every 1 week, once about every 2 weeks, once about every 3 weeks or once about every 4 weeks.
216. The anti-PD-1 antibody or an antigen-binding fragment thereof for use of embodiment 215, wherein the antibody-drug conjugate is administered once about every 3 weeks.
217. The anti-PD-1 antibody or an antigen-binding fragment thereof for use of any one of embodiments 206-216, wherein the anti-PD-1 antibody or antigen-binding fragment thereof comprises a heavy chain variable region and a light chain variable region, wherein the heavy chain variable region comprises:
   (i) a CDR-H1 comprising the amino acid sequence of SEQ ID NO:17;
   (ii) a CDR-H2 comprising the amino acid sequence of SEQ ID NO:18; and
   (iii) a CDR-H3 comprising the amino acid sequence of SEQ ID NO:19; and wherein the light chain variable region comprises:
   (i) a CDR-L1 comprising the amino acid sequence of SEQ ID NO:20;
   (ii) a CDR-L2 comprising the amino acid sequence of SEQ ID NO:21; and
   (iii) a CDR-L3 comprising the amino acid sequence of SEQ ID NO:22.
218. The anti-PD-1 antibody or an antigen-binding fragment thereof for use of any one of embodiments 206-217, wherein the anti-PD-1 antibody or antigen-binding fragment thereof comprises a heavy chain variable region comprising an amino acid sequence having at least 85% sequence identity to the amino acid sequence of SEQ ID NO:31 and a light chain variable region comprising an amino acid sequence having at least 85% sequence identity to the amino acid sequence of SEQ ID NO:32.
219. The anti-PD-1 antibody or an antigen-binding fragment thereof for use of any one of embodiments 206-218, wherein the anti-PD-1 antibody or antigen-binding fragment thereof comprises a heavy chain variable region comprising the amino acid sequence of SEQ ID NO:31 and a light chain variable region comprising the amino acid sequence of SEQ ID NO:32.
220. The anti-PD-1 antibody or an antigen-binding fragment thereof for use of any one of embodiments 206-219, wherein the anti-PD-1 antibody or antigen-binding fragment thereof is nivolumab.
221. The anti-PD-1 antibody or an antigen-binding fragment thereof for use of any one of embodiments 206-220, wherein the anti-PD-1 antibody or antigen-binding fragment thereof is administered at a dose ranging from about 0.5 mg/kg to about 4.1 mg/kg.
222. The anti-PD-1 antibody or an antigen-binding fragment thereof for use of any one of embodiments 206-220, wherein the anti-PD-1 antibody or antigen-binding fragment thereof is administered at a flat dose ranging from about 50 mg to about 500 mg.
223. The anti-PD-1 antibody or an antigen-binding fragment thereof for use of any one of embodiments 206-220, wherein the anti-PD-1 antibody or antigen-binding fragment thereof is administered at a flat dose of about 240 mg.
224. The anti-PD-1 antibody or an antigen-binding fragment thereof for use of any one of embodiments 206-220, wherein the anti-PD-1 antibody or antigen-binding fragment thereof is administered at a flat dose of about 480 mg.
225. The anti-PD-1 antibody or an antigen-binding fragment thereof for use of any one of embodiments 206-224, wherein the anti-PD-1 antibody or antigen-binding fragment thereof is administered once about every 1 week, once about every 2 weeks, once about every 3 weeks or once about every 4 weeks.
226. The anti-PD-1 antibody or an antigen-binding fragment thereof for use of embodiment 225, wherein the anti-PD-1 antibody or antigen-binding fragment thereof is administered once about every 2 weeks.
227. The anti-PD-1 antibody or an antigen-binding fragment thereof for use of any one of embodiments 206-226, wherein the cancer is breast cancer.
228. The anti-PD-1 antibody or an antigen-binding fragment thereof for use of any one of embodiments 206-226, wherein the cancer is cervical cancer.
229. The anti-PD-1 antibody or an antigen-binding fragment thereof for use of embodiment 228, wherein the subject is not a candidate for curative therapy.
230. The anti-PD-1 antibody or an antigen-binding fragment thereof for use of embodiment 229, wherein curative therapy comprises radiotherapy and/or exenterative surgery.
231. The anti-PD-1 antibody or an antigen-binding fragment thereof for use of embodiment 228, wherein the subject has not received prior systemic therapy for the cervical cancer.
232. The anti-PD-1 antibody or an antigen-binding fragment thereof for use of any one of embodiment 228-231, wherein the cervical cancer is an adenocarcinoma, an adenosquamous carcinoma or a squamous cell carcinoma.
233. The anti-PD-1 antibody or an antigen-binding fragment thereof for use of any one of embodiments 228-232, wherein the cervical cancer is an advanced stage cervical cancer.
234. The anti-PD-1 antibody or an antigen-binding fragment thereof for use of embodiment 233, wherein the advanced stage cervical cancer is a stage 3 or stage 4 cervical cancer.
235. The anti-PD-1 antibody or an antigen-binding fragment thereof for use of embodiment 233 or 234, wherein the advanced stage cervical cancer is metastatic cervical cancer.
236. The anti-PD-1 antibody or an antigen-binding fragment thereof for use of any one of embodiments 228-235, wherein the cervical cancer is recurrent cervical cancer.
237. The anti-PD-1 antibody or an antigen-binding fragment thereof for use of any one of embodiments 206-236, wherein the monomethyl auristatin is monomethyl auristatin E (MMAE).
238. The anti-PD-1 antibody or an antigen-binding fragment thereof for use of any one of embodiments 206-237, wherein the anti-TF antibody or antigen-binding fragment thereof of the antibody-drug conjugate is a monoclonal antibody or a monoclonal antigen-binding fragment thereof.
239. The anti-PD-1 antibody or an antigen-binding fragment thereof for use of any one of embodiments 206-238, wherein the anti-TF antibody or antigen-binding fragment thereof of the antibody-drug conjugate comprises a heavy chain variable region and a light chain variable region, wherein the heavy chain variable region comprises:
   (i) a CDR-H1 comprising the amino acid sequence of SEQ ID NO:1;
   (ii) a CDR-H2 comprising the amino acid sequence of SEQ ID NO:2; and
   (iii) a CDR-H3 comprising the amino acid sequence of SEQ ID NO:3; and wherein the light chain variable region comprises:
   (i) a CDR-L1 comprising the amino acid sequence of SEQ ID NO:4;
   (ii) a CDR-L2 comprising the amino acid sequence of SEQ ID NO:5; and
   (iii) a CDR-L3 comprising the amino acid sequence of SEQ ID NO:6.
240. The anti-PD-1 antibody or an antigen-binding fragment thereof for use of any one of embodiments 206-239, wherein the anti-TF antibody or antigen-binding fragment thereof of the antibody-drug conjugate comprises a heavy chain variable region comprising an amino acid sequence having at least 85% sequence identity to the amino acid sequence of SEQ ID NO:7 and a light chain variable region comprising an amino acid sequence having at least 85% sequence identity to the amino acid sequence of SEQ ID NO:8.
241. The anti-PD-1 antibody or an antigen-binding fragment thereof for use of any one of embodiments 206-240, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.
242. The anti-PD-1 antibody or an antigen-binding fragment thereof for use of any one of embodiments 206-241, wherein the anti-TF antibody of the antibody-drug conjugate is tisotumab.
243. The anti-PD-1 antibody or an antigen-binding fragment thereof for use of any one of embodiments 206-242, wherein the antibody-drug conjugate further comprises a linker between the anti-TF antibody or antigen-binding fragment thereof and the monomethyl auristatin.
244. The anti-PD-1 antibody or an antigen-binding fragment thereof for use of embodiment 243, wherein the linker is a cleavable peptide linker.
245. The anti-PD-1 antibody or an antigen-binding fragment thereof for use of embodiment 244, wherein the cleavable peptide linker has a formula: -MC-vc-PAB-, wherein:
   a) MC is:

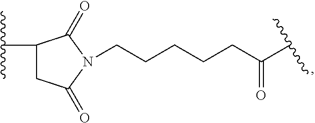 [SMILES: *C(=O)CCCCCN1C(=O)CC(*)C1=O]

b) vc is the dipeptide valine-citrulline, and
c) PAB is:

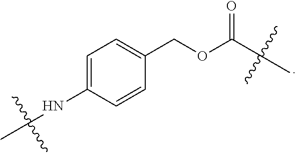 [SMILES: *Nc1ccc(COC(*)=O)cc1]

246. The anti-PD-1 antibody or an antigen-binding fragment thereof for use of any one of embodiments 243-245, wherein the linker is attached to sulphydryl residues of the anti-TF antibody obtained by partial reduction or full reduction of the anti-TF antibody or antigen-binding fragment thereof.
247. The anti-PD-1 antibody or an antigen-binding fragment thereof for use of embodiment 246, wherein the linker is attached to MMAE (vcMMAE), wherein the antibody-drug conjugate has the following structure:

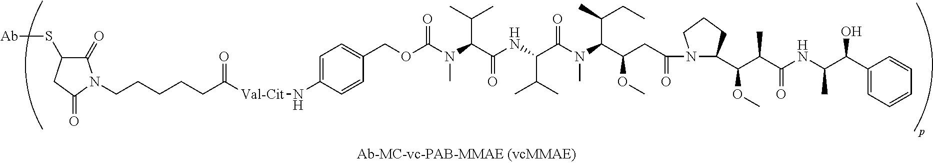

wherein p denotes a number from 1 to 8, S represents a sulphydryl residue of the anti-TF antibody, and Ab designates the anti-TF antibody or antigen-binding fragment thereof.
248. The anti-PD-1 antibody or an antigen-binding fragment thereof for use of embodiment 247, wherein the average value of p in a population of the antibody-drug conjugates is about 4.
249. The anti-PD-1 antibody or an antigen-binding fragment thereof for use of any one of embodiments 206-248, wherein the antibody-drug conjugate is tisotumab vedotin.
250. The anti-PD-1 antibody or an antigen-binding fragment thereof for use of any one of embodiments 206-249, wherein the route of administration for the antibody-drug conjugate is intravenous.
251. The anti-PD-1 antibody or an antigen-binding fragment thereof for use of any one of embodiments 206-250, wherein the route of administration for the anti-PD-1 antibody or antigen-binding fragment thereof is intravenous.
252. The anti-PD-1 antibody or an antigen-binding fragment thereof for use of any one of embodiments 206-251, wherein the anti-PD-1 antibody or antigen-binding fragment thereof and the antibody-drug conjugate are administered sequentially.
253. The anti-PD-1 antibody or an antigen-binding fragment thereof for use of any one of embodiments 206-251, wherein the anti-PD-1 antibody or antigen-binding fragment thereof and the antibody-drug conjugate are administered simultaneously.
254. The anti-PD-1 antibody or an antigen-binding fragment thereof for use of any one of embodiments 206-253, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express TF.
255. The anti-PD-1 antibody or an antigen-binding fragment thereof for use of any one of embodiments 206-254, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express PD-L1.
256. The anti-PD-1 antibody or an antigen-binding fragment thereof for use of any one of embodiments 206-255, wherein a tumor derived from the cancer comprises one or more cells that express PD-L1, PD-L2, or both PD-L1 and PD-L2.
257. The anti-PD-1 antibody or an antigen-binding fragment thereof for use of any one of embodiments 206-256, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express PD-1.
258. The anti-PD-1 antibody or an antigen-binding fragment thereof for use of any one of embodiments 206-257, wherein one or more therapeutic effects in the subject is improved after administration of the antibody-drug conjugate and the anti-PD-1 antibody or antigen-binding fragment thereof relative to a baseline.
259. The anti-PD-1 antibody or an antigen-binding fragment thereof for use of embodiment 258, wherein the one or more therapeutic effects is selected from the group consisting of: size of a tumor derived from the cancer, objective response rate, duration of response, time to response, progression free survival, and overall survival.
260. The anti-PD-1 antibody or an antigen-binding fragment thereof for use of any one of embodiments 206-259, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody-drug conjugate and the anti-PD-1 antibody or antigen-binding fragment thereof.
261. The anti-PD-1 antibody or an antigen-binding fragment thereof for use of any one of embodiments 206-260, wherein the objective response rate is at least about 20%, at least about 25%, at least about 30%, at least about 35%, at least about 40%, at least about 45%, at least about 50%, at least about 60%, at least about 70%, or at least about 80%.
262. The anti-PD-1 antibody or an antigen-binding fragment thereof for use of any one of embodiments 206-261, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
263. The anti-PD-1 antibody or an antigen-binding fragment thereof for use of any one of embodiments 206-262, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
264. The anti-PD-1 antibody or an antigen-binding fragment thereof for use of any one of embodiments 206-263, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
265. The anti-PD-1 antibody or an antigen-binding fragment thereof for use of any one of embodiments 206-264, wherein the subject has one or more adverse events and is further administered an additional therapeutic agent to eliminate or reduce the severity of the one or more adverse events.
266. The anti-PD-1 antibody or an antigen-binding fragment thereof for use of any one of embodiments 206-265, wherein the subject is at risk of developing one or more adverse events and is further administered an additional therapeutic agent to prevent or reduce the severity of the one or more adverse events.
267. The anti-PD-1 antibody or an antigen-binding fragment thereof for use of embodiment 265 or embodiment 266, wherein the one or more adverse events is anemia, abdominal pain, hemorrhage, hyperthyroidism, hypothyroidism, hypokalemia, hyponatremia, epistaxis, fatigue, nausea, alopecia, conjunctivitis, keratitis, conjunctival ulceration, constipation, decreased appetite, diarrhea, vomiting, peripheral neuropathy, or general physical health deterioration.
268. The anti-PD-1 antibody or an antigen-binding fragment thereof for use of any one of embodiments 265-267, wherein the one or more adverse events is a grade 3 or greater adverse event.
269. The anti-PD-1 antibody or an antigen-binding fragment thereof for use of any one of embodiments 265-267, wherein the one or more adverse events is a serious adverse event.
270. The anti-PD-1 antibody or an antigen-binding fragment thereof for use of embodiment 265 or embodiment 266, wherein the one or more adverse events is conjunctivitis, conjunctival ulceration, and/or keratitis and the additional agent is a preservative-free lubricating eye drop, an ocular vasoconstrictor, antibiotic, and/or a steroid eye drop.
271. The anti-PD-1 antibody or an antigen-binding fragment thereof for use of any one of embodiments 206-270, wherein the subject is a human.
272. The anti-PD-1 antibody or an antigen-binding fragment thereof for use of any one of embodiments 206-271, wherein the antibody-drug conjugate is in a pharmaceutical composition comprising the antibody-drug conjugate and a pharmaceutical acceptable carrier.
273. The anti-PD-1 antibody or an antigen-binding fragment thereof for use of any one of embodiments 206-272, wherein the anti-PD-1 antibody or antigen-binding fragment thereof is in a pharmaceutical composition comprising the anti-PD-1 antibody or antigen-binding fragment thereof and a pharmaceutical acceptable carrier.
274. Use of an anti-PD-1 antibody or an antigen-binding fragment thereof for the manufacture of a medicament for treating cancer in a subject, wherein the medicament is for use in combination with an antibody-drug conjugate that binds to TF, wherein the antibody-drug conjugate comprises an anti-TF antibody or an antigen-binding fragment thereof conjugated to a monomethyl auristatin or a functional analog thereof or a functional derivative thereof, wherein the anti-PD-1 antibody or the antigen-binding fragment thereof inhibits PD-1 activity, and wherein the anti-PD-1 antibody or antigen-binding fragment thereof comprises the complementary determining regions (CDRs) of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof.
275. The use of embodiment 274, wherein the anti-PD-1 antibody or antigen-binding fragment thereof comprises the CDRs of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.
276. The use of embodiment 274 or 275, wherein the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof.
277. The use of embodiment 274 or 275, wherein the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.
278. The use of embodiment 274, wherein the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof.
279. The use of embodiment 274, wherein the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.
280. The use of any one of embodiments 274-279, wherein the antibody-drug conjugate is administered at a dose ranging from about 0.9 mg/kg to about 2.1 mg/kg.
281. The use of embodiment 280, wherein the antibody-drug conjugate is administered at a dose of about 2.0 mg/kg.
282. The use of embodiment 280, wherein the antibody-drug conjugate is administered at a dose of 2.0 mg/kg.
283. The use of any one of embodiments 274-282, wherein the antibody-drug conjugate is administered once about every 1 week, once about every 2 weeks, once about every 3 weeks or once about every 4 weeks.
284. The use of embodiment 283, wherein the antibody-drug conjugate is administered once about every 3 weeks.
285. The use of any one of embodiments 274-284, wherein the anti-PD-1 antibody or antigen-binding fragment thereof comprises a heavy chain variable region and a light chain variable region, wherein the heavy chain variable region comprises:
   (i) a CDR-H1 comprising the amino acid sequence of SEQ ID NO:17;
   (ii) a CDR-H2 comprising the amino acid sequence of SEQ ID NO:18; and
   (iii) a CDR-H3 comprising the amino acid sequence of SEQ ID NO:19; and wherein the light chain variable region comprises:
   (i) a CDR-L1 comprising the amino acid sequence of SEQ ID NO:20;
   (ii) a CDR-L2 comprising the amino acid sequence of SEQ ID NO:21; and
   (iii) a CDR-L3 comprising the amino acid sequence of SEQ ID NO:22.
286. The use of any one of embodiments 274-285, wherein the anti-PD-1 antibody or antigen-binding fragment thereof comprises a heavy chain variable region comprising an amino acid sequence having at least 85% sequence identity to the amino acid sequence of SEQ ID NO:31 and a light chain variable region comprising an amino acid sequence having at least 85% sequence identity to the amino acid sequence of SEQ ID NO:32.
287. The use of any one of embodiments 274-286, wherein the anti-PD-1 antibody or antigen-binding fragment thereof comprises a heavy chain variable region comprising the amino acid sequence of SEQ ID NO:31 and a light chain variable region comprising the amino acid sequence of SEQ ID NO:32.
288. The use of any one of embodiments 274-287, wherein the anti-PD-1 antibody or antigen-binding fragment thereof is nivolumab.
289. The use of any one of embodiments 274-288, wherein the anti-PD-1 antibody or antigen-binding fragment thereof is administered at a dose ranging from about 0.5 mg/kg to about 4.1 mg/kg.
290. The use of any one of embodiments 274-288, wherein the anti-PD-1 antibody or antigen-binding fragment thereof is administered at a flat dose ranging from about 50 mg to about 500 mg.
291. The use of any one of embodiments 274-288, wherein the anti-PD-1 antibody or antigen-binding fragment thereof is administered at a flat dose of about 240 mg.
292. The use of any one of embodiments 274-288, wherein the anti-PD-1 antibody or antigen-binding fragment thereof is administered at a flat dose of about 480 mg.
293. The use of any one of embodiments 274-292, wherein the anti-PD-1 antibody or antigen-binding fragment thereof is administered once about every 1 week, once about every 2 weeks, once about every 3 weeks or once about every 4 weeks.
294. The use of embodiment 293, wherein the anti-PD-1 antibody or antigen-binding fragment thereof is administered once about every 2 weeks.
295. The use of any one of embodiments 274-294, wherein the cancer is breast cancer.
296. The use of any one of embodiments 274-294, wherein the cancer is cervical cancer.
297. The use of embodiment 296, wherein the subject is not a candidate for curative therapy.
298. The use of embodiment 297, wherein curative therapy comprises radiotherapy and/or exenterative surgery.
299. The use of embodiment 296, wherein the subject has not received prior systemic therapy for the cervical cancer.
300. The use of any one of embodiment 296-299, wherein the cervical cancer is an adenocarcinoma, an adenosquamous carcinoma or a squamous cell carcinoma.
301. The use of any one of embodiments 296-300, wherein the cervical cancer is an advanced stage cervical cancer.
302. The use of embodiment 301, wherein the advanced stage cervical cancer is a stage 3 or stage 4 cervical cancer.
303. The use of embodiment 301 or 302, wherein the advanced stage cervical cancer is metastatic cervical cancer.
304. The use of any one of embodiments 296-303, wherein the cervical cancer is recurrent cervical cancer.
305. The use of any one of embodiments 274-304, wherein the monomethyl auristatin is monomethyl auristatin E (MMAE).
306. The use of any one of embodiments 274-305, wherein the anti-TF antibody or antigen-binding fragment thereof of the antibody-drug conjugate is a monoclonal antibody or a monoclonal antigen-binding fragment thereof.
307. The use of any one of embodiments 274-306, wherein the anti-TF antibody or antigen-binding fragment thereof of the antibody-drug conjugate comprises a heavy chain variable region and a light chain variable region, wherein the heavy chain variable region comprises:
   (i) a CDR-H1 comprising the amino acid sequence of SEQ ID NO:1;
   (ii) a CDR-H2 comprising the amino acid sequence of SEQ ID NO:2; and
   (iii) a CDR-H3 comprising the amino acid sequence of SEQ ID NO:3; and wherein the light chain variable region comprises:
   (i) a CDR-L1 comprising the amino acid sequence of SEQ ID NO:4;
   (ii) a CDR-L2 comprising the amino acid sequence of SEQ ID NO:5; and
   (iii) a CDR-L3 comprising the amino acid sequence of SEQ ID NO:6.
308. The use of any one of embodiments 274-307, wherein the anti-TF antibody or antigen-binding fragment thereof of the antibody-drug conjugate comprises a heavy chain variable region comprising an amino acid sequence having at least 85% sequence identity to the amino acid sequence of SEQ ID NO:7 and a light chain variable region comprising an amino acid sequence having at least 85% sequence identity to the amino acid sequence of SEQ ID NO:8.
309. The use of any one of embodiments 274-308, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.
310. The use of any one of embodiments 274-309, wherein the anti-TF antibody of the antibody-drug conjugate is tisotumab.
311. The use of any one of embodiments 274-310, wherein the antibody-drug conjugate further comprises a linker between the anti-TF antibody or antigen-binding fragment thereof and the monomethyl auristatin.
312. The use of embodiment 311, wherein the linker is a cleavable peptide linker.
313. The use of embodiment 312, wherein the cleavable peptide linker has a formula: -MC-vc-PAB-, wherein:
   a) MC is:

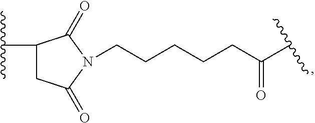 [SMILES: *C(=O)CCCCCN1C(=O)CC(*)C1=O]

b) vc is the dipeptide valine-citrulline, and
c) PAB is:

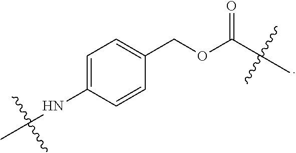 [SMILES: *Nc1ccc(COC(*)=O)cc1]

314. The use of any one of embodiments 311-313, wherein the linker is attached to sulphydryl residues of the anti-TF antibody obtained by partial reduction or full reduction of the anti-TF antibody or antigen-binding fragment thereof.
315. The use of embodiment 314, wherein the linker is attached to MMAE (vcMMAE), wherein the antibody-drug conjugate has the following structure:

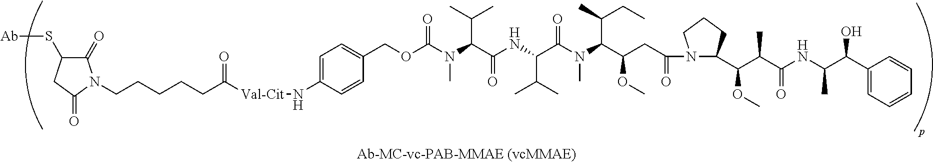

wherein p denotes a number from 1 to 8, S represents a sulphydryl residue of the anti-TF antibody, and Ab designates the anti-TF antibody or antigen-binding fragment thereof.
316. The use of embodiment 315, wherein the average value of p in a population of the antibody-drug conjugates is about 4.
317. The use of any one of embodiments 274-316, wherein the antibody-drug conjugate is tisotumab vedotin.
318. The use of any one of embodiments 274-317, wherein the route of administration for the antibody-drug conjugate is intravenous.
319. The use of any one of embodiments 274-318, wherein the route of administration for the anti-PD-1 antibody or antigen-binding fragment thereof is intravenous.
320. The use of any one of embodiments 274-319, wherein the anti-PD-1 antibody or antigen-binding fragment thereof and the antibody-drug conjugate are administered sequentially.
321. The use of any one of embodiments 274-319, wherein the anti-PD-1 antibody or antigen-binding fragment thereof and the antibody-drug conjugate are administered simultaneously.
322. The use of any one of embodiments 274-321, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express TF.
323. The use of any one of embodiments 274-322, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express PD-L1.
324. The use of any one of embodiments 274-323, wherein a tumor derived from the cancer comprises one or more cells that express PD-L1, PD-L2, or both PD-L1 and PD-L2.
325. The use of any one of embodiments 274-324, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express PD-1.
326. The use of any one of embodiments 274-325, wherein one or more therapeutic effects in the subject is improved after administration of the antibody-drug conjugate and the anti-PD-1 antibody or antigen-binding fragment thereof relative to a baseline.
327. The use of embodiment 326, wherein the one or more therapeutic effects is selected from the group consisting of: size of a tumor derived from the cancer, objective response rate, duration of response, time to response, progression free survival, and overall survival.
328. The use of any one of embodiments 274-327, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody-drug conjugate and the anti-PD-1 antibody or antigen-binding fragment thereof.
329. The use of any one of embodiments 274-328, wherein the objective response rate is at least about 20%, at least about 25%, at least about 30%, at least about 35%, at least about 40%, at least about 45%, at least about 50%, at least about 60%, at least about 70%, or at least about 80%.
330. The use of any one of embodiments 274-329, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
331. The use of any one of embodiments 274-330, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
332. The use of any one of embodiments 274-331, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate and the anti-PD-1 antibody or antigen-binding fragment thereof.
333. The use of any one of embodiments 274-332, wherein the subject has one or more adverse events and is further administered an additional therapeutic agent to eliminate or reduce the severity of the one or more adverse events.
334. The use of any one of embodiments 274-333, wherein the subject is at risk of developing one or more adverse events and is further administered an additional therapeutic agent to prevent or reduce the severity of the one or more adverse events.
335. The use of embodiment 333 or embodiment 334, wherein the one or more adverse events is anemia, abdominal pain, hemorrhage, hyperthyroidism, hypothyroidism, hypokalemia, hyponatremia, epistaxis, fatigue, nausea, alopecia, conjunctivitis, keratitis, conjunctival ulceration, constipation, decreased appetite, diarrhea, vomiting, peripheral neuropathy, or general physical health deterioration.
336. The use of any one of embodiments 333-335, wherein the one or more adverse events is a grade 3 or greater adverse event.
337. The use of any one of embodiments 333-335, wherein the one or more adverse events is a serious adverse event.
338. The use of embodiment 333 or embodiment 334, wherein the one or more adverse events is conjunctivitis, conjunctival ulceration, and/or keratitis and the additional agent is a preservative-free lubricating eye drop, an ocular vasoconstrictor, antibiotic, and/or a steroid eye drop.
339. The use of any one of embodiments 274-338, wherein the subject is a human.
340. The use of any one of embodiments 274-339, wherein the antibody-drug conjugate is in a pharmaceutical composition comprising the antibody-drug conjugate and a pharmaceutical acceptable carrier.
341. The use of any one of embodiments 274-340, wherein the anti-PD-1 antibody or antigen-binding fragment thereof is in a pharmaceutical composition comprising the anti-PD-1 antibody or antigen-binding fragment thereof and a pharmaceutical acceptable carrier.

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1: MMAE Elicits Hallmark Characteristics Associated with Immunogenic Cell Death in a Cervical Cancer Cell Line

Immunogenic cell death (ICD) is a regulated cell death program that is highlighted by the production and exposure of pro-inflammatory signals that leads to the generation of immune responses against the apoptotic tumor cells. ICD is characterized by: 1) exposure of endoplasmic reticulum (ER)-resident chaperone proteins on the surface of tumor cells; 2) secretion of ATP; and 3) secretion of HMGB1. Induction of ER stress is critical for regulating these 3 processes and has been shown to be elicited by antibody-drug conjugates (ADCs) wherein the conjugated drug is MMAE.

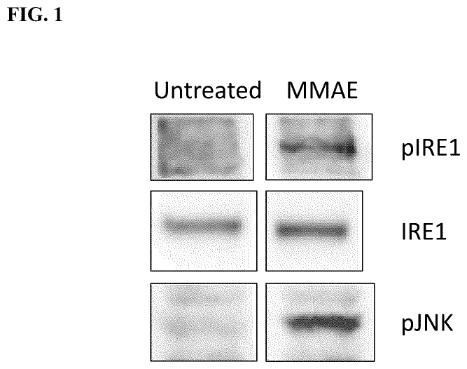

HeLa cells, a cervical cancer cell line, were cultured in Minimum Essential Medium (MEM) with 10% FBS, 10 mM HEPES, 1 mM sodium pyruvate, 2 mM L-glutamine, penicillin (100 U/ml), and streptomycin (100 μg/ml). HeLa cells were treated with 100 nM MMAE for 16 hours and harvested in radioimmunoprecipitation assay buffer (RIPA) buffer for western blot analysis. Treatment with MMAE led to phosphorylation of the serine threonine kinase IRE1, indicating activation of ER stress. Severe ER stress is a prerequisite to the exposure of pro-phagocytic signals on the surface of tumor cells, and can be indicated by activation of JNK signaling by phosphorylated IRE1. As demonstrated herein, treatment with MMAE elicited severe ER stress by phosphorylation of IRE1 and JNK (FIG. 1).

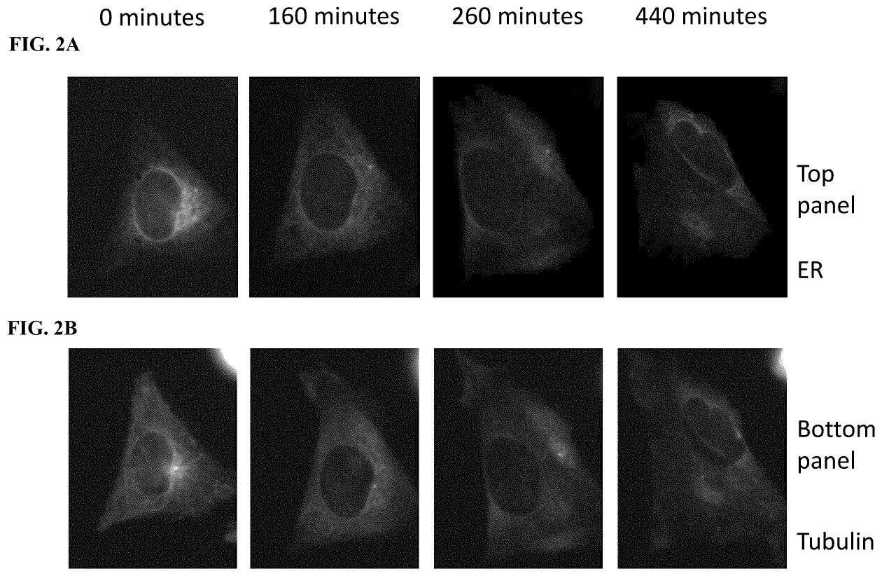

Treatment of HeLa cells with MMAE led to disassembly of the microtubule network and subsequent ER mislocalization. HeLa cells were transduced with a baculovirus encoding RFP-labeled Tubulin (CellLight Tubulin-RFP, ThermoFisher Scientific) and an ER-binding dye (ER-ID Green, Enzo Life Sciences). Cells were treated with 100 nM MMAE and imaged over time in the present of MMAE. Within 2 hours, fragmentation and disassembly of the microtubule network became evident, concurrent with the breakdown of the perinuclear organized ER lattice (FIGS. 2A and B). The condensed and mislocalized ER skeleton indicated severe ER stress within 8 hours.

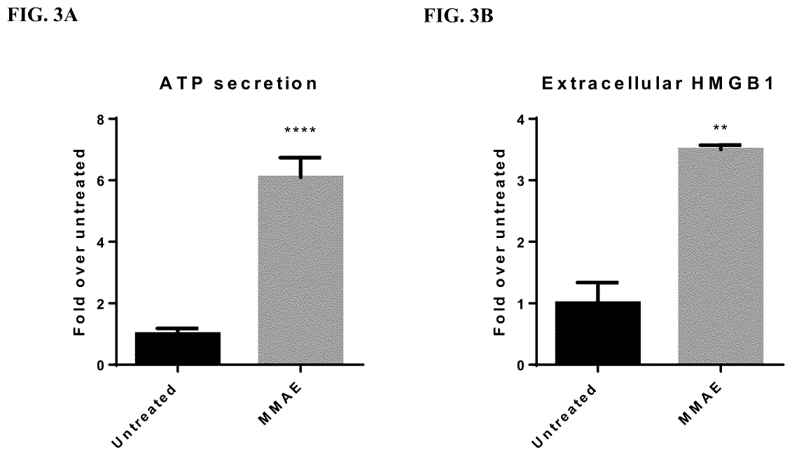

Induction of ICD is also characterized by the secretion of ATP and HMGB1. Extracellular ATP serves as a strong chemotactic signal, promoting immune cell migration to the tumor site. Upon arrival, extracellular HMGB1 signals through various pro-inflammatory receptors (TLR2, TLR4, RAGE) to activate antigen-presenting cells, thereby promoting immune activity within the tumor. As demonstrated herein, treatment of HeLa cells with 100 nM MMAE led to increased secretion of ATP and HMGB1 over a period of 24 hours (FIGS. 3A and B; **p<0.01, ****p<0.0001).

While the sequence of events of ADC binding to antigen positive cells, cleavage and release of the MMAE payload, and subsequent cell death is the primary mechanism of tisotumab vedotin functionality, each step in this process can evoke additional and distinct modalities that may contribute to overall antitumor activity. The MMAE cytotoxic payload connected to tisotumab vedotin disrupts microtubules which results in subsequent endoplasmic reticulum (ER) stress that drives exposure of immune activating molecules that can promote a T-cell response. The effect of MMAE on a cervical cancer cell line as shown in this example, demonstrates induction of the ER stress pathway and exposure of immune activating molecules. Accordingly, the T-cell response that may occur following tumor cell death with tisotumab vedotin could amplify the effect of treatment with a checkpoint inhibitor.

### Example 2: Anti-Tumor Activity of Tisotumab Vedotin in Combination with an Anti-PD-1 Monoclonal Antibody in a Xenograft Model in Humanized Mice

Tisotumab vedotin is an antibody-drug conjugate comprising an antibody that binds to tissue factor (TF), a protease-cleavable linker, and the microtubule disrupting agent MMAE. TF is a protein aberrantly expressed in a wide number of tumors including cervical cancer and is associated with poor prognosis. See Forster Y et al. Clin Chim Acta. 2006; 364 (1-2):12-21 and Cocco E et al. BMC Cancer. 2011; 11:263. Tisotumab vedotin selectively targets TF to deliver a clinically validated toxic payload to tumor cells. See Breij E C et al. Cancer Res. 2014; 74(4):1214-1226 and Chu A J. Int J Inflam. 2011; 2011. doi: 10.4061/2011/367284.

The anti-PD-1 antibody, nivolumab (OPDIVO®), is a checkpoint inhibitor that is a standard of care therapy alone or in combination with chemotherapies in multiple tumor indications. The combination of tisotumab vedotin with an anti-PD-1 antibody such as nivolumab is evaluated herein for the treatment of cancer.

### Materials and Methods

The in vivo anti-tumor efficacy of tisotumab vedotin in combination with an anti-PD-1 monoclonal antibody is evaluated in NOD.Cg-Prkdc^{scid} Il2rg^{tm1Wj1}/SzJ (NSG) immunodeficient mice (The Jackson Laboratory, Stock No. 005557), humanized by engraftment with human CD34⁺ hematopoietic stem cells (Jackson Laboratories, Sacramento). Mice are subcutaneously inoculated with 5×10⁶ MDA-MB-231 cells (breast adenocarcinoma; American Tissue Culture Collection (ATCC), cat. no. HTB-26), in 100 μL phosphate-buffered saline (PBS). Before inoculation, cells are cultured in DMEM with high glucose and HEPES without L-glutamine (Lonza, cat. no. BE12-709F), 10% (v/v) donor bovine serum with iron New Zealand Origin (Thermo Fisher Scientific, DBSI, cat. no. 10371-029), 2 mM L-glutamine (Lonza, cat. no. BE17-605E), 1 mM Na-pyruvate (Lonza, cat. no. BE13-115E), MEM non-essential amino acids (Life Technologies, cat. no. 11140) and 1% (v/v) penicillin/streptomycin (Lonza, cat. no. DE17-603E), in CellSTACK culture chambers (Corning, cat. no. 3313).

Tumor size is determined by caliper measurement at least two times a week and tumor volume is calculated as 0.52×length×width². When tumors reach the size of 100 mm³ mice are randomized in 7 groups (8 mice per treatment group) based on mouse cohort and tumor size (Table 1). Mice are treated with tisotumab vedotin alone, intravenously, or in combination with an anti-PD-1 antibody (i.e., nivolumab, OPDIVO®) or with anti-PD-1 antibody alone. Mice in control groups are administered 1 mg/kg of IgG1 isotype control antibody or IgG1 isotype control antibody conjugated to MMAE intravenously, weekly for a maximum of five treatments (Table 1). The IgG1 isotype control antibody is the b12 antibody which is known to bind to HIV-1 gp120. Mice are observed for clinical signs of illness at least twice a week. Mice are housed in individually ventilated (IVC) cages, five mice per cage and identified by ear tags.

TABLE 1
Trial design

| | | Route of | |
|---|---|---|---|
| Group | Treatment | administration | Number of mice |
| 1 | IgG1 control | IV | 8 |
| 2 | IgGl-MMAE | IV | 8 |
| | control | | |
| 3 | ADC | IV | 8 |
| 4 | PD-1 | IV | 8 |
| 5 | ADC + | IV | 8 |
| | PD-1 | IV | |
| 6 | ADC | IV | 8 |
| 7 | ADC + | IV | 8 |
| | PD-1 | IV | |

IgG1 control indicates the IgG1 b12 antibody that binds to HIV-1 gp120 and is used as an IgG1 isotype control; IgG1-MMAE control indicates the IgG1 b12 antibody conjugated to MMAE; ADC indicates anti-TF antibody conjugated to MMAE; and PD-1 indicates anti-PD-1 antibody; IV indicates intravenous administration; and IP indicates intraperitoneal administration.

To determine whether there are statistically significant differences between tumor burden in control and treatment groups, tumor burden in the treatment groups is compared with those in the control groups (e.g., control antibody (e.g., IgG1 control or anti-PD-1 antibody) or control antibody-drug conjugate (e.g., tisotumab vedotin or IgG1-MMAE)). Statistical comparison of tumor burden is performed using Mann-Whitney analysis on the last day that all treatment groups are intact. Kaplan-Meier analysis is performed based on tumor volume (>500 mm³).

### Example 3: Anti-Tumor Activity of Tisotumab Vedotin in Combination with an Anti-PD-1 Monoclonal Antibody in a Patient-Derived Xenograft Model in Humanized Mice

Nivolumab has been tested in patients with cervical cancer. Nivolumab 240 mg Q2W was administered to 19 patients with previously treated, advanced cervical cancer. The objective response rate was 26%. See Hollebecque A, et al. Abstract 5504. Presented at: ASCO Annual Meeting; Jun. 2-6, 2017; Chicago. The combination of tisotumab vedotin with an anti-PD-1 antibody such as nivolumab is evaluated herein for the treatment of cervical cancer.

### Materials and Methods

The in vivo anti-tumor efficacy of tisotumab vedotin in combination with an anti-PD-1 monoclonal antibody is evaluated in an animal model such as in NOD.Cg-Prkdc^{scid} Il2rg^{tm1Wj1} (NSG) immunodeficient mice or NOD-Prkdc^{em26Cd52}Il2rg^{em26Cd22} (NCG) immunodeficient mice humanized by engraftment with human CD34⁺ hematopoietic stem cells. Patient-derived xenografts (PDX) are derived from tumor specimens from cancer patients. Establishment and characterization of the PDX model is performed following the primary implantation into nude mice. Tumor xenografts are passaged approximately three to five times until establishment of stable growth patterns. Tumor fragments are obtained from xenografts in serial passage in nude mice. Tumors are cut into fragments of 4-5 mm diameter and placed in phosphate-buffered saline (PBS) until subcutaneous implantation. Cervical cancer PDX models (HUPRIME® cervical xenograft model CV1802 and CV2302; Crown Bioscience Inc.) are used in this experiment. Tumor size is determined by caliper measurement at least two times a week and tumor volume is calculated as 0.52×length×width². When tumors reach the volume of 150-250 mm³, mice are randomized in 7 groups per model (10 mice per treatment group), based on tumor volume. Mice are treated with intravenous injections of tisotumab vedotin alone (e.g., at two dose levels between 0.5 mg/kg and 4 mg/kg, weekly) or in combination with an anti-PD-1 monoclonal antibody (e.g., nivolumab, OPDIVO®) or with anti-PD-1 antibody alone (e.g., nivolumab, OPDIVO®). In one example, when the HUPRIME® cervical xenograft model CV2320 is used, mice are treated with intravenous injections of tisotumab vedotin alone at a dose of 4 mg/kg or 2 mg/kg, or in combination with an anti-PD-1 monoclonal antibody (e.g., nivolumab) until a maximum amount of treatment is reached (e.g., five treatments). HUPRIME® cervical xenograft model CV2320 treated with anti-PD-1 monoclonal antibody alone (e.g., nivolumab) are provided until a maximum amount of treatment is reached (e.g., five treatments). In another example, when the HUPRIME® cervical xenograft model CV1802 is used, mice are treated with intravenous injections of tisotumab vedotin alone at a dose of 1 mg/kg or 0.5 mg/kg, or in combination with an anti-PD-1 monoclonal antibody (e.g., nivolumab) until a maximum amount of treatment is reached (e.g., five treatments). HUPRIME® cervical xenograft model CV1802 treated with anti-PD-1 monoclonal antibody alone (e.g., nivolumab) are provided until a maximum amount of treatment is reached (e.g., five treatments). Mice are observed for clinical signs of illness at least twice a week. Mice are housed in individually ventilated (IVC) cages, five mice per cage and identified by ear tags.

To determine whether there are statistically significant differences between tumor volumes in control and treatment groups, tumor volumes in the treatment groups are compared with those in the control groups (e.g., control antibody (e.g., IgG1 control or anti-PD-1 antibody) or control antibody-drug conjugate (e.g., tisotumab vedotin or IgG1-MMAE)), using Mann-Whitney analysis at the last day that all groups are intact. Tumor volumes in mice treated with both tisotumab vedotin and anti-PD-1 antibody are compared with those in mice treated with either control antibody alone (e.g., IgG1 control or anti-PD-1 antibody) or control antibody-drug conjugate alone (e.g., tisotumab vedotin or IgG1-MMAE) and analyzed such as by using Mantel-Cox analysis on Kaplan-Meier plots.

### Example 4: Anti-Tumor Activity of Tisotumab Vedotin in Combination with an Anti-PD-1 Monoclonal Antibody in a Syngeneic Tumor Model

Mouse tumor cells are transfected with plasmid constructs encoding human tissue factor (TF) and sgRNA-guided Cas9 nuclease (sgRNA/Cas9) to generate a murine cell line that expresses the human TF. Fluorescence activated cell sorting (FACS) yields a clonal population of murine tumor cells that stably express human TF, these cells are then treated with lpg to 5 μg per ml of tisotumab vedotin or 100 nM of MMAE for 4 days. In order to prepare dying cells for immunization, treated murine tumor cells are overlaid atop Histopaque, and centrifuged at 2000 g for 30 minutes. Dead and dying cells are pelleted underneath the Histopaque layer, and viability assessed by trypan blue exclusion. A sample with approximately <20% live cells as measured by trypan blue exclusion is obtained. Flash-frozen tumor cells are prepared by submerging the cells in liquid nitrogen for 10 seconds, followed by immersion in 37° C. water until completely thawed. The liquid nitrogen freeze-thaw process is repeated 5 times. Dead and dying human TF positive tumor cells are resuspended in phosphate buffered saline (PBS) and 2×10⁶ cells are injected into the peritoneum of immune-competent Balb/c mice. Seven days later, mice receive a second immunization with dead and dying cells prepared in the same manner.

Fourteen days after initial immunization with the dead and dying human TF positive tumor cells, mice are subcutaneously implanted with 5×10⁶ wild-type tumor cells and monitored for tumor growth. Mice that are immunized with tisotumab vedotin-killed tumor cells or MMAE-killed tumor cells experience delayed tumor growth and increased survival. As these effects occur in the absence of any administered therapeutic agent, the administration of cells killed by tisotumab vedotin or MMAE is sufficient to generate long-lasting protective immune memory against subsequent tumor cell challenge. Protective immune memory is amplified by treating these mice with tisotumab vedotin in combination with an antibody that binds to murine PD-1. This combination treatment increases the number of mice that are cured by subsequent tumor challenge.

### Example 5: Cells from Multiple Tissues Exposed to Tisotumab Vedotin ADC and MMAE Undergo Cell Death and Release ATP and HMGB1

Immunogenic cell death (ICD) is a mode of apoptosis that generates immune responses against the apoptotic cancerous cells. Proteins normally found within the endoplasmic reticulum (ER) become exposed on the cell surface, leading to increased phagocytic uptake and presentation of tumor antigens to T cells in order to prime the adaptive immune system. As such, ICD induction enables the immune system to recognize and mount cytotoxic activity against tumors.

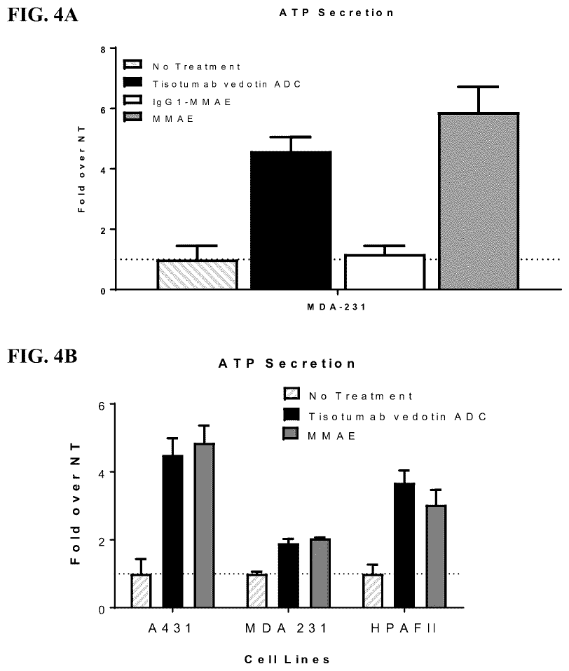

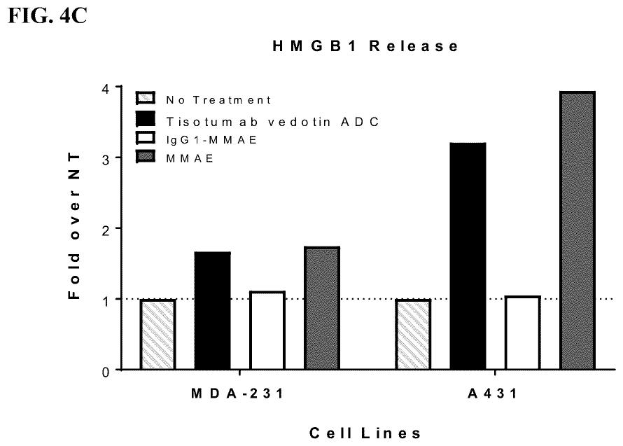

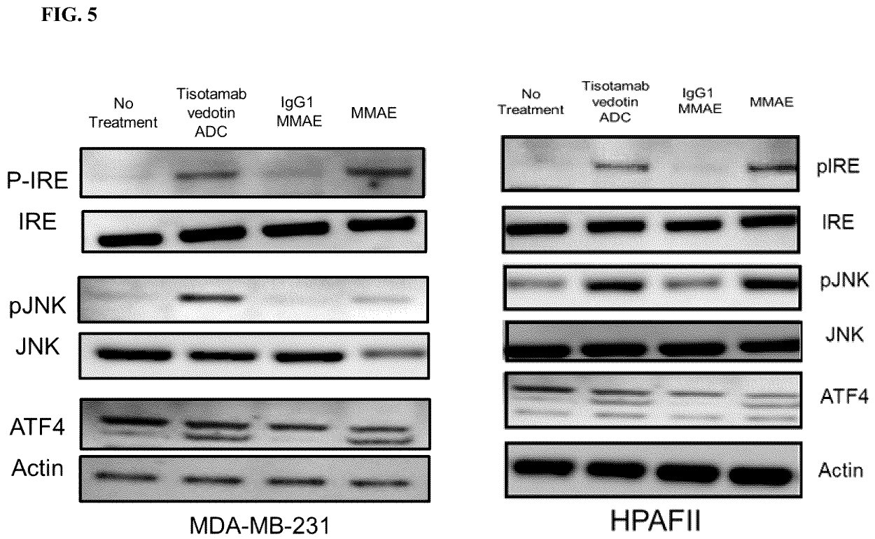

Auristatin ADC payloads disrupt the microtubule networks resulting in altered ER localization and function, which ultimately results in ER stress. Cells exposed to Tissue Factor directed antibody linked to the monomethyl auristatin E payload (MMAE), i.e., tisotumab vedotin (an antibody drug conjugate or ADC), undergo cell death and as they do release the ICD related molecules ATP (FIG. 4A) and HMGB1 (FIG. 4C). The release of these molecules is specific to tisotumab vedotin ADC and MMAE treatment and occurs across multiple Tissue Factor positive cell lines (FIG. 4B).

### Example 6: Auristatins, Both Free and ADC-Loaded, are Able to Induce ER Stress Pathways that are Critical for Immunogenic Cell Death

Induction of cell death and release of ICD danger signals occurs concomitant with initiation of an ER stress response. Two Tissue Factor positive cell lines, HPAFII (pancreatic carcinoma) and MDA-MB-231 (breast cell carcinoma) were exposed to tisotumab vedotin ADC, an Isotype-MMAE ADC (H00-MMAE, IgG1 MMAE), or free MMAE for 18 hours and induction of ER stress monitored by western blot analysis. Phosphorylation of inositol-requiring transmembrane kinase/endonuclease 1 (IRE1) was detected after treatment with tisotumab vedotin ADC or MMAE free drug (FIG. 5). Activation of the IRE1 downstream effector Jun N-terminal kinase (JNK) also occurred as monitored by increased phosphorylation. Furthermore, activation of the PKR-like ER kinase (PERK) secondary ER stress pathway was detected via upregulation of ATF4 cleavage. These data indicate that auristatins, both free and ADC-loaded, are able to induce ER stress pathways that are critical for ICD and the expression of tumor antigens on apoptotic cell surfaces. The ability for auristatins to prime the immune system to recognize tumor antigens opens the door for a myriad of combinatorial therapeutic options.

### Example 7: Tisotumab Vedotin ADC and MMAE Killed Tissue Factor Positive Cells Elicit Strong Chemotactic and Inflammatory Mediators from Monocyte/Macrophages after Uptake of Dead Cells

Investigations into the mechanisms of action of therapeutics for oncology extend long past cytolysis of tumor cells. The growing focus on immunotherapy highlights the processes involved in clearing dying tumor cells, as well as engaging the patient's immune system to provoke antitumor responses. The method of cell death and subsequent clearance of cell debris speaks volumes to the level of engagement and stimulation of the immune system to generate targeted responses against the tumor cells

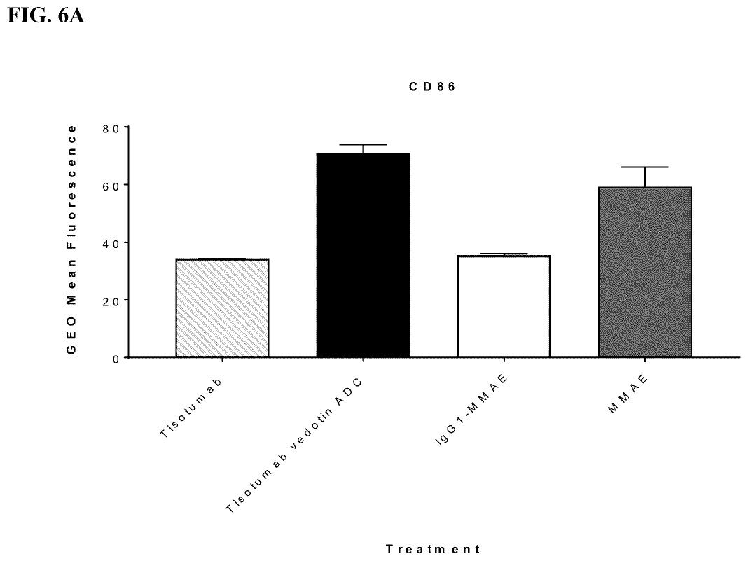

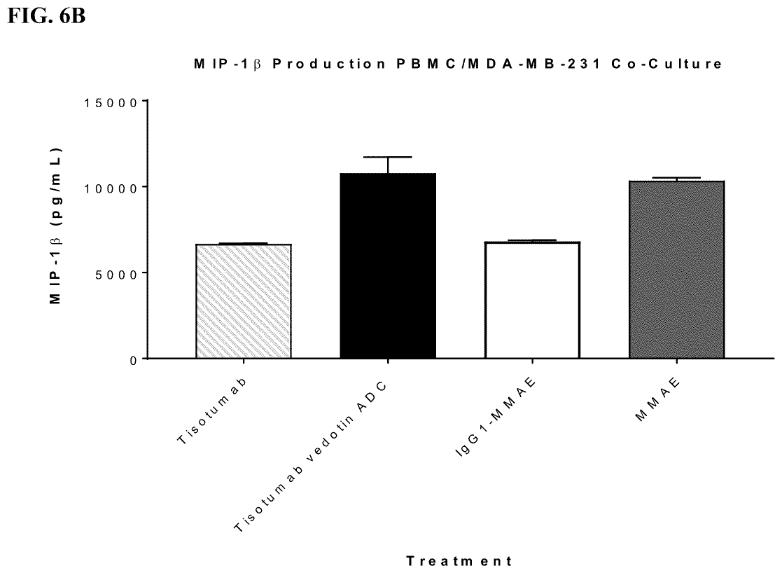

Immunogenic cell death, as mediated by MMAE, is regulated cell death that activates adaptive immune responses against antigens from dead and dying tumor cells, and allows for the generation of robust innate immune cell activation and subsequent cytotoxic T cell responses targeted towards specific tumor cell antigens. Here, we demonstrated that tisotumab vedotin ADC and MMAE killed Tissue Factor positive cells elicit strong chemotactic and inflammatory mediators from monocyte/macrophages after uptake of dead cells (FIGS. 6A and 6B). Furthermore, these monocyte/macrophages conditioned by ICD-killed cells promote activation of T cells, as evidenced by production of signature inflammatory cytokines associated with cytotoxic T cell responses.

### Example 8: Tisotumab Vedotin Induces ICD which Results in Innate Immune Cells Activation and Secondary T Cell Responses that can be Amplified with PD1 Targeted Agents

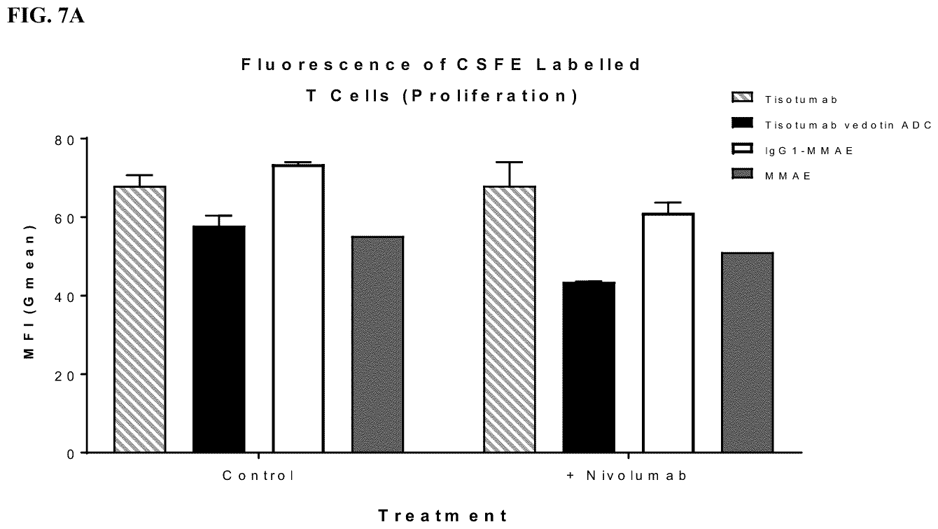

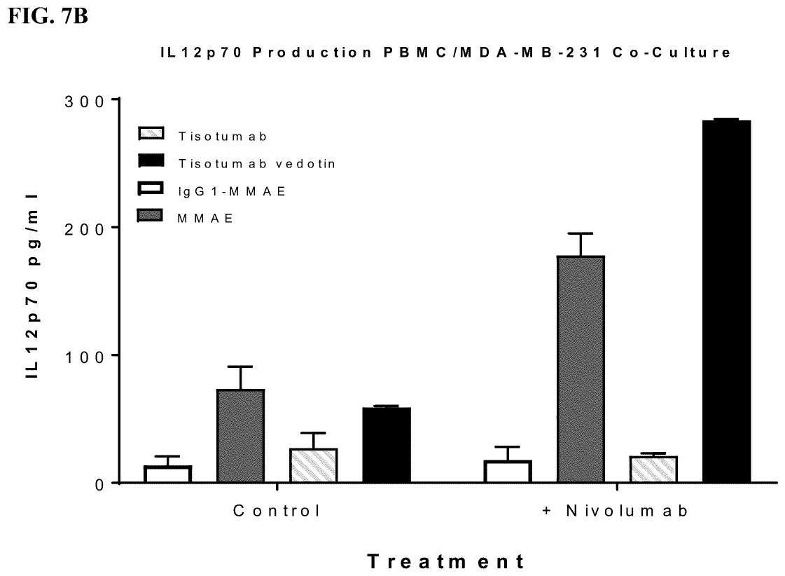

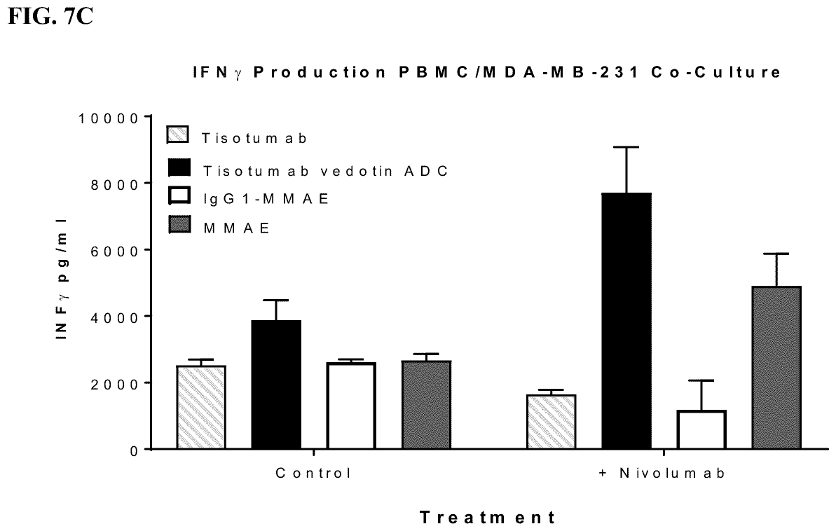

Induction of the innate immune response following exposure to cancer cells undergoing ICD sets up secondary T cell activation, which can be enhanced by concomitant anti-PD1 treatment. Tissue Factor positive MDA-MB-231 cells exposed to tisotumab vedotin or MMAE when fed to CSFE labeled human PBMCs for 48 hours drove T cell proliferation as monitored by CSFE dilution (FIG. 7A) and production of T cell specific cytokines such as IL12_{P}70 and IFNγ (FIGS. 7B and 7C). Tissue Factor targeting antibody alone or an isotype-MMAE ADC (Isotype-MMAE, IgG1-MMAE) did not elicit these responses. These data support that tisotumab vedotin induces ICD which results in innate immune cells activation and secondary T cell responses that can be amplified with PD1 targeted agents.

1. A method of treating cancer in a subject, the method comprising administering to the subject an anti-PD-1 antibody or an antigen-binding fragment thereof, wherein the antibody binds to Programmed Death-1 (PD-1) and inhibits PD-1 activity, wherein the anti-PD-1 antibody or antigen-binding fragment thereof comprises the complementary determining regions (CDRs) of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof, and an antibody-drug conjugate that binds to tissue factor (TF), wherein the antibody-drug conjugate comprises an anti-TF antibody or an antigen-binding fragment thereof conjugated to an auristatin or a functional analog thereof or a functional derivative thereof.

2. (canceled)

3. The method of claim 1, wherein the anti-PD-1 antibody or antigen-binding fragment thereof comprises the heavy chain variable region and the light chain variable region of an antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof.

4. (canceled)

5. The method of claim 1, wherein the anti-PD-1 antibody or antigen-binding fragment thereof is selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof.

6. (canceled)

7. The method of claim 1, wherein the antibody-drug conjugate is administered at a dose ranging from about 0.9 mg/kg to about 2.1 mg/kg.

8. The method of claim 7, wherein the antibody-drug conjugate is administered at a dose of about 2.0 mg/kg.

9. (canceled)

10. The method of claim 1, wherein the antibody-drug conjugate is administered once about every 1 week, once about every 2 weeks, once about every 3 weeks or once about every 4 weeks.

11-15. (canceled)

16. The method of claim 1, wherein the anti-PD-1 antibody or antigen-binding fragment thereof is administered at a dose ranging from about 0.5 mg/kg to about 4.1 mg/kg.

17. The method of claim 1, wherein the anti-PD-1 antibody or antigen-binding fragment thereof is administered at a flat dose ranging from about 50 mg to about 500 mg.

18-19. (canceled)

20. The method of claim 1, wherein the anti-PD-1 antibody or antigen-binding fragment thereof is administered once about every 1 week, once about every 2 weeks, once about every 3 weeks or once about every 4 weeks.

21. (canceled)

22. The method of claim 1, wherein the cancer is breast cancer.

23. The method of claim 1, wherein the cancer is cervical cancer.

24-26. (canceled)

27. The method of claim 23, wherein the cervical cancer is an adenocarcinoma, an adenosquamous carcinoma or a squamous cell carcinoma.

28-31. (canceled)

32. The method of claim 1, wherein the auristatin is monomethyl auristatin E (MMAE).

33. (canceled)

34. The method of claim 1, wherein the anti-TF antibody or antigen-binding fragment thereof of the antibody-drug conjugate comprises a heavy chain variable region and a light chain variable region, wherein the heavy chain variable region comprises:
(i) a CDR-H1 comprising the amino acid sequence of SEQ ID NO:1;
(ii) a CDR-H2 comprising the amino acid sequence of SEQ ID NO:2; and
(iii) a CDR-H3 comprising the amino acid sequence of SEQ ID NO:3; and wherein the light chain variable region comprises:
(i) a CDR-L1 comprising the amino acid sequence of SEQ ID NO:4;
(ii) a CDR-L2 comprising the amino acid sequence of SEQ ID NO:5; and
(iii) a CDR-L3 comprising the amino acid sequence of SEQ ID NO:6.

35. (canceled)

36. The method of claim 1, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.

37. The method of claim 1, wherein the anti-TF antibody of the antibody-drug conjugate is tisotumab.

38. The method of claim 1, wherein the antibody-drug conjugate further comprises a linker between the anti-TF antibody or antigen-binding fragment thereof and the auristatin.

39-41. (canceled)

42. The method of claim 38, wherein the linker is attached to MMAE (vcMMAE), wherein the antibody-drug conjugate has the following structure: 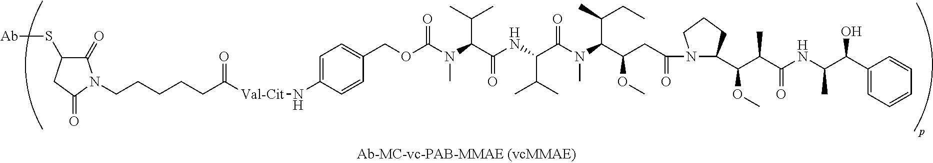
wherein p denotes a number from 1 to 8, S represents a sulphydryl residue of the anti-TF antibody, and Ab designates the anti-TF antibody or antigen-binding fragment thereof.

43. (canceled)

44. The method of claim 1, wherein the antibody-drug conjugate is tisotumab vedotin.

45-48. (canceled)

49. The method of claim 1, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express TF.

50. The method of claim 1, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express PD-L1.

51. (canceled)

52. The method of claim 1, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express PD-1.

53. The method of claim 1, wherein one or more therapeutic effects in the subject is improved after administration of the antibody-drug conjugate and the anti-PD-1 antibody or antigen-binding fragment thereof relative to a baseline.

54. The method of claim 53, wherein the one or more therapeutic effects is selected from the group consisting of: size of a tumor derived from the cancer, objective response rate, duration of response, time to response, progression free survival, and overall survival.

55-59. (canceled)

60. The method of claim 1, wherein the subject has one or more adverse events and is further administered an additional therapeutic agent to eliminate or reduce the severity of the one or more adverse events.

61. (canceled)

62. The method of claim 60, wherein the one or more adverse events is anemia, abdominal pain, hemorrhage, hyperthyroidism, hypothyroidism, hypokalemia, hyponatremia, epistaxis, fatigue, nausea, alopecia, conjunctivitis, keratitis, conjunctival ulceration, constipation, decreased appetite, diarrhea, vomiting, peripheral neuropathy, or general physical health deterioration.

63-64. (canceled)

65. The method of claim 60, wherein the one or more adverse events is conjunctivitis, conjunctival ulceration, and/or keratitis and the additional agent is a preservative-free lubricating eye drop, an ocular vasoconstrictor, antibiotic, and/or a steroid eye drop.

66-68. (canceled)

69. A kit comprising:
(a) an antibody or an antigen-binding fragment thereof, wherein the antibody binds to Programmed Death-1 (PD-1) and inhibits PD-1 activity;
(b) a dosage ranging from about 0.9 mg/kg to about 2.1 mg/kg of an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof; and
(c) instructions for use of the anti-PD-1 antibody or antigen-binding fragment thereof and the antibody drug conjugate according to the method of claim 1.
